SEQUENCE LISTING (193 sequences)

SEQ ID NO: 1 - DNA, 99 bp, synthetic construct
  source 1..99: mol_type = other DNA
ccctccgatacacaaagacaccgacaactttctttgcgtacagcaatcaggcgcgcccgatcggaattcttaattaatggtggactctatgaccgttca

SEQ ID NO: 2 - DNA, 99 bp, synthetic construct
  source 1..99: mol_type = other DNA
ccctccgataacagacgactacaaacggaatcgatgcgtacagcaatcaggcgcgcccgatcggaattcttaattaatggtggactctatgaccgttca

SEQ ID NO: 3 - DNA, 99 bp, synthetic construct
  source 1..99: mol_type = other DNA
ccctccgataaggtctacctcgctaacaccactgtgcgtacagcaatcaggcgcgcccgatcggaattcttaattaatggtggactctatgaccgttca

SEQ ID NO: 4 - DNA, 99 bp, synthetic construct
  source 1..99: mol_type = other DNA
ccctccgatagttcctcgtgcagtgtcaagagattgcgtacagcaatcaggcgcgcccgatcggaattcttaattaatggtggactctatgaccgttca

SEQ ID NO: 5 - DNA, 99 bp, synthetic construct
  source 1..99: mol_type = other DNA
ccctccgatattgcgtcctgttacgagaactcattgcgtacagcaatcaggcgcgcccgatcggaattcttaattaatggtggactctatgaccgttca

SEQ ID NO: 6 - DNA, 99 bp, synthetic construct
  source 1..99: mol_type = other DNA
ccctccgataccgatccttgtggcttctaacttctgcgtacagcaatcaggcgcgcccgatcggaattcttaattaatggtggactctatgaccgttca

SEQ ID NO: 7 - DNA, 99 bp, synthetic construct
  source 1..99: mol_type = other DNA
ccctccgatagtttgtcatactcgtgtgctcacctgcgtacagcaatcaggcgcgcccgatcggaattcttaattaatggtggactctatgaccgttca

SEQ ID NO: 8 - DNA, 99 bp, synthetic construct
  source 1..99: mol_type = other DNA
ccctccgatatacagtccgagcctcatgtgatcttgcgtacagcaatcaggcgcgcccgatcggaattcttaattaatggtggactctatgaccgttca

SEQ ID NO: 9 - DNA, 99 bp, synthetic construct
  source 1..99: mol_type = other DNA
ccctccgataaccgagatcctacgaatggagtgttgcgtacagcaatcaggcgcgcccgatcggaattcttaattaatggtggactctatgaccgttca

SEQ ID NO: 10 - DNA, 99 bp, synthetic construct
  source 1..99: mol_type = other DNA
ccctccgatatagctgactgtcttccataccgactgcgtacagcaatcaggcgcgcccgatcggaattcttaattaatggtggactctatgaccgttca

SEQ ID NO: 11 - DNA, 99 bp, synthetic construct
  source 1..99: mol_type = other DNA
ccctccgatatagtttggatgaccaaggatagccgaattcgatcgttaattaatagggataacagggtaatggcgcgccatggtggactctatgaccat

SEQ ID NO: 12 - DNA, 100 bp, synthetic construct
  source 1..100: mol_type = other DNA
catggcgcgccattaccctgttatccctattaattaacgatcgaattcggctatccttggtcatccaaactatatcggagggccctaaccctaaccctaa

SEQ ID NO: 13 - DNA, 100 bp, synthetic construct
  source 1..100: mol_type = other DNA
catggcgcgccattaccctgttatccctattaattaacgatcgaattcggctatccttggtcatccaaactatatcggagggcctaaccctaaccctaac

SEQ ID NO: 14 - DNA, 100 bp, synthetic construct
  source 1..100: mol_type = other DNA
catggcgcgccattaccctgttatccctattaattaacgatcgaattcggctatccttggtcatccaaactatatcggagggctaaccctaaccctaacc

SEQ ID NO: 15 - DNA, 100 bp, synthetic construct
  source 1..100: mol_type = other DNA
catggcgcgccattaccctgttatccctattaattaacgatcgaattcggctatccttggtcatccaaactatatcggagggtaaccctaaccctaaccc

SEQ ID NO: 16 - DNA, 100 bp, synthetic construct
  source 1..100: mol_type = other DNA
catggcgcgccattaccctgttatccctattaattaacgatcgaattcggctatccttggtcatccaaactatatcggagggaaccctaaccctaaccct

SEQ ID NO: 17 - DNA, 100 bp, synthetic construct
  source 1..100: mol_type = other DNA
catggcgcgccattaccctgttatccctattaattaacgatcgaattcggctatccttggtcatccaaactatatcggagggaccctaaccctaacccta

SEQ ID NO: 18 - DNA, 99 bp, synthetic construct
  source 1..99: mol_type = other DNA
ccctccgatactttcgttgttgactcgacggtaggaattcgatcgttaattaatagggataacagggtaatggcgcgccatggtggactctatgaccat

SEQ ID NO: 19 - DNA, 100 bp, synthetic construct
  source 1..100: mol_type = other DNA
catggcgcgccattaccctgttatccctattaattaacgatcgaattcctaccgtcgagtcaacaacgaaagtatcggagggccctaaccctaaccctaa

SEQ ID NO: 20 - DNA, 100 bp, synthetic construct
  source 1..100: mol_type = other DNA
catggcgcgccattaccctgttatccctattaattaacgatcgaattcctaccgtcgagtcaacaacgaaagtatcggagggcctaaccctaaccctaac

SEQ ID NO: 21 - DNA, 100 bp, synthetic construct
  source 1..100: mol_type = other DNA
catggcgcgccattaccctgttatccctattaattaacgatcgaattcctaccgtcgagtcaacaacgaaagtatcggagggctaaccctaaccctaacc

SEQ ID NO: 22 - DNA, 100 bp, synthetic construct
  source 1..100: mol_type = other DNA
catggcgcgccattaccctgttatccctattaattaacgatcgaattcctaccgtcgagtcaacaacgaaagtatcggagggtaaccctaaccctaaccc

SEQ ID NO: 23 - DNA, 100 bp, synthetic construct
  source 1..100: mol_type = other DNA
catggcgcgccattaccctgttatccctattaattaacgatcgaattcctaccgtcgagtcaacaacgaaagtatcggagggaaccctaaccctaaccct

SEQ ID NO: 24 - DNA, 100 bp, synthetic construct
  source 1..100: mol_type = other DNA
catggcgcgccattaccctgttatccctattaattaacgatcgaattcctaccgtcgagtcaacaacgaaagtatcggagggaccctaaccctaacccta

SEQ ID NO: 25 - DNA, 99 bp, synthetic construct
  source 1..99: mol_type = other DNA
ccctccgataatggactttggtaacttcctgcgttgcgtacagcaatcaggcgcgccgaattccgatcgttaattaatggtggactctatgaccgttca

SEQ ID NO: 26 - DNA, 100 bp, synthetic construct
  source 1..100: mol_type = other DNA
caccattaattaacgatcggaattcggcgcgcctgattgctgtacgcaacgcaggaagttaccaaagtccattatcggagggccctaaccctaaccctaa

SEQ ID NO: 27 - DNA, 100 bp, synthetic construct
  source 1..100: mol_type = other DNA
caccattaattaacgatcggaattcggcgcgcctgattgctgtacgcaacgcaggaagttaccaaagtccattatcggagggcctaaccctaaccctaac

SEQ ID NO: 28 - DNA, 100 bp, synthetic construct
  source 1..100: mol_type = other DNA
caccattaattaacgatcggaattcggcgcgcctgattgctgtacgcaacgcaggaagttaccaaagtccattatcggagggctaaccctaaccctaacc

SEQ ID NO: 29 - DNA, 100 bp, synthetic construct
  source 1..100: mol_type = other DNA
caccattaattaacgatcggaattcggcgcgcctgattgctgtacgcaacgcaggaagttaccaaagtccattatcggagggtaaccctaaccctaaccc

SEQ ID NO: 30 - DNA, 100 bp, synthetic construct
  source 1..100: mol_type = other DNA
caccattaattaacgatcggaattcggcgcgcctgattgctgtacgcaacgcaggaagttaccaaagtccattatcggagggaaccctaaccctaaccct

SEQ ID NO: 31 - DNA, 100 bp, synthetic construct
  source 1..100: mol_type = other DNA
caccattaattaacgatcggaattcggcgcgcctgattgctgtacgcaacgcaggaagttaccaaagtccattatcggagggaccctaaccctaacccta

SEQ ID NO: 32 - DNA, 100 bp, synthetic construct
  source 1..100: mol_type = other DNA
agagtccaccatgaattccactgacaatgtaactgattgctgtacgcacaggtagaaagaagcagaatcggatatcggagggccctaaccctaaccctaa

SEQ ID NO: 33 - DNA, 100 bp, synthetic construct
  source 1..100: mol_type = other DNA
agagtccaccatgaattccactgacaatgtaactgattgctgtacgcacaggtagaaagaagcagaatcggatatcggagggcctaaccctaaccctaac

SEQ ID NO: 34 - DNA, 100 bp, synthetic construct
  source 1..100: mol_type = other DNA
agagtccaccatgaattccactgacaatgtaactgattgctgtacgcacaggtagaaagaagcagaatcggatatcggagggctaaccctaaccctaacc

SEQ ID NO: 35 - DNA, 100 bp, synthetic construct
  source 1..100: mol_type = other DNA
agagtccaccatgaattccactgacaatgtaactgattgctgtacgcacaggtagaaagaagcagaatcggatatcggagggtaaccctaaccctaaccc

SEQ ID NO: 36 - DNA, 100 bp, synthetic construct
  source 1..100: mol_type = other DNA
agagtccaccatgaattccactgacaatgtaactgattgctgtacgcacaggtagaaagaagcagaatcggatatcggagggaaccctaaccctaaccct

SEQ ID NO: 37 - DNA, 100 bp, synthetic construct
  source 1..100: mol_type = other DNA
agagtccaccatgaattccactgacaatgtaactgattgctgtacgcacaggtagaaagaagcagaatcggatatcggagggaccctaaccctaacccta

SEQ ID NO: 38 - DNA, 99 bp, synthetic construct
  source 1..99: mol_type = other DNA
ccctccgatattctcagtcttcctccagacaaggtgcgtacagcaatcaggcgcgccgaattccgatcgttaattaatggtggactctatgaccgttca

SEQ ID NO: 39 - DNA, 100 bp, synthetic construct
  source 1..100: mol_type = other DNA
caccattaattaacgatcggaattcggcgcgcctgattgctgtacgcaccttgtctggaggaagactgagaatatcggagggccctaaccctaaccctaa

SEQ ID NO: 40 - DNA, 100 bp, synthetic construct
  source 1..100: mol_type = other DNA
caccattaattaacgatcggaattcggcgcgcctgattgctgtacgcaccttgtctggaggaagactgagaatatcggagggcctaaccctaaccctaac

SEQ ID NO: 41 - DNA, 100 bp, synthetic construct
  source 1..100: mol_type = other DNA
caccattaattaacgatcggaattcggcgcgcctgattgctgtacgcaccttgtctggaggaagactgagaatatcggagggctaaccctaaccctaacc

SEQ ID NO: 42 - DNA, 100 bp, synthetic construct
  source 1..100: mol_type = other DNA
caccattaattaacgatcggaattcggcgcgcctgattgctgtacgcaccttgtctggaggaagactgagaatatcggagggtaaccctaaccctaaccc

SEQ ID NO: 43 - DNA, 100 bp, synthetic construct
  source 1..100: mol_type = other DNA
caccattaattaacgatcggaattcggcgcgcctgattgctgtacgcaccttgtctggaggaagactgagaatatcggagggaaccctaaccctaaccct

SEQ ID NO: 44 - DNA, 100 bp, synthetic construct
  source 1..100: mol_type = other DNA
caccattaattaacgatcggaattcggcgcgcctgattgctgtacgcaccttgtctggaggaagactgagaatatcggagggaccctaaccctaacccta

SEQ ID NO: 45 - DNA, 99 bp, synthetic construct
  source 1..99: mol_type = other DNA
ccctccgatagaatctaagcaaacacgaaggtggtgcgtacagcaatcaggcgcgccgaattccgatcgttaattaatggtggactctatgaccgttca

SEQ ID NO: 46 - DNA, 100 bp, synthetic construct
  source 1..100: mol_type = other DNA
caccattaattaacgatcggaattcggcgcgcctgattgctgtacgcaccaccttcgtgtttgcttagattctatcggagggccctaaccctaaccctaa

SEQ ID NO: 47 - DNA, 100 bp, synthetic construct
  source 1..100: mol_type = other DNA
caccattaattaacgatcggaattcggcgcgcctgattgctgtacgcaccaccttcgtgtttgcttagattctatcggagggcctaaccctaaccctaac

SEQ ID NO: 48 - DNA, 100 bp, synthetic construct
  source 1..100: mol_type = other DNA
caccattaattaacgatcggaattcggcgcgcctgattgctgtacgcaccaccttcgtgtttgcttagattctatcggagggctaaccctaaccctaacc

SEQ ID NO: 49 - DNA, 100 bp, synthetic construct
  source 1..100: mol_type = other DNA
caccattaattaacgatcggaattcggcgcgcctgattgctgtacgcaccaccttcgtgtttgcttagattctatcggagggtaaccctaaccctaaccc

SEQ ID NO: 50 - DNA, 100 bp, synthetic construct
  source 1..100: mol_type = other DNA
caccattaattaacgatcggaattcggcgcgcctgattgctgtacgcaccaccttcgtgtttgcttagattctatcggagggaaccctaaccctaaccct

SEQ ID NO: 51 - DNA, 100 bp, synthetic construct
  source 1..100: mol_type = other DNA
caccattaattaacgatcggaattcggcgcgcctgattgctgtacgcaccaccttcgtgtttgcttagattctatcggagggaccctaaccctaacccta

SEQ ID NO: 52 - DNA, 99 bp, synthetic construct
  source 1..99: mol_type = other DNA
ccctccgatatcttctactaccgatccgaagcagtgcgtacagcaatcaggcgcgccgaattccgatcgttaattaatggtggactctatgaccgttca

SEQ ID NO: 53 - DNA, 100 bp, synthetic construct
  source 1..100: mol_type = other DNA
caccattaattaacgatcggaattcggcgcgcctgattgctgtacgcactgcttcggatcggtagtagaagatatcggagggccctaaccctaaccctaa

SEQ ID NO: 54 - DNA, 100 bp, synthetic construct
  source 1..100: mol_type = other DNA
caccattaattaacgatcggaattcggcgcgcctgattgctgtacgcactgcttcggatcggtagtagaagatatcggagggcctaaccctaaccctaac

SEQ ID NO: 55 - DNA, 100 bp, synthetic construct
  source 1..100: mol_type = other DNA
caccattaattaacgatcggaattcggcgcgcctgattgctgtacgcactgcttcggatcggtagtagaagatatcggagggctaaccctaaccctaacc

SEQ ID NO: 56 - DNA, 100 bp, synthetic construct
  source 1..100: mol_type = other DNA
caccattaattaacgatcggaattcggcgcgcctgattgctgtacgcactgcttcggatcggtagtagaagatatcggagggtaaccctaaccctaaccc

SEQ ID NO: 57 - DNA, 100 bp, synthetic construct
  source 1..100: mol_type = other DNA
caccattaattaacgatcggaattcggcgcgcctgattgctgtacgcactgcttcggatcggtagtagaagatatcggagggaaccctaaccctaaccct

SEQ ID NO: 58 - DNA, 100 bp, synthetic construct
  source 1..100: mol_type = other DNA
caccattaattaacgatcggaattcggcgcgcctgattgctgtacgcactgcttcggatcggtagtagaagatatcggagggaccctaaccctaacccta

SEQ ID NO: 59 - DNA, 99 bp, synthetic construct
  source 1..99: mol_type = other DNA
ccctccgataacggtatgtcgagttccaggactagaattctagggataacagggtaatgatcgttaattaaggcgcgccatggtggactctatgaccat

SEQ ID NO: 60 - DNA, 100 bp, synthetic construct
  source 1..100: mol_type = other DNA
catggcgcgccttaattaacgatcattaccctgttatccctagaattctagtcctggaactcgacataccgttatcggagggccctaaccctaaccctaa

SEQ ID NO: 61 - DNA, 100 bp, synthetic construct
  source 1..100: mol_type = other DNA
catggcgcgccttaattaacgatcattaccctgttatccctagaattctagtcctggaactcgacataccgttatcggagggcctaaccctaaccctaac

SEQ ID NO: 62 - DNA, 100 bp, synthetic construct
  source 1..100: mol_type = other DNA
catggcgcgccttaattaacgatcattaccctgttatccctagaattctagtcctggaactcgacataccgttatcggagggctaaccctaaccctaacc

SEQ ID NO: 63 - DNA, 100 bp, synthetic construct
  source 1..100: mol_type = other DNA
catggcgcgccttaattaacgatcattaccctgttatccctagaattctagtcctggaactcgacataccgttatcggagggtaaccctaaccctaaccc

SEQ ID NO: 64 - DNA, 100 bp, synthetic construct
  source 1..100: mol_type = other DNA
catggcgcgccttaattaacgatcattaccctgttatccctagaattctagtcctggaactcgacataccgttatcggagggaaccctaaccctaaccct

SEQ ID NO: 65 - DNA, 100 bp, synthetic construct
  source 1..100: mol_type = other DNA
catggcgcgccttaattaacgatcattaccctgttatccctagaattctagtcctggaactcgacataccgttatcggagggaccctaaccctaacccta

SEQ ID NO: 66 - DNA, 100 bp, synthetic construct
  source 1..100: mol_type = other DNA
caccattaattaagaattccgatcgggcgcgcctgattgctgtacgcaaagaaagttgtcggtgtctttgtgtatcggagggccctaaccctaaccctaa

SEQ ID NO: 67 - DNA, 100 bp, synthetic construct
  source 1..100: mol_type = other DNA
caccattaattaagaattccgatcgggcgcgcctgattgctgtacgcaaagaaagttgtcggtgtctttgtgtatcggagggcctaaccctaaccctaac

SEQ ID NO: 68 - DNA, 100 bp, synthetic construct
  source 1..100: mol_type = other DNA
caccattaattaagaattccgatcgggcgcgcctgattgctgtacgcaaagaaagttgtcggtgtctttgtgtatcggagggctaaccctaaccctaacc

SEQ ID NO: 69 - DNA, 100 bp, synthetic construct
  source 1..100: mol_type = other DNA
caccattaattaagaattccgatcgggcgcgcctgattgctgtacgcaaagaaagttgtcggtgtctttgtgtatcggagggtaaccctaaccctaaccc

SEQ ID NO: 70 - DNA, 100 bp, synthetic construct
  source 1..100: mol_type = other DNA
caccattaattaagaattccgatcgggcgcgcctgattgctgtacgcaaagaaagttgtcggtgtctttgtgtatcggagggaaccctaaccctaaccct

SEQ ID NO: 71 - DNA, 100 bp, synthetic construct
  source 1..100: mol_type = other DNA
caccattaattaagaattccgatcgggcgcgcctgattgctgtacgcaaagaaagttgtcggtgtctttgtgtatcggagggaccctaaccctaacccta

SEQ ID NO: 72 - DNA, 100 bp, synthetic construct
  source 1..100: mol_type = other DNA
caccattaattaagaattccgatcgggcgcgcctgattgctgtacgcatcgattccgtttgtagtcgtctgttatcggagggccctaaccctaaccctaa

SEQ ID NO: 73 - DNA, 100 bp, synthetic construct
  source 1..100: mol_type = other DNA
caccattaattaagaattccgatcgggcgcgcctgattgctgtacgcatcgattccgtttgtagtcgtctgttatcggagggcctaaccctaaccctaac

SEQ ID NO: 74 - DNA, 100 bp, synthetic construct
  source 1..100: mol_type = other DNA
caccattaattaagaattccgatcgggcgcgcctgattgctgtacgcatcgattccgtttgtagtcgtctgttatcggagggctaaccctaaccctaacc

SEQ ID NO: 75 - DNA, 100 bp, synthetic construct
  source 1..100: mol_type = other DNA
caccattaattaagaattccgatcgggcgcgcctgattgctgtacgcatcgattccgtttgtagtcgtctgttatcggagggtaaccctaaccctaaccc

SEQ ID NO: 76 - DNA, 100 bp, synthetic construct
  source 1..100: mol_type = other DNA
caccattaattaagaattccgatcgggcgcgcctgattgctgtacgcatcgattccgtttgtagtcgtctgttatcggagggaaccctaaccctaaccct

SEQ ID NO: 77 - DNA, 100 bp, synthetic construct
  source 1..100: mol_type = other DNA
caccattaattaagaattccgatcgggcgcgcctgattgctgtacgcatcgattccgtttgtagtcgtctgttatcggagggaccctaaccctaacccta

SEQ ID NO: 78 - DNA, 100 bp, synthetic construct
  source 1..100: mol_type = other DNA
caccattaattaagaattccgatcgggcgcgcctgattgctgtacgcacagtggtgttagcgaggtagaccttatcggagggccctaaccctaaccctaa

SEQ ID NO: 79 - DNA, 100 bp, synthetic construct
  source 1..100: mol_type = other DNA
caccattaattaagaattccgatcgggcgcgcctgattgctgtacgcacagtggtgttagcgaggtagaccttatcggagggcctaaccctaaccctaac

SEQ ID NO: 80 - DNA, 100 bp, synthetic construct
  source 1..100: mol_type = other DNA
caccattaattaagaattccgatcgggcgcgcctgattgctgtacgcacagtggtgttagcgaggtagaccttatcggagggctaaccctaaccctaacc

SEQ ID NO: 81 - DNA, 100 bp, synthetic construct
  source 1..100: mol_type = other DNA
caccattaattaagaattccgatcgggcgcgcctgattgctgtacgcacagtggtgttagcgaggtagaccttatcggagggtaaccctaaccctaaccc

SEQ ID NO: 82 - DNA, 100 bp, synthetic construct
  source 1..100: mol_type = other DNA
caccattaattaagaattccgatcgggcgcgcctgattgctgtacgcacagtggtgttagcgaggtagaccttatcggagggaaccctaaccctaaccct

SEQ ID NO: 83 - DNA, 100 bp, synthetic construct
  source 1..100: mol_type = other DNA
caccattaattaagaattccgatcgggcgcgcctgattgctgtacgcacagtggtgttagcgaggtagaccttatcggagggaccctaaccctaacccta

SEQ ID NO: 84 - DNA, 100 bp, synthetic construct
  source 1..100: mol_type = other DNA
caccattaattaagaattccgatcgggcgcgcctgattgctgtacgcaatctcttgacactgcacgaggaactatcggagggccctaaccctaaccctaa

SEQ ID NO: 85 - DNA, 100 bp, synthetic construct
  source 1..100: mol_type = other DNA
caccattaattaagaattccgatcgggcgcgcctgattgctgtacgcaatctcttgacactgcacgaggaactatcggagggcctaaccctaaccctaac

SEQ ID NO: 86 - DNA, 100 bp, synthetic construct
  source 1..100: mol_type = other DNA
caccattaattaagaattccgatcgggcgcgcctgattgctgtacgcaatctcttgacactgcacgaggaactatcggagggctaaccctaaccctaacc

SEQ ID NO: 87 - DNA, 100 bp, synthetic construct
  source 1..100: mol_type = other DNA
caccattaattaagaattccgatcgggcgcgcctgattgctgtacgcaatctcttgacactgcacgaggaactatcggagggtaaccctaaccctaaccc

SEQ ID NO: 88 - DNA, 100 bp, synthetic construct
  source 1..100: mol_type = other DNA
caccattaattaagaattccgatcgggcgcgcctgattgctgtacgcaatctcttgacactgcacgaggaactatcggagggaaccctaaccctaaccct

SEQ ID NO: 89 - DNA, 100 bp, synthetic construct
  source 1..100: mol_type = other DNA
caccattaattaagaattccgatcgggcgcgcctgattgctgtacgcaatctcttgacactgcacgaggaactatcggagggaccctaaccctaacccta

SEQ ID NO: 90 - DNA, 100 bp, synthetic construct
  source 1..100: mol_type = other DNA
caccattaattaagaattccgatcgggcgcgcctgattgctgtacgcaatgagttctcgtaacaggacgcaatatcggagggccctaaccctaaccctaa

SEQ ID NO: 91 - DNA, 100 bp, synthetic construct
  source 1..100: mol_type = other DNA
caccattaattaagaattccgatcgggcgcgcctgattgctgtacgcaatgagttctcgtaacaggacgcaatatcggagggcctaaccctaaccctaac

SEQ ID NO: 92 - DNA, 100 bp, synthetic construct
  source 1..100: mol_type = other DNA
caccattaattaagaattccgatcgggcgcgcctgattgctgtacgcaatgagttctcgtaacaggacgcaatatcggagggctaaccctaaccctaacc

SEQ ID NO: 93 - DNA, 100 bp, synthetic construct
  source 1..100: mol_type = other DNA
caccattaattaagaattccgatcgggcgcgcctgattgctgtacgcaatgagttctcgtaacaggacgcaatatcggagggtaaccctaaccctaaccc

SEQ ID NO: 94 - DNA, 100 bp, synthetic construct
  source 1..100: mol_type = other DNA
caccattaattaagaattccgatcgggcgcgcctgattgctgtacgcaatgagttctcgtaacaggacgcaatatcggagggaaccctaaccctaaccct

SEQ ID NO: 95 - DNA, 100 bp, synthetic construct
  source 1..100: mol_type = other DNA
caccattaattaagaattccgatcgggcgcgcctgattgctgtacgcaatgagttctcgtaacaggacgcaatatcggagggaccctaaccctaacccta

SEQ ID NO: 96 - DNA, 100 bp, synthetic construct
  source 1..100: mol_type = other DNA
caccattaattaagaattccgatcgggcgcgcctgattgctgtacgcagaagttagaagccacaaggatcggtatcggagggccctaaccctaaccctaa

SEQ ID NO: 97 - DNA, 100 bp, synthetic construct
  source 1..100: mol_type = other DNA
caccattaattaagaattccgatcgggcgcgcctgattgctgtacgcagaagttagaagccacaaggatcggtatcggagggcctaaccctaaccctaac

SEQ ID NO: 98 - DNA, 100 bp, synthetic construct
  source 1..100: mol_type = other DNA
caccattaattaagaattccgatcgggcgcgcctgattgctgtacgcagaagttagaagccacaaggatcggtatcggagggctaaccctaaccctaacc

SEQ ID NO: 99 - DNA, 100 bp, synthetic construct
  source 1..100: mol_type = other DNA
caccattaattaagaattccgatcgggcgcgcctgattgctgtacgcagaagttagaagccacaaggatcggtatcggagggtaaccctaaccctaaccc

SEQ ID NO: 100 - DNA, 100 bp, synthetic construct
  source 1..100: mol_type = other DNA
caccattaattaagaattccgatcgggcgcgcctgattgctgtacgcagaagttagaagccacaaggatcggtatcggagggaaccctaaccctaaccct

SEQ ID NO: 101 - DNA, 100 bp, synthetic construct
  source 1..100: mol_type = other DNA
caccattaattaagaattccgatcgggcgcgcctgattgctgtacgcagaagttagaagccacaaggatcggtatcggagggaccctaaccctaacccta

SEQ ID NO: 102 - DNA, 100 bp, synthetic construct
  source 1..100: mol_type = other DNA
caccattaattaagaattccgatcgggcgcgcctgattgctgtacgcaggtgagcacacgagtatgacaaactatcggagggccctaaccctaaccctaa

SEQ ID NO: 103 - DNA, 100 bp, synthetic construct
  source 1..100: mol_type = other DNA
caccattaattaagaattccgatcgggcgcgcctgattgctgtacgcaggtgagcacacgagtatgacaaactatcggagggcctaaccctaaccctaac

SEQ ID NO: 104 - DNA, 100 bp, synthetic construct
  source 1..100: mol_type = other DNA
caccattaattaagaattccgatcgggcgcgcctgattgctgtacgcaggtgagcacacgagtatgacaaactatcggagggctaaccctaaccctaacc

SEQ ID NO: 105 - DNA, 100 bp, synthetic construct
  source 1..100: mol_type = other DNA
caccattaattaagaattccgatcgggcgcgcctgattgctgtacgcaggtgagcacacgagtatgacaaactatcggagggtaaccctaaccctaaccc

SEQ ID NO: 106 - DNA, 100 bp, synthetic construct
  source 1..100: mol_type = other DNA
caccattaattaagaattccgatcgggcgcgcctgattgctgtacgcaggtgagcacacgagtatgacaaactatcggagggaaccctaaccctaaccct

SEQ ID NO: 107 - DNA, 100 bp, synthetic construct
  source 1..100: mol_type = other DNA
caccattaattaagaattccgatcgggcgcgcctgattgctgtacgcaggtgagcacacgagtatgacaaactatcggagggaccctaaccctaacccta

SEQ ID NO: 108 - DNA, 100 bp, synthetic construct
  source 1..100: mol_type = other DNA
caccattaattaagaattccgatcgggcgcgcctgattgctgtacgcaagatcacatgaggctcggactgtatatcggagggccctaaccctaaccctaa

SEQ ID NO: 109 - DNA, 100 bp, synthetic construct
  source 1..100: mol_type = other DNA
caccattaattaagaattccgatcgggcgcgcctgattgctgtacgcaagatcacatgaggctcggactgtatatcggagggcctaaccctaaccctaac

SEQ ID NO: 110 - DNA, 100 bp, synthetic construct
  source 1..100: mol_type = other DNA
caccattaattaagaattccgatcgggcgcgcctgattgctgtacgcaagatcacatgaggctcggactgtatatcggagggctaaccctaaccctaacc

SEQ ID NO: 111 - DNA, 100 bp, synthetic construct
  source 1..100: mol_type = other DNA
caccattaattaagaattccgatcgggcgcgcctgattgctgtacgcaagatcacatgaggctcggactgtatatcggagggtaaccctaaccctaaccc

SEQ ID NO: 112 - DNA, 100 bp, synthetic construct
  source 1..100: mol_type = other DNA
caccattaattaagaattccgatcgggcgcgcctgattgctgtacgcaagatcacatgaggctcggactgtatatcggagggaaccctaaccctaaccct

SEQ ID NO: 113 - DNA, 100 bp, synthetic construct
  source 1..100: mol_type = other DNA
caccattaattaagaattccgatcgggcgcgcctgattgctgtacgcaagatcacatgaggctcggactgtatatcggagggaccctaaccctaacccta

SEQ ID NO: 114 - DNA, 100 bp, synthetic construct
  source 1..100: mol_type = other DNA
caccattaattaagaattccgatcgggcgcgcctgattgctgtacgcaacactccattcgtaggatctcggttatcggagggccctaaccctaaccctaa

SEQ ID NO: 115 - DNA, 100 bp, synthetic construct
  source 1..100: mol_type = other DNA
caccattaattaagaattccgatcgggcgcgcctgattgctgtacgcaacactccattcgtaggatctcggttatcggagggcctaaccctaaccctaac

SEQ ID NO: 116 - DNA, 100 bp, synthetic construct
  source 1..100: mol_type = other DNA
caccattaattaagaattccgatcgggcgcgcctgattgctgtacgcaacactccattcgtaggatctcggttatcggagggctaaccctaaccctaacc

SEQ ID NO: 117 - DNA, 100 bp, synthetic construct
  source 1..100: mol_type = other DNA
caccattaattaagaattccgatcgggcgcgcctgattgctgtacgcaacactccattcgtaggatctcggttatcggagggtaaccctaaccctaaccc

SEQ ID NO: 118 - DNA, 100 bp, synthetic construct
  source 1..100: mol_type = other DNA
caccattaattaagaattccgatcgggcgcgcctgattgctgtacgcaacactccattcgtaggatctcggttatcggagggaaccctaaccctaaccct

SEQ ID NO: 119 - DNA, 100 bp, synthetic construct
  source 1..100: mol_type = other DNA
caccattaattaagaattccgatcgggcgcgcctgattgctgtacgcaacactccattcgtaggatctcggttatcggagggaccctaaccctaacccta

SEQ ID NO: 120 - DNA, 100 bp, synthetic construct
  source 1..100: mol_type = other DNA
caccattaattaagaattccgatcgggcgcgcctgattgctgtacgcagtcggtatggaagacagtcagctatatcggagggccctaaccctaaccctaa

SEQ ID NO: 121 - DNA, 100 bp, synthetic construct
  source 1..100: mol_type = other DNA
caccattaattaagaattccgatcgggcgcgcctgattgctgtacgcagtcggtatggaagacagtcagctatatcggagggcctaaccctaaccctaac

SEQ ID NO: 122 - DNA, 100 bp, synthetic construct
  source 1..100: mol_type = other DNA
caccattaattaagaattccgatcgggcgcgcctgattgctgtacgcagtcggtatggaagacagtcagctatatcggagggctaaccctaaccctaacc

SEQ ID NO: 123 - DNA, 100 bp, synthetic construct
  source 1..100: mol_type = other DNA
caccattaattaagaattccgatcgggcgcgcctgattgctgtacgcagtcggtatggaagacagtcagctatatcggagggtaaccctaaccctaaccc

SEQ ID NO: 124 - DNA, 100 bp, synthetic construct
  source 1..100: mol_type = other DNA
caccattaattaagaattccgatcgggcgcgcctgattgctgtacgcagtcggtatggaagacagtcagctatatcggagggaaccctaaccctaaccct

SEQ ID NO: 125 - DNA, 100 bp, synthetic construct
  source 1..100: mol_type = other DNA
caccattaattaagaattccgatcgggcgcgcctgattgctgtacgcagtcggtatggaagacagtcagctatatcggagggaccctaaccctaacccta

SEQ ID NO: 126 - DNA, 99 bp, synthetic construct
  source 1..99: mol_type = other DNA
gagtccaccatgaattccactgacaatgtaactgattgctgtacgcaaagcgttgaaacctttgtcctctctatcggagggccctaaccctaaccctaa

SEQ ID NO: 127 - DNA, 99 bp, synthetic construct
  source 1..99: mol_type = other DNA
gagtccaccatgaattccactgacaatgtaactgattgctgtacgcaaagcgttgaaacctttgtcctctctatcggagggcctaaccctaaccctaac

SEQ ID NO: 128 - DNA, 99 bp, synthetic construct
  source 1..99: mol_type = other DNA
gagtccaccatgaattccactgacaatgtaactgattgctgtacgcaaagcgttgaaacctttgtcctctctatcggagggctaaccctaaccctaacc

SEQ ID NO: 129 - DNA, 99 bp, synthetic construct
  source 1..99: mol_type = other DNA
gagtccaccatgaattccactgacaatgtaactgattgctgtacgcaaagcgttgaaacctttgtcctctctatcggagggtaaccctaaccctaaccc

SEQ ID NO: 130 - DNA, 99 bp, synthetic construct
  source 1..99: mol_type = other DNA
gagtccaccatgaattccactgacaatgtaactgattgctgtacgcaaagcgttgaaacctttgtcctctctatcggagggaaccctaaccctaaccct

SEQ ID NO: 131 - DNA, 99 bp, synthetic construct
  source 1..99: mol_type = other DNA
gagtccaccatgaattccactgacaatgtaactgattgctgtacgcaaagcgttgaaacctttgtcctctctatcggagggaccctaaccctaacccta

SEQ ID NO: 132 - DNA, 99 bp, synthetic construct
  source 1..99: mol_type = other DNA
gagtccaccatgaattccactgacaatgtaactgattgctgtacgcatcacacgagtatggaagtcgttcttatcggagggccctaaccctaaccctaa

SEQ ID NO: 133 - DNA, 99 bp, synthetic construct
  source 1..99: mol_type = other DNA
gagtccaccatgaattccactgacaatgtaactgattgctgtacgcatcacacgagtatggaagtcgttcttatcggagggcctaaccctaaccctaac

SEQ ID NO: 134 - DNA, 99 bp, synthetic construct
  source 1..99: mol_type = other DNA
gagtccaccatgaattccactgacaatgtaactgattgctgtacgcatcacacgagtatggaagtcgttcttatcggagggctaaccctaaccctaacc

SEQ ID NO: 135 - DNA, 99 bp, synthetic construct
  source 1..99: mol_type = other DNA
gagtccaccatgaattccactgacaatgtaactgattgctgtacgcatcacacgagtatggaagtcgttcttatcggagggtaaccctaaccctaaccc

SEQ ID NO: 136 - DNA, 99 bp, synthetic construct
  source 1..99: mol_type = other DNA
gagtccaccatgaattccactgacaatgtaactgattgctgtacgcatcacacgagtatggaagtcgttcttatcggagggaaccctaaccctaaccct

SEQ ID NO: 137 - DNA, 99 bp, synthetic construct
  source 1..99: mol_type = other DNA
gagtccaccatgaattccactgacaatgtaactgattgctgtacgcatcacacgagtatggaagtcgttcttatcggagggaccctaaccctaacccta

SEQ ID NO: 138 - DNA, 99 bp, synthetic construct
  source 1..99: mol_type = other DNA
gagtccaccatgaattcggcgcgccatgtaactgattgctgtacgcaagtacgaaccactgtcagttgacgtatcggagggccctaaccctaaccctaa

SEQ ID NO: 139 - DNA, 99 bp, synthetic construct
  source 1..99: mol_type = other DNA
gagtccaccatgaattcggcgcgccatgtaactgattgctgtacgcaagtacgaaccactgtcagttgacgtatcggagggcctaaccctaaccctaac

SEQ ID NO: 140 - DNA, 99 bp, synthetic construct
  source 1..99: mol_type = other DNA
gagtccaccatgaattcggcgcgccatgtaactgattgctgtacgcaagtacgaaccactgtcagttgacgtatcggagggctaaccctaaccctaacc

SEQ ID NO: 141 - DNA, 99 bp, synthetic construct
  source 1..99: mol_type = other DNA
gagtccaccatgaattcggcgcgccatgtaactgattgctgtacgcaagtacgaaccactgtcagttgacgtatcggagggtaaccctaaccctaaccc

SEQ ID NO: 142 - DNA, 99 bp, synthetic construct
  source 1..99: mol_type = other DNA
gagtccaccatgaattcggcgcgccatgtaactgattgctgtacgcaagtacgaaccactgtcagttgacgtatcggagggaaccctaaccctaaccct

SEQ ID NO: 143 - DNA, 99 bp, synthetic construct
  source 1..99: mol_type = other DNA
gagtccaccatgaattcggcgcgccatgtaactgattgctgtacgcaagtacgaaccactgtcagttgacgtatcggagggaccctaaccctaacccta

SEQ ID NO: 144 - skipped

SEQ ID NO: 145 - DNA, 94 bp, synthetic construct
  source 1..94: mol_type = other DNA
ccctccgatagagaggacaaaggtttcaacgctttgcgtacagcaatcagttacattgtcagtggaattcatggtggactctatgaccgttcag

SEQ ID NO: 146 - DNA, 99 bp, synthetic construct; identical to SEQ ID NO: 126
  source 1..99: mol_type = other DNA

SEQ ID NO: 147 - DNA, 99 bp, synthetic construct; identical to SEQ ID NO: 127
  source 1..99: mol_type = other DNA

SEQ ID NO: 148 - DNA, 99 bp, synthetic construct; identical to SEQ ID NO: 128
  source 1..99: mol_type = other DNA

SEQ ID NO: 149 - DNA, 99 bp, synthetic construct; identical to SEQ ID NO: 129
  source 1..99: mol_type = other DNA

SEQ ID NO: 150 - DNA, 99 bp, synthetic construct; identical to SEQ ID NO: 130
  source 1..99: mol_type = other DNA

SEQ ID NO: 151 - DNA, 99 bp, synthetic construct; identical to SEQ ID NO: 131
  source 1..99: mol_type = other DNA

SEQ ID NO: 152 - DNA, 94 bp, synthetic construct
  source 1..94: mol_type = other DNA
ccctccgatatccgattctgcttctttctacctgtgcgtacagcaatcagttacattgtcagtggaattcatggtggactctatgaccgttcag

SEQ ID NO: 153 - DNA, 99 bp, synthetic construct
  source 1..99: mol_type = other DNA
gagtccaccatgaattccactgacaatgtaactgattgctgtacgcacaggtagaaagaagcagaatcggatatcggagggccctaaccctaaccctaa

SEQ ID NO: 154 - DNA, 99 bp, synthetic construct; identical to SEQ ID NO: 127
  source 1..99: mol_type = other DNA

SEQ ID NO: 155 - DNA, 99 bp, synthetic construct; identical to SEQ ID NO: 128
  source 1..99: mol_type = other DNA

SEQ ID NO: 156 - DNA, 99 bp, synthetic construct; identical to SEQ ID NO: 129
  source 1..99: mol_type = other DNA

SEQ ID NO: 157 - DNA, 99 bp, synthetic construct; identical to SEQ ID NO: 130
  source 1..99: mol_type = other DNA

SEQ ID NO: 158 - DNA, 99 bp, synthetic construct; identical to SEQ ID NO: 131
  source 1..99: mol_type = other DNA

SEQ ID NO: 159 - DNA, 94 bp, synthetic construct
  source 1..94: mol_type = other DNA
ccctccgataagaacgacttccatactcgtgtgatgcgtacagcaatcagttacattgtcagtggaattcatggtggactctatgaccgttcag

SEQ ID NO: 160 - DNA, 99 bp, synthetic construct; identical to SEQ ID NO: 132
  source 1..99: mol_type = other DNA

SEQ ID NO: 161 - DNA, 99 bp, synthetic construct; identical to SEQ ID NO: 133
  source 1..99: mol_type = other DNA

SEQ ID NO: 162 - DNA, 99 bp, synthetic construct; identical to SEQ ID NO: 134
  source 1..99: mol_type = other DNA

SEQ ID NO: 163 - DNA, 99 bp, synthetic construct; identical to SEQ ID NO: 135
  source 1..99: mol_type = other DNA

SEQ ID NO: 164 - DNA, 99 bp, synthetic construct; identical to SEQ ID NO: 136
  source 1..99: mol_type = other DNA

SEQ ID NO: 165 - DNA, 99 bp, synthetic construct; identical to SEQ ID NO: 137
  source 1..99: mol_type = other DNA

SEQ ID NO: 166 - DNA, 98 bp, synthetic construct
  source 1..98: mol_type = other DNA
ccctccgatacgtcaactgacagtggttcgtacttgcgtacagcaatcagttacatggcgcgccgaattcatggtggactctatgaccgttcagagct

SEQ ID NO: 167 - DNA, 99 bp, synthetic construct; identical to SEQ ID NO: 138
  source 1..99: mol_type = other DNA

SEQ ID NO: 168 - DNA, 99 bp, synthetic construct; identical to SEQ ID NO: 139
  source 1..99: mol_type = other DNA

SEQ ID NO: 169 - DNA, 99 bp, synthetic construct; identical to SEQ ID NO: 140
  source 1..99: mol_type = other DNA

SEQ ID NO: 170 - DNA, 99 bp, synthetic construct; identical to SEQ ID NO: 141
  source 1..99: mol_type = other DNA

SEQ ID NO: 171 - DNA, 99 bp, synthetic construct; identical to SEQ ID NO: 142
  source 1..99: mol_type = other DNA

SEQ ID NO: 172 - DNA, 99 bp, synthetic construct; identical to SEQ ID NO: 143
  source 1..99: mol_type = other DNA

SEQ ID NO: 173 - DNA, 98 bp, synthetic construct
  source 1..98: mol_type = other DNA
ccctccgatagttcctcgtgcagtgtcaagagattgcgtacagcaatcagttacatggcgcgccgaattcatggtggactctatgaccgttcagagct

SEQ ID NO: 174 - DNA, 99 bp, synthetic construct
  source 1..99: mol_type = other DNA
gagtccaccatgaattcggcgcgccatgtaactgattgctgtacgcaatctcttgacactgcacgaggaactatcggagggccctaaccctaaccctaa

SEQ ID NO: 175 - DNA, 99 bp, synthetic construct
  source 1..99: mol_type = other DNA
gagtccaccatgaattcggcgcgccatgtaactgattgctgtacgcaatctcttgacactgcacgaggaactatcggagggcctaaccctaaccctaac

SEQ ID NO: 176 - DNA, 99 bp, synthetic construct
  source 1..99: mol_type = other DNA
gagtccaccatgaattcggcgcgccatgtaactgattgctgtacgcaatctcttgacactgcacgaggaactatcggagggctaaccctaaccctaacc

SEQ ID NO: 177 - DNA, 99 bp, synthetic construct
  source 1..99: mol_type = other DNA
gagtccaccatgaattcggcgcgccatgtaactgattgctgtacgcaatctcttgacactgcacgaggaactatcggagggtaaccctaaccctaaccc

SEQ ID NO: 178 - DNA, 99 bp, synthetic construct
  source 1..99: mol_type = other DNA
gagtccaccatgaattcggcgcgccatgtaactgattgctgtacgcaatctcttgacactgcacgaggaactatcggagggaaccctaaccctaaccct

SEQ ID NO: 179 - DNA, 99 bp, synthetic construct
  source 1..99: mol_type = other DNA
gagtccaccatgaattcggcgcgccatgtaactgattgctgtacgcaatctcttgacactgcacgaggaactatcggagggaccctaaccctaacccta

SEQ ID NO: 180 - DNA, 26 bp, synthetic construct
  source 1..26: mol_type = other DNA
gctacatgctcctgttgttaggaggg

SEQ ID NO: 181 - DNA, 44 bp, synthetic construct
  source 1..44: mol_type = other DNA
gctacatgctcctgttgttaggagggccctaaccctaaccctaa

SEQ ID NO: 182 - DNA, 44 bp, synthetic construct
  source 1..44: mol_type = other DNA
gctacatgctcctgttgttaggagggcctaaccctaaccctaac

SEQ ID NO: 183 - DNA, 44 bp, synthetic construct
  source 1..44: mol_type = other DNA
gctacatgctcctgttgttaggagggctaaccctaaccctaacc

SEQ ID NO: 184 - DNA, 44 bp, synthetic construct
  source 1..44: mol_type = other DNA
gctacatgctcctgttgttaggagggtaaccctaaccctaaccc

SEQ ID NO: 185 - DNA, 44 bp, synthetic construct
  source 1..44: mol_type = other DNA
gctacatgctcctgttgttaggagggaaccctaaccctaaccct

SEQ ID NO: 186 - DNA, 44 bp, synthetic construct
  source 1..44: mol_type = other DNA
gctacatgctcctgttgttaggagggaccctaaccctaacccta

SEQ ID NO: 187 - DNA, 98 bp, synthetic construct
  source 1..98: mol_type = other DNA
ccctcctaacaacaggagcatgtagcnnnyrnnnyrnnnyrnnncctggtaactgggacacaagactcgcctcgtcgactgacagatgtcacggagca

SEQ ID NO: 188 - DNA, 98 bp, synthetic construct
  source 1..98: mol_type = other DNA
ccctcctaacaacaggagcatgtagcnnnyrnnnyrnnnyrnnnttgcgtcctgttacgagaactcatgcctcgtcgactgacagatgtcacggagca

SEQ ID NO: 189 - DNA, 98 bp, synthetic construct
  source 1..98: mol_type = other DNA
ccctcctaacaacaggagcatgtagcnnnyrnnnyrnnnyrnnngagcctctcattgtccgttctctagcctcgtcgactgacagatgtcacggagca

SEQ ID NO: 190 - DNA, 98 bp, synthetic construct
  source 1..98: mol_type = other DNA
ccctcctaacaacaggagcatgtagcnnnyrnnnyrnnnyrnnnaccactgccatgtatcaaagtacggcctcgtcgactgacagatgtcacggagca

SEQ ID NO: 191 - DNA, 98 bp, synthetic construct
  source 1..98: mol_type = other DNA
ccctcctaacaacaggagcatgtagcnnnyrnnnyrnnnyrnnncatacagcgactacgcattctcatgcctcgtcgactgacagatgtcacggagca

SEQ ID NO: 192 - DNA, 98 bp, synthetic construct
  source 1..98: mol_type = other DNA
ccctcctaacaacaggagcatgtagcnnnyrnnnyrnnnyrnnncgacggttagattcacctcttacagcctcgtcgactgacagatgtcacggagca

SEQ ID NO: 193 - DNA, 30 bp, synthetic construct
  source 1..30: mol_type = other DNA
tgctccgtgacatctgtcagtcgacgaggc

(19) United States
(12) Patent Application Publication
(10) Pub. No.: US 2026/0347923 A1
(43) Pub. Date: Nov. 5, 2026

(54) METHODS FOR MEASURING TELOMERE LENGTH

(71) Applicants: The Johns Hopkins University, Baltimore, MD (US); The Regents of the University of California, Oakland, CA (US)

(72) Inventors: Kayarash Karimian, Baltimore, MD (US); Carol W. Greider, Baltimore, MD (US)

(21) Appl. No.: 19/107,328

(22) PCT Filed: Sep. 1, 2023
(86) PCT No.: PCT/US2023/073375

§ 371 Date: Feb. 27, 2025

Related U.S. Application Data
(60) Provisional application No. 63/403,159, filed on Sep. 1, 2022.
(60) Provisional application No. 63/463,598, filed on May 3, 2023.

(51) Int. Cl. C12Q 1/686; C12Q 1/25; C12Q 1/48; C12Q 1/6806; C12Q 1/6813; C12Q 1/6855; C12Q 1/6869; C12Q 1/6876; C40B 70/00

(52) U.S. Cl. CPC C12Q 1/686; C12Q 1/25; C12Q 1/48; C12Q 1/6806; C12Q 1/6813; C12Q 1/6855; C12Q 1/6869; C12Q 1/6876; C12Y 207/07; C12Y 605/01002; C40B 70/00

(57) ABSTRACT

Provided herein are compositions and methods for determining telomere length. In some embodiments, methods include attaching a telomere tagging probe to a 3′ end of the telomere to generate a tagged telomere sequence and use of a splint oligonucleotide comprising a nucleic acid sequence that specifically binds to at least a portion of the telomere tagging probe and to at least a portion of the telomere.

### CROSS-REFERENCE TO RELATED APPLICATIONS

This application claims priority to U.S. Provisional Patent Application Nos. 63/403,159, filed on Sep. 1, 2022 and 63/463,598, filed on May 3, 2023, which are incorporated herein by reference in their entireties.

### FEDERALLY SPONSORED RESEARCH OR DEVELOPMENT

This invention was made with government support under grant CA209974 awarded by the National Institutes of Health. The government has certain rights in the invention.

### SEQUENCE LISTING

This application contains a Sequence Listing that has been submitted electronically as an XML file named “44807-0447WO1_ST26_SL.XML.” The XML file, created on Aug. 31, 2023, is 185,223 bytes in size. The material in the XML file is hereby incorporated by reference in its entirety.

### TECHNICAL FIELD

The present disclosure relates to the field of biotechnology, and more specifically, to measurement of mammalian telomere lengths.

### BACKGROUND

Telomeres are the end of eukaryotic chromosomes and shorten with time. Shortened telomeres cause disease in patients with short telomere syndromes. Cancer cells must activate telomerase to maintain telomeres to overcome senescence or apoptosis due to critically short telomeres.

Telomere length is often measured by Southern Blot (invented in 1973). This method measures the length of all the telomeres in the cell and does not give quantitative information or information about individual telomere lengths. A quantitative PCR method was developed however it has many problems with reproducibility across different labs. The Single Telomere Length Analysis (STELA) method and the related Telomere Shortest Length Assay (TeSLA) targets individual telomeres, but the use of high cycles of PCR generates bias for shorter products. Additionally, the use of Southern Blotting in STELA and TeSLA makes these techniques laborious and low-throughput. Currently, Telomere Flow FISH is the accepted clinical method for measuring telomere length. This method is highly reproducible, and can report on a specific cell type, however it requires fresh blood samples and is not able to measure archival samples or tissues other than blood. The method uses an average of the telomeres without the ability to report the individual lengths of telomeres. Thus, there are currently no methods that provide an accurate, high throughput, and simple approach for quantifying telomere length.

### SUMMARY

The present disclosure is based in part on the discovery of technology that allows for accurate high-throughput measurement of mammalian telomere lengths. Without wishing to be bound by any theory, the methods disclosed herein include tagging of the ends of telomeres to mark their natural ends, therefore providing an accurate, high-throughput, and simple approach for quantifying telomere length.

Provided herein are methods for determining a telomere length of a DNA molecule from a biological sample, the method comprising: (a) providing a telomere tagging probe comprising a biotin adapter; (b) providing a splint oligonucleotide, wherein the splint oligonucleotide specifically binds to at least a portion of a telomere on the DNA molecule and to at least a portion of the telomere tagging probe; (c) attaching the telomere tagging probe to the telomere to generate a tagged telomere sequence; (d) contacting the tagged telomere sequence with a streptavidin-functionalized bead, wherein the biotin adapter of the tagged telomere sequence binds the streptavidin-functionalized bead; (e) recovering the tagged telomere sequence; and (f) analyzing the tagged telomere sequence, thereby determining the telomere length of the DNA molecule.

In some embodiments, the splint oligonucleotide is not blocked at its 3′ end.

In some embodiments, the attaching step (c) comprises ligating the telomere tagging probe to the 3′ end of the telomere. In some embodiments, the ligating comprises cycling ligation. In some embodiments, the ligating comprises a Taq DNA ligase.

In some embodiments, the recovering comprises using a magnet to separate the tagged telomere sequence bound to the streptavidin-functionalized bead. In some embodiments, the recovering further comprises releasing the tagged telomere sequence from the streptavidin-functionalized bead using a restriction enzyme, wherein the restriction enzyme cleaves the tagged telomere sequence at a restriction enzyme-specific site.

In some embodiments, the analyzing comprises sequencing the released tagged telomere sequence. In some embodiments, the sequencing comprises nanopore sequencing.

Also provided herein are methods for determining a telomere length of a DNA molecule from a biological sample, the method comprising: (a) providing a telomere tagging probe, wherein the telomere tagging probe comprises a unique molecular identifier (UMI) sequence; (b) providing a splint oligonucleotide, wherein the splint oligonucleotide specifically binds to at least a portion of a telomere on the DNA molecule and to at least a portion of the telomere tagging probe; (c) attaching the telomere tagging probe to the telomere to generate a tagged telomere sequence; (d) contacting the tagged telomere sequence with a forward primer and a reverse primer, wherein the forward primer binds to a subtelomere of the telomere, and the reverse primer binds to at least a portion of the telomere tagging probe; (e) amplifying the tagged telomere sequence to generate an amplified tagged telomere sequence; and (f) analyzing the amplified tagged telomere sequence, thereby determining the telomere length of the DNA molecule.

In some embodiments, the splint oligonucleotide is blocked at its 3′ end. In some embodiments, the splint oligonucleotide is not blocked at its 3′ end.

In some embodiments, the attaching step (c) comprises ligating the telomere tagging probe to the 3′ end of the telomere. In some embodiments, the ligating comprises cycling ligation. In some embodiments, the ligating comprises a Taq ligase.

In some embodiments, the amplification comprises PCR amplification. In some embodiments, the amplification comprises 20 rounds of PCR amplification. In some embodiments, the analyzing comprises sequencing the tagged telomere sequence. In some embodiments, the sequencing comprises long read sequencing.

In some embodiments, the telomere tagging probe further comprises a sample barcode sequence that is associated with the biological sample. In some embodiments, the biological sample comprises a blood sample. In some embodiments, the biological sample comprises a tissue sample.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an exemplary workflow of a tagging and sequencing method described herein as the Direct Telomere Profiling method. Here, a Telomere Tag (“TeloTag”) is biotinylated to allow enrichment of telomeres, which are then sequenced with Oxford Nanopore Technology (ONT) platforms.

FIG. 2 shows an exemplary workflow of a tagging and sequencing method described herein as the Amplified Telomere Profiling method. This method uses PCR amplification from telomeres tagged with TeloTags containing UMI barcodes followed by PacBio sequencing.

FIG. 3A shows telomere enrichments captures many reads by the Direct Telomere Profiling method.

FIG. 3B shows a portion of a reference genome used to assess Nanopore telomere profiling to accurately measure telomere length.

FIG. 4 shows using Direct Telomere Profiling method and nanopore sequencing on blood samples can recapitulate results from Southern Blotting.

FIG. 5A shows length bias of nanopore sequencing does not impact telomere length measured by Direct Telomere Profiling method.

FIG. 5B shows that enrichment captures ~20% of input telomeres.

FIG. 6A shows telomere profiling using the Direct Telomere Profiling method is highly reproducible.

FIG. 6B shows that samples processed at two different laboratories reproduce the same result, showing the reproducibility of the method.

FIG. 7 shows telomere profiling showing decrease of telomere length with age.

FIG. 8A shows telomere length amongst general population (e.g., healthy donors).

FIG. 8B shows graphs wherein the lines are the derived 10^{th}, 50^{th}, and 90^{th} percentile of telomere length based on the nanopore profiling method. The dots represent identical samples for both right and left panels. Right panel shows results from the clinically accepted Flowfish method. In comparison, left panel shows that the telomere length calculated by the nanopore profiling method falls on relatively the same percentile calculations as the ones done for Flowfish. That is, patients below the 10^{th} percentile of telomere length that could have telomere disease can be screened and potentially diagnosed.

FIG. 8C shows the predictive value from the nanopore profiling method. Samples were chosen just based on the nanopore value and then a prospective southern blot is shown in the left panel.

The Southern blot shows that, as expected, the telomere lengths of those samples show up in decreasing length order.

FIG. 9 shows chromosome end-specific telomere length on a subset of telomeres that align with high confidence.

FIGS. 10A-10B show chromosome specific telomere assignment corrected for age across many individuals. Based on the findings, certain chromosomes such as 17p, 19p, 17q, 22q are predicted to be risk factors for telomere disease as they are generally shorter than all other telomeres across many humans.

FIG. 11 shows an example of chromosome specific assignment of a sample from a short telomere patient. The results in the left panel show a bimodal distribution, wherein the longer “mode” represent telomeres inherited from the healthy parent, while the lower “mode” represents telomeres inherited from parent with telomere disease. The right panels show distribution of telomere length in the normal human population.

FIGS. 12A-12B show telomere length differences on chromosomes are set at birth based on analysis of cord blood.

### DETAILED DESCRIPTION

The present disclosure is based in part on the discovery of technology that allows for accurate high-throughput measurement of mammalian telomere lengths. In some embodiments, a method for tagging and sequencing a telomere can include tagging of the ends of telomeres to mark their natural ends, therefore providing an accurate, high-throughput, and simple approach for quantifying telomere length. In some embodiments, a biotinylated Telomere Tag (TeloTag) is ligated to the 3′ end of a telomere to allow enrichment of telomeres, which are then sequenced with high throughput sequencing protocols to measure telomere lengths. In some embodiments, tagging and sequencing of a telomere can be performed by using PCR amplification for amplification of the telomeres tagged with TeloTags containing UMI barcodes followed by a sequencing protocol.

Provided herein are methods for determining a telomere length of a DNA molecule from a biological sample that include (a) providing a telomere tagging probe comprising a biotin adapter; (b) providing a splint oligonucleotide, wherein the splint oligonucleotide specifically binds to at least a portion of a telomere on the DNA molecule and to at least a portion of the telomere tagging probe; (c) attaching the telomere tagging probe to the telomere to generate a tagged telomere sequence; (d) contacting the tagged telomere sequence with a streptavidin-functionalized bead, wherein the biotin adapter of the tagged telomere sequence binds the streptavidin-functionalized bead; (e) recovering the tagged telomere sequence; and (f) analyzing the tagged telomere sequence, thereby determining the telomere length of the DNA molecule.

Also provided herein are methods for determining a telomere length of a DNA molecule from a biological sample that include (a) providing a telomere tagging probe, wherein the telomere tagging probe comprises a unique molecular identifier (UMI) sequence; (b) providing a splint oligonucleotide, wherein the splint oligonucleotide specifically binds to at least a portion of a telomere on the DNA molecule and to at least a portion of the telomere tagging probe; (c) attaching the telomere tagging probe to the telomere to generate a tagged telomere sequence; (d) contacting the tagged telomere sequence with a forward primer and a reverse primer, wherein the forward primer binds to a subtelomere of the telomere, and the reverse primer binds to at least a portion of the telomere tagging probe; (e) amplifying the tagged telomere sequence to generate an amplified tagged telomere sequence; and (f) analyzing the amplified tagged telomere sequence, thereby determining the telomere length of the DNA molecule.

Various non-limiting aspects of these methods are described herein, and can be used in any combination without limitation. Additional aspects of various components of the methods described herein are known in the art.

It must be noted that, as used in the specification and the appended claims, the singular forms “a,” “an” and “the” include plural referents unless the context clearly dictates otherwise.

As used herein, an “adaptor,” an “adapter,” and a “tag” are terms that are used interchangeably, and refer to species that can be coupled to a polynucleotide sequence (e.g., in a process referred to as “tagging”) using any one of many different techniques including, but not limited to, ligation, hybridization, and tagmentation. In some embodiments, adaptors can also be nucleic acid sequences that add a function, e.g., spacer sequences, primer sequences/sites, barcode sequences, or unique molecular identifier (UMI) sequences.

As used herein, the term “barcode” refers to a label, or identifier, that conveys or is capable of conveying information (e.g., information about an analyte in a sample). A barcode can be part of an analyte, or independent of an analyte. In some embodiments, a barcode can be attached to an analyte. In some embodiments, a particular barcode can be unique relative to other barcodes. In some embodiments, barcodes can have a variety of different formats. For example, barcodes can include non-random, semi-random, and/or random nucleic acids, and synthetic nucleic acids. In some embodiments, a barcode can be attached to an analyte or to another moiety or structure in a reversible or irreversible manner. In some embodiments, a barcode can be added to, for example, a fragment of a deoxyribonucleic acid (DNA) or ribonucleic acid (RNA) sample before or during sequencing of the sample. In some embodiments, barcodes can allow for identification and/or quantification of individual sequencing-reads. In some embodiments, a barcode can refer to a unique molecular identifier (UMI) and the terms “barcode” and “UID” and “UMI” can be used interchangeably.

As used herein, the term “biological sample” refers to a sample obtained from a subject for analysis using any of a variety of techniques, and generally includes cells and/or other biological material from the subject. In some embodiments, biological samples include, but are not limited to, plasma, serum, blood, tissue, tumor sample, stool, sputum, saliva, urine, sweat, tears, ascites, bronchoaveolar lavage, semen, archeologic specimens, and forensic samples. In some embodiments, the biological sample is a solid biological sample, e.g., a tumor sample. In some embodiments, the solid biological sample is processed. The solid biological sample may be processed by fixation in a formalin solution, followed by embedding in paraffin (e.g., is a FFPE sample). Processing can alternatively comprise freezing of the sample prior to conducting the probe-based assay. In some embodiments, the sample is neither fixed nor frozen. The unfixed, unfrozen sample can be, by way of example only, stored in a storage solution configured for the preservation of nucleic acid.

In some embodiments, the biological sample is a liquid biological sample. Liquid biological samples include, but are not limited to, plasma, serum, blood, sputum, saliva, urine, sweat, tears, ascites, bronchoaveolar lavage, and semen. In some embodiments, the liquid biological sample is cell-free or substantially cell-free. In some embodiments, the biological sample is a plasma or serum sample. In some embodiments, the liquid biological sample is a whole blood sample. In some embodiments, the liquid biological sample includes peripheral mononuclear blood cells.

As used herein, the term “nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. In some embodiments, nucleic acid modifications can also include a blocking modification comprising a 3′ end modification (e.g., a 3′ dideoxy C (3′ddC), 3′ddG, 3′ddA, 3′ddT, 3′ inverted dT, 3′ C3 spacer, 3′ amino, 3′ biotinylation, or 3′ phosphorylation). The terms “polynucleotides,” “nucleic acid,” and “oligonucleotides” can be used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, a “primer” generally refers to a polynucleotide molecule comprising a nucleotide sequence (e.g., an oligonucleotide), generally with a free 3′-OH group, that hybridizes with a template sequence (such as a target polynucleotide, or a primer extension product) and is capable of promoting polymerization of a polynucleotide complementary to the template.

### Telomeres and Telomere Length

A telomere is a region of repetitive nucleotide sequences associated with specialized proteins at the ends of linear chromosomes. Telomeres are a genetic feature most commonly found in eukaryotes, and are known to protect the terminal regions of chromosomal DNA from progressive degradation and ensure the integrity of linear chromosomes by preventing DNA repair systems from mistaking the very ends of the DNA strand for a double strand break. Telomeres are also critical for maintaining genomic integrity and may be factors for age-related diseases (e.g., Bone marrow failure, pulmonary fibrosis, immunodeficiency and other related Telomere Syndromes). Laboratory studies and clinical observations have shown that telomere dysfunction or shortening is commonly acquired due process of cellular aging and tumor development, wherein observational studies have found shortened telomeres in many types of experimental cancers (e.g., squamous cell carcinomas of the skin and upper aerodigestive tract, myelodysplasia, and acute myeloid leukemia). Despite having short telomeres, cancer cells typically have activated telomerase that will maintain telomeres. In some cases, long telomeres inherited in families predispose to cancer.

Most vertebrate telomeric DNA consists of long (TTAGGG)n repeats of variable length, often around 3-20 kb. As used herein, a “subtelomere” refers to a segment of DNA between telomeric caps and coding regions of DNA. In vertebrates, each chromosome has two subtelomeres immediately adjacent to the long (TTAGGG)n repeats at each chromosome end. In some embodiments, subtelomeres are considered to be the most distal (farthest from the centromere) region of unique DNA on a chromosome, and they are unusually dynamic and variable mosaics of highly repeated blocks of sequence.

Telomere length varies greatly between species, from approximately 300 base pairs in yeast to many kilobases in humans, and usually is composed of arrays of guanine-rich, six- to eight-base-pair-long repeats. Eukaryotic telomeres normally terminate with 3′ single-stranded-DNA overhang, which is essential for telomere maintenance and capping.

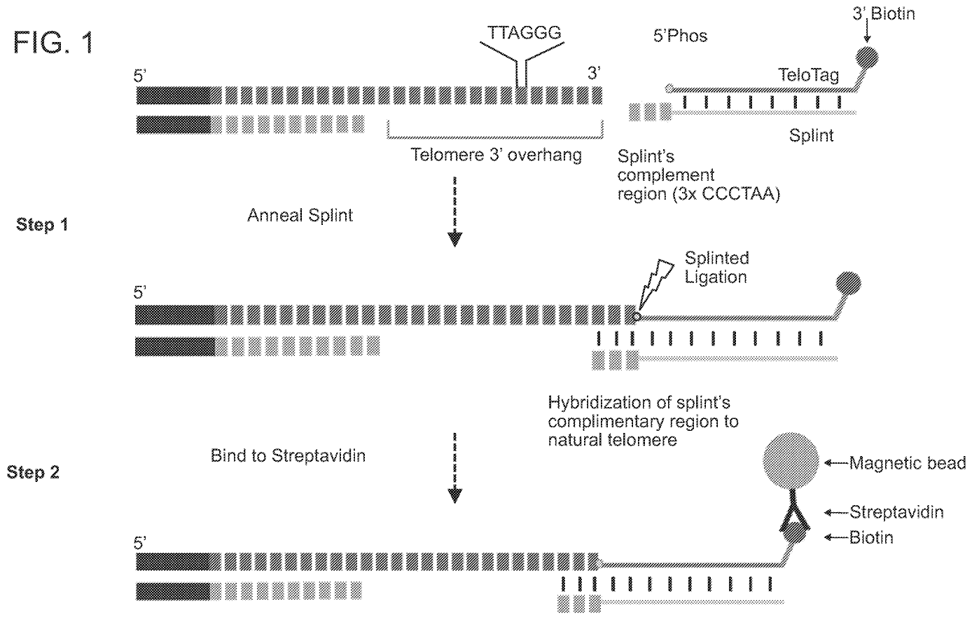

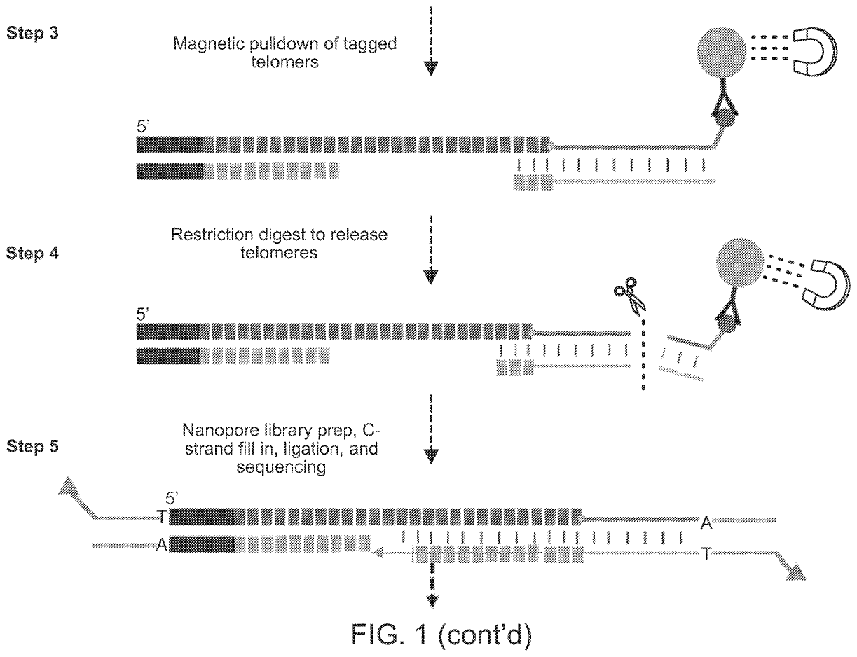

Provided herein are methods for determining a telomere length of a DNA molecule from a biological sample that include (a) providing a telomere tagging probe comprising a biotin adapter; (b) providing a splint oligonucleotide, wherein the splint oligonucleotide specifically binds to at least a portion of a telomere on the DNA molecule and to at least a portion of the telomere tagging probe; (c) attaching the telomere tagging probe to the telomere to generate a tagged telomere sequence; (d) contacting the tagged telomere sequence with a streptavidin-functionalized bead, wherein the biotin adapter of the tagged telomere sequence binds the streptavidin-functionalized bead; (e) recovering the tagged telomere sequence; and (f) analyzing the tagged telomere sequence, thereby determining the telomere length of the DNA molecule (FIG. 1).

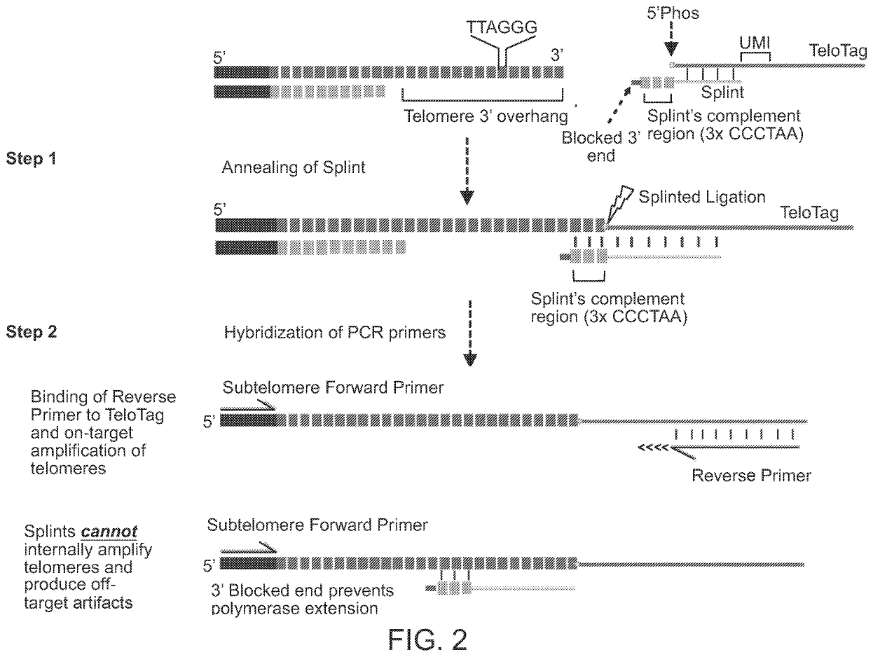

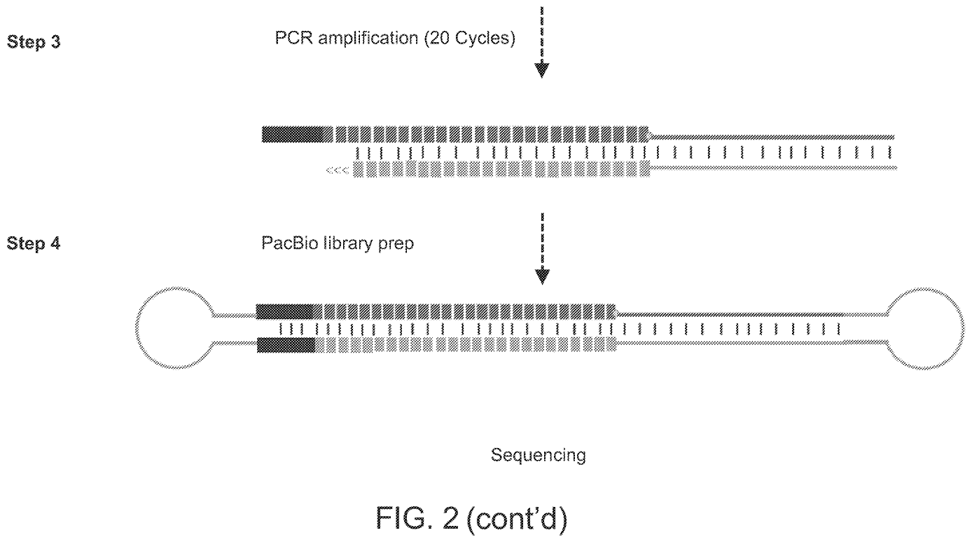

Also provided herein are methods for determining a telomere length of a DNA molecule from a biological sample that include (a) providing a telomere tagging probe, wherein the telomere tagging probe comprises a unique molecular identifier (UMI) sequence; (b) providing a splint oligonucleotide, wherein the splint oligonucleotide specifically binds to at least a portion of a telomere on the DNA molecule and to at least a portion of the telomere tagging probe; (c) attaching the telomere tagging probe to the telomere to generate a tagged telomere sequence; (d) contacting the tagged telomere sequence with a forward primer and a reverse primer, wherein the forward primer binds to a subtelomere of the telomere, and the reverse primer binds to at least a portion of the telomere tagging probe; (e) amplifying the tagged telomere sequence to generate an amplified tagged telomere sequence; and (f) analyzing the amplified tagged telomere sequence, thereby determining the telomere length of the DNA molecule (FIG. 2).

In some embodiments, the DNA molecule to be analyzed is present in and/or can be obtained from a biological sample. The biological sample may be obtained from a subject. In some embodiments, the subject is a mammal. Examples of mammals from which the DNA molecule can be obtained and used as a nucleic acid template in the methods described herein include, without limitation, humans, non-human primates (e.g., monkeys), dogs, cats, sheep, rabbits, mice, hamsters, and rats. In some embodiments, the subject is a human subject. In some embodiments, the biological sample is blood or a blood fraction from the subject. In some embodiments, the biological sample is sera from the subject. In some embodiments, the biological sample is a blood sample. In some embodiments, the biological sample is a tissue sample.

### Direct Telomere Profiling

### Telomere Tagging Probe

As used herein, a “probe” and “tagging probe” can refer to any molecule capable of capturing (directly or indirectly) and/or labelling an analyte (e.g., DNA molecule) in a biological sample. In some embodiments of direct telomere profiling, the telomere tagging probe is a nucleic acid. In some embodiments of direct telomere profiling, the telomere tagging probe is a conjugate (e.g., an oligonucleotide-biotin adapter conjugate). In some embodiments of direct telomere profiling, a telomere tagging probe can be coupled to a nucleic acid sequence using any one of many different techniques including, but not limited to, ligation, hybridization, and tagmentation. In some embodiments of direct telomere profiling, a tagging probe can include nucleic acid sequences that add a function, e.g., spacer sequences, primer sequences/sites, and/or sample barcode sequences. In some embodiments, a telomere tagging probe can include a plurality of nucleic acid sequences, wherein the plurality of nucleic acid sequences can be attached to each other via ligation of the nucleic acids.

In some embodiments of direct telomere profiling, methods described herein include attaching a telomere tagging probe to a 3′ end of the telomere to generate a tagged telomere sequence. In some embodiments, the attaching step comprises ligating the telomere tagging probe to the 3′ end of the telomere. In some embodiments, the ligating comprises cycling ligation. In some embodiments, the ligating comprises using a DNA ligase enzyme. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For example, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase), and Ampligase®. Derivatives, e.g., sequence-modified derivatives, and/or mutants thereof, can also be used. In some embodiments, the ligating comprises a Taq DNA ligase.

### Splint Oligonucleotide

In some embodiments of direct telomere profiling, methods provided herein include use of a splint oligonucleotide comprising a nucleic acid sequence that specifically binds to at least a portion of the telomere and to at least a portion of the telomere tagging probe. As used herein, a “splint oligonucleotide” is an oligonucleotide that, when hybridized to other polynucleotides, acts to position the polynucleotides next to one another so that they can be attached (e.g., ligated together). In some embodiments, the splint oligonucleotide is DNA or RNA. In some embodiments, the splint oligonucleotide can include a nucleotide sequence that is partially complementary to nucleotide sequences from two or more different oligonucleotides. In general, an RNA ligase, a DNA ligase, or another other variety of ligase is used to ligate two nucleotide sequences together.

In some embodiments, the splint oligonucleotide is between 10 and 50 oligonucleotides in length (e.g., between 10 and 45, between 10 and 40, between 10 and 35, between 10 and 30, between 10 and 25, between 10 and 20, or between 10 and 15, between 15 and 50, between 15 and 45, between 15 and 40, between 15 and 35, between 15 and 30, between 15 and 25, between 15 and 20, between 20 and 50, between 20 and 45, between 20 and 40, between 20 and 35, between 20 and 30, between 20 and 25, between 25 and 50, between 25 and 45, between 25 and 40, between 25 and 35, between 25 and 30, between 30 and 50, between 30 and 45, between 30 and 40, between 30 and 35, between 35 and 50, between 35 and 45, between 35 and 40, between 40 and 50, between 40 and 45, or between 45 and 50 oligonucleotides in length).

In some embodiments, the splint oligonucleotide is not blocked at its 3′ end.

In some embodiments, the splint oligonucleotide comprises SEQ ID NO: 1-143.

| | | | Puri- | SEQ ID |
|---|---|---|---|---|
| Name | Sequence | Scale | fication | NO |
| NB01-Phos | /5Phos/CCCTCCGATACACAAAGACACCGACAACTTTCTTTG | 1 μm | HPLC | 1 |
| | CGTACAGCAATCAGGCGCGCCCGATCGGAATTCTTAATTA | | | |
| | ATGGTGGACTCTATGACCGTTCA/3BioTEG/ | | | |
| NB02-PhosNew | /5Phos/CCCTCCGATAACAGACGACTACAAACGGAATCGAT | 1 μm | HPLC | 2 |
| | GCGTACAGCAATCAGGCGCGCCCGATCGGAATTCTTAATT | | | |
| | AATGGTGGACTCTATGACCGTTCA/3BioTEG/ | | | |
| NB15-Phos | /5Phos/CCCTCCGATAAGGTCTACCTCGCTAACACCACTGTG | 1 μm | HPLC | 3 |
| | CGTACAGCAATCAGGCGCGCCCGATCGGAATTCTTAATTA | | | |
| | ATGGTGGACTCTATGACCGTTCA/3BioTEG/ | | | |
| NB19-Phos | /5Phos/CCCTCCGATAGTTCCTCGTGCAGTGTCAAGAGATTG | 1 μm | HPLC | 4 |
| | CGTACAGCAATCAGGCGCGCCCGATCGGAATTCTTAATTA | | | |
| | ATGGTGGACTCTATGACCGTTCA/3BioTEG/ | | | |
| NB20-Phos | /5Phos/CCCTCCGATATTGCGTCCTGTTACGAGAACTCATTG | 1 μm | HPLC | 5 |
| | CGTACAGCAATCAGGCGCGCCCGATCGGAATTCTTAATTA | | | |
| | ATGGTGGACTCTATGACCGTTCA/3BioTEG/ | | | |
| NB66-Phos | /5Phos/CCCTCCGATACCGATCCTTGTGGCTTCTAACTTCTGC | 1 μm | HPLC | 6 |
| | GTACAGCAATCAGGCGCGCCCGATCGGAATTCTTAATTAA | | | |
| | TGGTGGACTCTATGACCGTTCA/3BioTEG/ | | | |
| NB67-Phos | /5Phos/CCCTCCGATAGTTTGTCATACTCGTGTGCTCACCTGC | 1 μm | HPLC | 7 |
| | GTACAGCAATCAGGCGCGCCCGATCGGAATTCTTAATTAA | | | |
| | TGGTGGACTCTATGACCGTTCA/3BioTEG/ | | | |
| NB69-Phos | /5Phos/CCCTCCGATATACAGTCCGAGCCTCATGTGATCTTG | 1 μm | HPLC | 8 |
| | CGTACAGCAATCAGGCGCGCCCGATCGGAATTCTTAATTA | | | |
| | ATGGTGGACTCTATGACCGTTCA/3BioTEG/ | | | |
| NB70-Phos | /5Phos/CCCTCCGATAACCGAGATCCTACGAATGGAGTGTTG | 1 μm | HPLC | 9 |
| | CGTACAGCAATCAGGCGCGCCCGATCGGAATTCTTAATTA | | | |
| | ATGGTGGACTCTATGACCGTTCA/3BioTEG/ | | | |
| NB72-Phos | /5Phos/CCCTCCGATATAGCTGACTGTCTTCCATACCGACTG | 1 μm | HPLC | 10 |
| | CGTACAGCAATCAGGCGCGCCCGATCGGAATTCTTAATTA | | | |
| | ATGGTGGACTCTATGACCGTTCA/3BioTEG/ | | | |
| NB40_PhosBiotin_ | /5Phos/CCCTCCGATATAGTTTGGATGACCAAGGATAGCCGA | 1 μm | HPLC | 11 |
| Indi_EcoRI_PvuI_ | ATTCGATCGTTAATTAATAGGGATAACAGGGTAATGGCGC | | | |
| PacI_IsceI_AscI | GCCATGGTGGACTCTATGACCAT/3BioTEG/ | | | |
| NB40_Splint1 | CATGGCGCGCCATTACCCTGTTATCCCTATTAATTAACGAT | 250 nm | HPLC | 12 |
| | CGAATTCGGCTATCCTTGGTCATCCAAACTATATCGGAGG | | | |
| | GCCCTAACCCTAACCCTAA | | | |
| NB40_Splint2 | CATGGCGCGCCATTACCCTGTTATCCCTATTAATTAACGAT | 250 nm | HPLC | 13 |
| | CGAATTCGGCTATCCTTGGTCATCCAAACTATATCGGAGG | | | |
| | GCCTAACCCTAACCCTAAC | | | |
| NB40_Splint3 | CATGGCGCGCCATTACCCTGTTATCCCTATTAATTAACGAT | 250 nm | HPLC | 14 |
| | CGAATTCGGCTATCCTTGGTCATCCAAACTATATCGGAGG | | | |
| | GCTAACCCTAACCCTAACC | | | |
| NB40_Splint4 | CATGGCGCGCCATTACCCTGTTATCCCTATTAATTAACGAT | 250 nm | HPLC | 15 |
| | CGAATTCGGCTATCCTTGGTCATCCAAACTATATCGGAGG | | | |
| | GTAACCCTAACCCTAACCC | | | |
| NB40_Splint5 | CATGGCGCGCCATTACCCTGTTATCCCTATTAATTAACGAT | 250 nm | HPLC | 16 |
| | CGAATTCGGCTATCCTTGGTCATCCAAACTATATCGGAGG | | | |
| | GAACCCTAACCCTAACCCT | | | |
| NB40_Splint6 | CATGGCGCGCCATTACCCTGTTATCCCTATTAATTAACGAT | 250 nm | HPLC | 17 |
| | CGAATTCGGCTATCCTTGGTCATCCAAACTATATCGGAGG | | | |
| | GACCCTAACCCTAACCCTA | | | |
| NB43_PhosBiotin_ | /5Phos/CCCTCCGATACTTTCGTTGTTGACTCGACGGTAGGA | 1 μm | HPLC | 18 |
| IsceI | ATTCGATCGTTAATTAATAGGGATAACAGGGTAATGGCGC | | | |
| | GCCATGGTGGACTCTATGACCAT/3BioTEG/ | | | |
| NB43_Splint1 | CATGGCGCGCCATTACCCTGTTATCCCTATTAATTAACGAT | 250 nm | HPLC | 19 |
| | CGAATTCCTACCGTCGAGTCAACAACGAAAGTATCGGAGG | | | |
| | GCCCTAACCCTAACCCTAA | | | |
| NB43_Splint2 | CATGGCGCGCCATTACCCTGTTATCCCTATTAATTAACGAT | 250 nm | HPLC | 20 |
| | CGAATTCCTACCGTCGAGTCAACAACGAAAGTATCGGAGG | | | |
| | GCCTAACCCTAACCCTAAC | | | |
| NB43_Splint3 | CATGGCGCGCCATTACCCTGTTATCCCTATTAATTAACGAT | 250 nm | HPLC | 21 |
| | CGAATTCCTACCGTCGAGTCAACAACGAAAGTATCGGAGG | | | |
| | GCTAACCCTAACCCTAACC | | | |
| NB43_Splint4 | CATGGCGCGCCATTACCCTGTTATCCCTATTAATTAACGAT | 250 nm | HPLC | 22 |
| | CGAATTCCTACCGTCGAGTCAACAACGAAAGTATCGGAGG | | | |
| | GTAACCCTAACCCTAACCC | | | |
| NB43_Splint5 | CATGGCGCGCCATTACCCTGTTATCCCTATTAATTAACGAT | 250 nm | HPLC | 23 |
| | CGAATTCCTACCGTCGAGTCAACAACGAAAGTATCGGAGG | | | |
| | GAACCCTAACCCTAACCCT | | | |
| NB43_Splint6 | CATGGCGCGCCATTACCCTGTTATCCCTATTAATTAACGAT | 250 nm | HPLC | 24 |
| | CGAATTCCTACCGTCGAGTCAACAACGAAAGTATCGGAGG | | | |
| | GACCCTAACCCTAACCCTA | | | |
| NB50_Indi_ | /5Phos/CCCTCCGATAATGGACTTTGGTAACTTCCTGCGTTG | 1 μm | HPLC | 25 |
| AscIEcoPvIPacI | CGTACAGCAATCAGGCGCGCCGAATTCCGATCGTTAATTA | | | |
| | ATGGTGGACTCTATGACCGTTCA/3BioTEG/ | | | |
| NB50_Splint1 | CACCATTAATTAACGATCGGAATTCGGCGCGCCTGATTGC | 250 nm | HPLC | 26 |
| | TGTACGCAACGCAGGAAGTTACCAAAGTCCATTATCGGAG | | | |
| | GGCCCTAACCCTAACCCTAA | | | |
| NB50_Splint2 | CACCATTAATTAACGATCGGAATTCGGCGCGCCTGATTGC | 250 nm | HPLC | 27 |
| | TGTACGCAACGCAGGAAGTTACCAAAGTCCATTATCGGAG | | | |
| | GGCCTAACCCTAACCCTAAC | | | |
| NB50_Splint3 | CACCATTAATTAACGATCGGAATTCGGCGCGCCTGATTGC | 250 nm | HPLC | 28 |
| | TGTACGCAACGCAGGAAGTTACCAAAGTCCATTATCGGAG | | | |
| | GGCTAACCCTAACCCTAACC | | | |
| NB50_Splint4 | CACCATTAATTAACGATCGGAATTCGGCGCGCCTGATTGC | 250 nm | HPLC | 29 |
| | TGTACGCAACGCAGGAAGTTACCAAAGTCCATTATCGGAG | | | |
| | GGTAACCCTAACCCTAACCC | | | |
| NB50_Splint5 | CACCATTAATTAACGATCGGAATTCGGCGCGCCTGATTGC | 250 nm | HPLC | 30 |
| | TGTACGCAACGCAGGAAGTTACCAAAGTCCATTATCGGAG | | | |
| | GGAACCCTAACCCTAACCCT | | | |
| NB50_Splint6 | CACCATTAATTAACGATCGGAATTCGGCGCGCCTGATTGC | 250 nm | HPLC | 31 |
| | TGTACGCAACGCAGGAAGTTACCAAAGTCCATTATCGGAG | | | |
| | GGACCCTAACCCTAACCCTA | | | |
| NB12_ | AGAGTCCACCATGAATTCCACTGACAATGTAACTGATTGC | 250 nm | HPLC | 32 |
| correctedSplint1 | TGTACGCACAGGTAGAAAGAAGCAGAATCGGATATCGGA | | | |
| | GGGCCCTAACCCTAACCCTAA | | | |
| NB12_ | AGAGTCCACCATGAATTCCACTGACAATGTAACTGATTGC | 250 nm | HPLC | 33 |
| correctedSplint2 | TGTACGCACAGGTAGAAAGAAGCAGAATCGGATATCGGA | | | |
| | GGGCCTAACCCTAACCCTAAC | | | |
| NB12_ | AGAGTCCACCATGAATTCCACTGACAATGTAACTGATTGC | 250 nm | HPLC | 34 |
| correctedSplint3 | TGTACGCACAGGTAGAAAGAAGCAGAATCGGATATCGGA | | | |
| | GGGCTAACCCTAACCCTAACC | | | |
| NB12_ | AGAGTCCACCATGAATTCCACTGACAATGTAACTGATTGC | 250 nm | HPLC | 35 |
| correctedSplint4 | TGTACGCACAGGTAGAAAGAAGCAGAATCGGATATCGGA | | | |
| | GGGTAACCCTAACCCTAACCC | | | |
| NB12_ | AGAGTCCACCATGAATTCCACTGACAATGTAACTGATTGC | 250 nm | HPLC | 36 |
| correctedSplint5 | TGTACGCACAGGTAGAAAGAAGCAGAATCGGATATCGGA | | | |
| | GGGAACCCTAACCCTAACCCT | | | |
| NB12_ | AGAGTCCACCATGAATTCCACTGACAATGTAACTGATTGC | 250 nm | HPLC | 37 |
| correctedSplint6 | TGTACGCACAGGTAGAAAGAAGCAGAATCGGATATCGGA | | | |
| | GGGACCCTAACCCTAACCCTA | | | |
| NB65_indi_AscI_ | /5Phos/CCCTCCGATATTCTCAGTCTTCCTCCAGACAAGGTG | 1 μm | HPLC | 38 |
| EcoRI_PvuI- | CGTACAGCAATCAGGCGCGCCGAATTCCGATCGTTAATTA | | | |
| IIF_PacI | ATGGTGGACTCTATGACCGTTCA/3BioTEG/ | | | |
| NB65_splint1 | CACCATTAATTAACGATCGGAATTCGGCGCGCCTGATTGC | 250 nm | HPLC | 39 |
| | TGTACGCACCTTGTCTGGAGGAAGACTGAGAATATCGGAG | | | |
| | GGCCCTAACCCTAACCCTAA | | | |
| NB65_splint2 | CACCATTAATTAACGATCGGAATTCGGCGCGCCTGATTGC | 250 nm | HPLC | 40 |
| | TGTACGCACCTTGTCTGGAGGAAGACTGAGAATATCGGAG | | | |
| | GGCCTAACCCTAACCCTAAC | | | |
| NB65_splint3 | CACCATTAATTAACGATCGGAATTCGGCGCGCCTGATTGC | 250 nm | HPLC | 41 |
| | TGTACGCACCTTGTCTGGAGGAAGACTGAGAATATCGGAG | | | |
| | GGCTAACCCTAACCCTAACC | | | |
| NB65_splint4 | CACCATTAATTAACGATCGGAATTCGGCGCGCCTGATTGC | 250 nm | HPLC | 42 |
| | TGTACGCACCTTGTCTGGAGGAAGACTGAGAATATCGGAG | | | |
| | GGTAACCCTAACCCTAACCC | | | |
| NB65_splint5 | CACCATTAATTAACGATCGGAATTCGGCGCGCCTGATTGC | 250 nm | HPLC | 43 |
| | TGTACGCACCTTGTCTGGAGGAAGACTGAGAATATCGGAG | | | |
| | GGAACCCTAACCCTAACCCT | | | |
| NB65_splint6 | CACCATTAATTAACGATCGGAATTCGGCGCGCCTGATTGC | 250 nm | HPLC | 44 |
| | TGTACGCACCTTGTCTGGAGGAAGACTGAGAATATCGGAG | | | |
| | GGACCCTAACCCTAACCCTA | | | |
| NB68_indi_AscI_ | /5Phos/CCCTCCGATAGAATCTAAGCAAACACGAAGGTGGT | 1 μm | HPLC | 45 |
| EcoRI_PvuI- | GCGTACAGCAATCAGGCGCGCCGAATTCCGATCGTTAATT | | | |
| HF_PacI | AATGGTGGACTCTATGACCGTTCA/3BioTEG/ | | | |
| NB68_splint1 | CACCATTAATTAACGATCGGAATTCGGCGCGCCTGATTGC | 250 nm | HPLC | 46 |
| | TGTACGCACCACCTTCGTGTTTGCTTAGATTCTATCGGAGG | | | |
| | GCCCTAACCCTAACCCTAA | | | |
| NB68_splint2 | CACCATTAATTAACGATCGGAATTCGGCGCGCCTGATTGC | 250 nm | HPLC | 47 |
| | TGTACGCACCACCTTCGTGTTTGCTTAGATTCTATCGGAGG | | | |
| | GCCTAACCCTAACCCTAAC | | | |
| NB68_splint3 | CACCATTAATTAACGATCGGAATTCGGCGCGCCTGATTGC | 250 nm | HPLC | 48 |
| | TGTACGCACCACCTTCGTGTTTGCTTAGATTCTATCGGAGG | | | |
| | GCTAACCCTAACCCTAACC | | | |
| NB68_splint4 | CACCATTAATTAACGATCGGAATTCGGCGCGCCTGATTGC | 250 nm | HPLC | 49 |
| | TGTACGCACCACCTTCGTGTTTGCTTAGATTCTATCGGAGG | | | |
| | GTAACCCTAACCCTAACCC | | | |
| NB68_splint5 | CACCATTAATTAACGATCGGAATTCGGCGCGCCTGATTGC | 250 nm | HPLC | 50 |
| | TGTACGCACCACCTTCGTGTTTGCTTAGATTCTATCGGAGG | | | |
| | GAACCCTAACCCTAACCCT | | | |
| NB68_splint6 | CACCATTAATTAACGATCGGAATTCGGCGCGCCTGATTGC | 250 nm | HPLC | 51 |
| | TGTACGCACCACCTTCGTGTTTGCTTAGATTCTATCGGAGG | | | |
| | GACCCTAACCCTAACCCTA | | | |
| NB88_indi_AscI_ | /5Phos/CCCTCCGATATCTTCTACTACCGATCCGAAGCAGTG | 1 μm | HPLC | 52 |
| EcoRI_PvuI- | CGTACAGCAATCAGGCGCGCCGAATTCCGATCGTTAATTA | | | |
| HF_PacI | ATGGTGGACTCTATGACCGTTCA/3BioTEG/ | | | |
| NB88_splint1 | CACCATTAATTAACGATCGGAATTCGGCGCGCCTGATTGC | 250 nm | HPLC | 53 |
| | TGTACGCACTGCTTCGGATCGGTAGTAGAAGATATCGGAG | | | |
| | GGCCCTAACCCTAACCCTAA | | | |
| NB88_splint2 | CACCATTAATTAACGATCGGAATTCGGCGCGCCTGATTGC | 250 nm | HPLC | 54 |
| | TGTACGCACTGCTTCGGATCGGTAGTAGAAGATATCGGAG | | | |
| | GGCCTAACCCTAACCCTAAC | | | |
| NB88_splint3 | CACCATTAATTAACGATCGGAATTCGGCGCGCCTGATTGC | 250 nm | HPLC | 55 |
| | TGTACGCACTGCTTCGGATCGGTAGTAGAAGATATCGGAG | | | |
| | GGCTAACCCTAACCCTAACC | | | |
| NB88_splint4 | CACCATTAATTAACGATCGGAATTCGGCGCGCCTGATTGC | 250 nm | HPLC | 56 |
| | TGTACGCACTGCTTCGGATCGGTAGTAGAAGATATCGGAG | | | |
| | GGTAACCCTAACCCTAACCC | | | |
| NB88_splint5 | CACCATTAATTAACGATCGGAATTCGGCGCGCCTGATTGC | 250 nm | HPLC | 57 |
| | TGTACGCACTGCTTCGGATCGGTAGTAGAAGATATCGGAG | | | |
| | GGAACCCTAACCCTAACCCT | | | |
| NB88_splint6 | CACCATTAATTAACGATCGGAATTCGGCGCGCCTGATTGC | 250 nm | HPLC | 58 |
| | TGTACGCACTGCTTCGGATCGGTAGTAGAAGATATCGGAG | | | |
| | GGACCCTAACCCTAACCCTA | | | |
| Iscel Middle cut | /5phos/CCCTCCGATAACGGTATGTCGAGTTCCAGGACTAGA | 1 μm | HPLC | 59 |
| Adapter | ATTCTAGGGATAACAGGGTAATGATCGTTAATTAAGGCGC | | | |
| | GCCATGGTGGACTCTATGACCAT/3bioteg/ | | | |
| NB82_splint1 | CATGGCGCGCCTTAATTAACGATCATTACCCTGTTATCCCT | 250 nm | HPLC | 60 |
| | AGAATTCTAGTCCTGGAACTCGACATACCGTTATCGGAGG | | | |
| | GCCCTAACCCTAACCCTAA | | | |
| NB82_splint2 | CATGGCGCGCCTTAATTAACGATCATTACCCTGTTATCCCT | 250 nm | HPLC | 61 |
| | AGAATTCTAGTCCTGGAACTCGACATACCGTTATCGGAGG | | | |
| | GCCTAACCCTAACCCTAAC | | | |
| NB82_splint3 | CATGGCGCGCCTTAATTAACGATCATTACCCTGTTATCCCT | 250 nm | HPLC | 62 |
| | AGAATTCTAGTCCTGGAACTCGACATACCGTTATCGGAGG | | | |
| | GCTAACCCTAACCCTAACC | | | |
| NB82_splint4 | CATGGCGCGCCTTAATTAACGATCATTACCCTGTTATCCCT | 250 nm | HPLC | 63 |
| | AGAATTCTAGTCCTGGAACTCGACATACCGTTATCGGAGG | | | |
| | GTAACCCTAACCCTAACCC | | | |
| NB82_splint5 | CATGGCGCGCCTTAATTAACGATCATTACCCTGTTATCCCT | 250 nm | HPLC | 64 |
| | AGAATTCTAGTCCTGGAACTCGACATACCGTTATCGGAGG | | | |
| | GAACCCTAACCCTAACCCT | | | |
| NB82_splint6 | CATGGCGCGCCTTAATTAACGATCATTACCCTGTTATCCCT | 250 nm | HPLC | 65 |
| | AGAATTCTAGTCCTGGAACTCGACATACCGTTATCGGAGG | | | |
| | GACCCTAACCCTAACCCTA | | | |
| NB01_splint1 | CACCATTAATTAAGAATTCCGATCGGGCGCGCCTGATTGC | 250 nm | HPLC | 66 |
| | TGTACGCAAAGAAAGTTGTCGGTGTCTTTGTGTATCGGAG | | | |
| | GGCCCTAACCCTAACCCTAA | | | |
| NB01_splint2 | CACCATTAATTAAGAATTCCGATCGGGCGCGCCTGATTGC | 250 nm | HPLC | 67 |
| | TGTACGCAAAGAAAGTTGTCGGTGTCTTTGTGTATCGGAG | | | |
| | GGCCTAACCCTAACCCTAAC | | | |
| NB01_splint3 | CACCATTAATTAAGAATTCCGATCGGGCGCGCCTGATTGC | 250 nm | HPLC | 68 |
| | TGTACGCAAAGAAAGTTGTCGGTGTCTTTGTGTATCGGAG | | | |
| | GGCTAACCCTAACCCTAACC | | | |
| NB01_splint4 | CACCATTAATTAAGAATTCCGATCGGGCGCGCCTGATTGC | 250 nm | HPLC | 69 |
| | TGTACGCAAAGAAAGTTGTCGGTGTCTTTGTGTATCGGAG | | | |
| | GGTAACCCTAACCCTAACCC | | | |
| NB01_splint5 | CACCATTAATTAAGAATTCCGATCGGGCGCGCCTGATTGC | 250 nm | HPLC | 70 |
| | TGTACGCAAAGAAAGTTGTCGGTGTCTTTGTGTATCGGAG | | | |
| | GGAACCCTAACCCTAACCCT | | | |
| NB01_splint6 | CACCATTAATTAAGAATTCCGATCGGGCGCGCCTGATTGC | 250 nm | HPLC | 71 |
| | TGTACGCAAAGAAAGTTGTCGGTGTCTTTGTGTATCGGAG | | | |
| | GGACCCTAACCCTAACCCTA | | | |
| NB02_splint1 | CACCATTAATTAAGAATTCCGATCGGGCGCGCCTGATTGC | 250 nm | HPLC | 72 |
| | TGTACGCATCGATTCCGTTTGTAGTCGTCTGTTATCGGAGG | | | |
| | GCCCTAACCCTAACCCTAA | | | |
| NB02_splint2 | CACCATTAATTAAGAATTCCGATCGGGCGCGCCTGATTGC | 250 nm | HPLC | 73 |
| | TGTACGCATCGATTCCGTTTGTAGTCGTCTGTTATCGGAGG | | | |
| | GCCTAACCCTAACCCTAAC | | | |
| NB02_splint3 | CACCATTAATTAAGAATTCCGATCGGGCGCGCCTGATTGC | 250 nm | HPLC | 74 |
| | TGTACGCATCGATTCCGTTTGTAGTCGTCTGTTATCGGAGG | | | |
| | GCTAACCCTAACCCTAACC | | | |
| NB02_splint4 | CACCATTAATTAAGAATTCCGATCGGGCGCGCCTGATTGC | 250 nm | HPLC | 75 |
| | TGTACGCATCGATTCCGTTTGTAGTCGTCTGTTATCGGAGG | | | |
| | GTAACCCTAACCCTAACCC | | | |
| NB02_splint5 | CACCATTAATTAAGAATTCCGATCGGGCGCGCCTGATTGC | 250 nm | HPLC | 76 |
| | TGTACGCATCGATTCCGTTTGTAGTCGTCTGTTATCGGAGG | | | |
| | GAACCCTAACCCTAACCCT | | | |
| NB02_splint6 | CACCATTAATTAAGAATTCCGATCGGGCGCGCCTGATTGC | 250 nm | HPLC | 77 |
| | TGTACGCATCGATTCCGTTTGTAGTCGTCTGTTATCGGAGG | | | |
| | GACCCTAACCCTAACCCTA | | | |
| NB15_splint1 | CACCATTAATTAAGAATTCCGATCGGGCGCGCCTGATTGC | 250 nm | HPLC | 78 |
| | TGTACGCACAGTGGTGTTAGCGAGGTAGACCTTATCGGAG | | | |
| | GGCCCTAACCCTAACCCTAA | | | |
| NB15_splint2 | CACCATTAATTAAGAATTCCGATCGGGCGCGCCTGATTGC | 250 nm | HPLC | 79 |
| | TGTACGCACAGTGGTGTTAGCGAGGTAGACCTTATCGGAG | | | |
| | GGCCTAACCCTAACCCTAAC | | | |
| NB15_splint3 | CACCATTAATTAAGAATTCCGATCGGGCGCGCCTGATTGC | 250 nm | HPLC | 80 |
| | TGTACGCACAGTGGTGTTAGCGAGGTAGACCTTATCGGAG | | | |
| | GGCTAACCCTAACCCTAACC | | | |
| NB15_splint4 | CACCATTAATTAAGAATTCCGATCGGGCGCGCCTGATTGC | 250 nm | HPLC | 81 |
| | TGTACGCACAGTGGTGTTAGCGAGGTAGACCTTATCGGAG | | | |
| | GGTAACCCTAACCCTAACCC | | | |
| NB15_splint5 | CACCATTAATTAAGAATTCCGATCGGGCGCGCCTGATTGC | 250 nm | HPLC | 82 |
| | TGTACGCACAGTGGTGTTAGCGAGGTAGACCTTATCGGAG | | | |
| | GGAACCCTAACCCTAACCCT | | | |
| NB15_splint6 | CACCATTAATTAAGAATTCCGATCGGGCGCGCCTGATTGC | 250 nm | HPLC | 83 |
| | TGTACGCACAGTGGTGTTAGCGAGGTAGACCTTATCGGAG | | | |
| | GGACCCTAACCCTAACCCTA | | | |
| NB19_splint1 | CACCATTAATTAAGAATTCCGATCGGGCGCGCCTGATTGC | 250 nm | HPLC | 84 |
| | TGTACGCAATCTCTTGACACTGCACGAGGAACTATCGGAG | | | |
| | GGCCCTAACCCTAACCCTAA | | | |
| NB19_splint2 | CACCATTAATTAAGAATTCCGATCGGGCGCGCCTGATTGC | 250 nm | HPLC | 85 |
| | TGTACGCAATCTCTTGACACTGCACGAGGAACTATCGGAG | | | |
| | GGCCTAACCCTAACCCTAAC | | | |
| NB19_splint3 | CACCATTAATTAAGAATTCCGATCGGGCGCGCCTGATTGC | 250 nm | HPLC | 86 |
| | TGTACGCAATCTCTTGACACTGCACGAGGAACTATCGGAG | | | |
| | GGCTAACCCTAACCCTAACC | | | |
| NB19_splint4 | CACCATTAATTAAGAATTCCGATCGGGCGCGCCTGATTGC | 250 nm | HPLC | 87 |
| | TGTACGCAATCTCTTGACACTGCACGAGGAACTATCGGAG | | | |
| | GGTAACCCTAACCCTAACCC | | | |
| NB19_splint5 | CACCATTAATTAAGAATTCCGATCGGGCGCGCCTGATTGC | 250 nm | HPLC | 88 |
| | TGTACGCAATCTCTTGACACTGCACGAGGAACTATCGGAG | | | |
| | GGAACCCTAACCCTAACCCT | | | |
| NB19_splint6 | CACCATTAATTAAGAATTCCGATCGGGCGCGCCTGATTGC | 250 nm | HPLC | 89 |
| | TGTACGCAATCTCTTGACACTGCACGAGGAACTATCGGAG | | | |
| | GGACCCTAACCCTAACCCTA | | | |
| NB20_splint1 | CACCATTAATTAAGAATTCCGATCGGGCGCGCCTGATTGC | 250 nm | HPLC | 90 |
| | TGTACGCAATGAGTTCTCGTAACAGGACGCAATATCGGAG | | | |
| | GGCCCTAACCCTAACCCTAA | | | |
| NB20_splint2 | CACCATTAATTAAGAATTCCGATCGGGCGCGCCTGATTGC | 250 nm | HPLC | 91 |
| | TGTACGCAATGAGTTCTCGTAACAGGACGCAATATCGGAG | | | |
| | GGCCTAACCCTAACCCTAAC | | | |
| NB20_splint3 | CACCATTAATTAAGAATTCCGATCGGGCGCGCCTGATTGC | 250 nm | HPLC | 92 |
| | TGTACGCAATGAGTTCTCGTAACAGGACGCAATATCGGAG | | | |
| | GGCTAACCCTAACCCTAACC | | | |
| NB20_splint4 | CACCATTAATTAAGAATTCCGATCGGGCGCGCCTGATTGC | 250 nm | HPLC | 93 |
| | TGTACGCAATGAGTTCTCGTAACAGGACGCAATATCGGAG | | | |
| | GGTAACCCTAACCCTAACCC | | | |
| NB20_splint5 | CACCATTAATTAAGAATTCCGATCGGGCGCGCCTGATTGC | 250 nm | HPLC | 94 |
| | TGTACGCAATGAGTTCTCGTAACAGGACGCAATATCGGAG | | | |
| | GGAACCCTAACCCTAACCCT | | | |
| NB20_splint6 | CACCATTAATTAAGAATTCCGATCGGGCGCGCCTGATTGC | 250 nm | HPLC | 95 |
| | TGTACGCAATGAGTTCTCGTAACAGGACGCAATATCGGAG | | | |
| | GGACCCTAACCCTAACCCTA | | | |

NB66

| | | | | |
|---|---|---|---|---|
| NB66_splint1 | CACCATTAATTAAGAATTCCGATCGGGCGCGCCTGATTGC | 250 nm | HPLC | 96 |
| | TGTACGCAGAAGTTAGAAGCCACAAGGATCGGTATCGGA | | | |
| | GGGCCCTAACCCTAACCCTAA | | | |
| NB66_splint2 | CACCATTAATTAAGAATTCCGATCGGGCGCGCCTGATTGC | 250 nm | HPLC | 97 |
| | TGTACGCAGAAGTTAGAAGCCACAAGGATCGGTATCGGA | | | |
| | GGGCCTAACCCTAACCCTAAC | | | |
| NB66_splint3 | CACCATTAATTAAGAATTCCGATCGGGCGCGCCTGATTGC | 250 nm | HPLC | 98 |
| | TGTACGCAGAAGTTAGAAGCCACAAGGATCGGTATCGGA | | | |
| | GGGCTAACCCTAACCCTAACC | | | |
| NB66_splint4 | CACCATTAATTAAGAATTCCGATCGGGCGCGCCTGATTGC | 250 nm | HPLC | 99 |
| | TGTACGCAGAAGTTAGAAGCCACAAGGATCGGTATCGGA | | | |
| | GGGTAACCCTAACCCTAACCC | | | |
| NB66_splint5 | CACCATTAATTAAGAATTCCGATCGGGCGCGCCTGATTGC | 250 nm | HPLC | 100 |
| | TGTACGCAGAAGTTAGAAGCCACAAGGATCGGTATCGGA | | | |
| | GGGAACCCTAACCCTAACCCT | | | |
| NB66_splint6 | CACCATTAATTAAGAATTCCGATCGGGCGCGCCTGATTGC | 250 nm | HPLC | 101 |
| | TGTACGCAGAAGTTAGAAGCCACAAGGATCGGTATCGGA | | | |
| | GGGACCCTAACCCTAACCCTA | | | |
| NB67_splint1 | CACCATTAATTAAGAATTCCGATCGGGCGCGCCTGATTGC | 250 nm | HPLC | 102 |
| | TGTACGCAGGTGAGCACACGAGTATGACAAACTATCGGAG | | | |
| | GGCCCTAACCCTAACCCTAA | | | |
| NB67_splint2 | CACCATTAATTAAGAATTCCGATCGGGCGCGCCTGATTGC | 250 nm | HPLC | 103 |
| | TGTACGCAGGTGAGCACACGAGTATGACAAACTATCGGAG | | | |
| | GGCCTAACCCTAACCCTAAC | | | |
| NB67_splint3 | CACCATTAATTAAGAATTCCGATCGGGCGCGCCTGATTGC | 250 nm | HPLC | 104 |
| | TGTACGCAGGTGAGCACACGAGTATGACAAACTATCGGAG | | | |
| | GGCTAACCCTAACCCTAACC | | | |
| NB67_splint4 | CACCATTAATTAAGAATTCCGATCGGGCGCGCCTGATTGC | 250 nm | HPLC | 105 |
| | TGTACGCAGGTGAGCACACGAGTATGACAAACTATCGGAG | | | |
| | GGTAACCCTAACCCTAACCC | | | |
| NB67_splint5 | CACCATTAATTAAGAATTCCGATCGGGCGCGCCTGATTGC | 250 nm | HPLC | 106 |
| | TGTACGCAGGTGAGCACACGAGTATGACAAACTATCGGAG | | | |
| | GGAACCCTAACCCTAACCCT | | | |
| NB67_splint6 | CACCATTAATTAAGAATTCCGATCGGGCGCGCCTGATTGC | 250 nm | HPLC | 107 |
| | TGTACGCAGGTGAGCACACGAGTATGACAAACTATCGGAG | | | |
| | GGACCCTAACCCTAACCCTA | | | |
| NB69_splint1 | CACCATTAATTAAGAATTCCGATCGGGCGCGCCTGATTGC | 250 nm | HPLC | 108 |
| | TGTACGCAAGATCACATGAGGCTCGGACTGTATATCGGAG | | | |
| | GGCCCTAACCCTAACCCTAA | | | |
| NB69_splint2 | CACCATTAATTAAGAATTCCGATCGGGCGCGCCTGATTGC | 250 nm | HPLC | 109 |
| | TGTACGCAAGATCACATGAGGCTCGGACTGTATATCGGAG | | | |
| | GGCCTAACCCTAACCCTAAC | | | |
| NB69_splint3 | CACCATTAATTAAGAATTCCGATCGGGCGCGCCTGATTGC | 250 nm | HPLC | 110 |
| | TGTACGCAAGATCACATGAGGCTCGGACTGTATATCGGAG | | | |
| | GGCTAACCCTAACCCTAACC | | | |
| NB69_splint4 | CACCATTAATTAAGAATTCCGATCGGGCGCGCCTGATTGC | 250 nm | HPLC | 111 |
| | TGTACGCAAGATCACATGAGGCTCGGACTGTATATCGGAG | | | |
| | GGTAACCCTAACCCTAACCC | | | |
| NB69_splint5 | CACCATTAATTAAGAATTCCGATCGGGCGCGCCTGATTGC | 250 nm | HPLC | 112 |
| | TGTACGCAAGATCACATGAGGCTCGGACTGTATATCGGAG | | | |
| | GGAACCCTAACCCTAACCCT | | | |
| NB69_splint6 | CACCATTAATTAAGAATTCCGATCGGGCGCGCCTGATTGC | 250 nm | HPLC | 113 |
| | TGTACGCAAGATCACATGAGGCTCGGACTGTATATCGGAG | | | |
| | GGACCCTAACCCTAACCCTA | | | |
| NB70_splint1 | CACCATTAATTAAGAATTCCGATCGGGCGCGCCTGATTGC | 250 nm | HPLC | 114 |
| | TGTACGCAACACTCCATTCGTAGGATCTCGGTTATCGGAG | | | |
| | GGCCCTAACCCTAACCCTAA | | | |
| NB70_splint2 | CACCATTAATTAAGAATTCCGATCGGGCGCGCCTGATTGC | 250 nm | HPLC | 115 |
| | TGTACGCAACACTCCATTCGTAGGATCTCGGTTATCGGAG | | | |
| | GGCCTAACCCTAACCCTAAC | | | |
| NB70_splint3 | CACCATTAATTAAGAATTCCGATCGGGCGCGCCTGATTGC | 250 nm | HPLC | 116 |
| | TGTACGCAACACTCCATTCGTAGGATCTCGGTTATCGGAG | | | |
| | GGCTAACCCTAACCCTAACC | | | |
| NB70_splint4 | CACCATTAATTAAGAATTCCGATCGGGCGCGCCTGATTGC | 250 nm | HPLC | 117 |
| | TGTACGCAACACTCCATTCGTAGGATCTCGGTTATCGGAG | | | |
| | GGTAACCCTAACCCTAACCC | | | |
| NB70_splint5 | CACCATTAATTAAGAATTCCGATCGGGCGCGCCTGATTGC | 250 nm | HPLC | 118 |
| | TGTACGCAACACTCCATTCGTAGGATCTCGGTTATCGGAG | | | |
| | GGAACCCTAACCCTAACCCT | | | |
| NB70_splint6 | CACCATTAATTAAGAATTCCGATCGGGCGCGCCTGATTGC | 250 nm | HPLC | 119 |
| | TGTACGCAACACTCCATTCGTAGGATCTCGGTTATCGGAG | | | |
| | GGACCCTAACCCTAACCCTA | | | |
| NB72_splint1 | CACCATTAATTAAGAATTCCGATCGGGCGCGCCTGATTGC | 250 nm | HPLC | 120 |
| | TGTACGCAGTCGGTATGGAAGACAGTCAGCTATATCGGAG | | | |
| | GGCCCTAACCCTAACCCTAA | | | |
| NB72_splint2 | CACCATTAATTAAGAATTCCGATCGGGCGCGCCTGATTGC | 250 nm | HPLC | 121 |
| | TGTACGCAGTCGGTATGGAAGACAGTCAGCTATATCGGAG | | | |
| | GGCCTAACCCTAACCCTAAC | | | |
| NB72_splint3 | CACCATTAATTAAGAATTCCGATCGGGCGCGCCTGATTGC | 250 nm | HPLC | 122 |
| | TGTACGCAGTCGGTATGGAAGACAGTCAGCTATATCGGAG | | | |
| | GGCTAACCCTAACCCTAACC | | | |
| NB72_splint4 | CACCATTAATTAAGAATTCCGATCGGGCGCGCCTGATTGC | 250 nm | HPLC | 123 |
| | TGTACGCAGTCGGTATGGAAGACAGTCAGCTATATCGGAG | | | |
| | GGTAACCCTAACCCTAACCC | | | |
| NB72_splint5 | CACCATTAATTAAGAATTCCGATCGGGCGCGCCTGATTGC | 250 nm | HPLC | 124 |
| | TGTACGCAGTCGGTATGGAAGACAGTCAGCTATATCGGAG | | | |
| | GGAACCCTAACCCTAACCCT | | | |
| NB72_splint6 | CACCATTAATTAAGAATTCCGATCGGGCGCGCCTGATTGC | 250 nm | HPLC | 125 |
| | TGTACGCAGTCGGTATGGAAGACAGTCAGCTATATCGGAG | | | |
| | GGACCCTAACCCTAACCCTA | | | |
| NB10_ | GAGTCCACCATGAATTCCACTGACAATGTAACTGATTGCT | 250 nm | HPLC | 126 |
| splint1corrected | GTACGCAAAGCGTTGAAACCTTTGTCCTCTCTATCGGAGG | | | |
| | GCCCTAACCCTAACCCTAA | | | |
| NB10_ | GAGTCCACCATGAATTCCACTGACAATGTAACTGATTGCT | 250 nm | HPLC | 127 |
| splint2corrected | GTACGCAAAGCGTTGAAACCTTTGTCCTCTCTATCGGAGG | | | |
| | GCCTAACCCTAACCCTAAC | | | |
| NB10_ | GAGTCCACCATGAATTCCACTGACAATGTAACTGATTGCT | 250 nm | HPLC | 128 |
| splint3corrected | GTACGCAAAGCGTTGAAACCTTTGTCCTCTCTATCGGAGG | | | |
| | GCTAACCCTAACCCTAACC | | | |
| NB10_ | GAGTCCACCATGAATTCCACTGACAATGTAACTGATTGCT | 250 nm | HPLC | 129 |
| splint4corrected | GTACGCAAAGCGTTGAAACCTTTGTCCTCTCTATCGGAGG | | | |
| | GTAACCCTAACCCTAACCC | | | |
| NB10_ | GAGTCCACCATGAATTCCACTGACAATGTAACTGATTGCT | 250 nm | HPLC | 130 |
| splint5corrected | GTACGCAAAGCGTTGAAACCTTTGTCCTCTCTATCGGAGG | | | |
| | GAACCCTAACCCTAACCCT | | | |
| NB10_ | GAGTCCACCATGAATTCCACTGACAATGTAACTGATTGCT | 250 nm | HPLC | 131 |
| splint6corrected | GTACGCAAAGCGTTGAAACCTTTGTCCTCTCTATCGGAGG | | | |
| | GACCCTAACCCTAACCCTA | | | |
| NB13_corrected_ | GAGTCCACCATGAATTCCACTGACAATGTAACTGATTGCT | 250 nm | HPLC | 132 |
| splint1 | GTACGCATCACACGAGTATGGAAGTCGTTCTTATCGGAGG | | | |
| | GCCCTAACCCTAACCCTAA | | | |
| NB13_corrected_ | GAGTCCACCATGAATTCCACTGACAATGTAACTGATTGCT | 250 nm | HPLC | 133 |
| splint2 | GTACGCATCACACGAGTATGGAAGTCGTTCTTATCGGAGG | | | |
| | GCCTAACCCTAACCCTAAC | | | |
| NB13_corrected_ | GAGTCCACCATGAATTCCACTGACAATGTAACTGATTGCT | 250 nm | HPLC | 134 |
| splint3 | GTACGCATCACACGAGTATGGAAGTCGTTCTTATCGGAGG | | | |
| | GCTAACCCTAACCCTAACC | | | |
| NB13_corrected_ | GAGTCCACCATGAATTCCACTGACAATGTAACTGATTGCT | 250 nm | HPLC | 135 |
| splint4 | GTACGCATCACACGAGTATGGAAGTCGTTCTTATCGGAGG | | | |
| | GTAACCCTAACCCTAACCC | | | |
| NB13_corrected_ | GAGTCCACCATGAATTCCACTGACAATGTAACTGATTGCT | 250 nm | HPLC | 136 |
| splint5 | GTACGCATCACACGAGTATGGAAGTCGTTCTTATCGGAGG | | | |
| | GAACCCTAACCCTAACCCT | | | |
| NB13_corrected_ | GAGTCCACCATGAATTCCACTGACAATGTAACTGATTGCT | 250 nm | HPLC | 137 |
| splint6 | GTACGCATCACACGAGTATGGAAGTCGTTCTTATCGGAGG | | | |
| | GACCCTAACCCTAACCCTA | | | |
| AscI_NB16_ | GAGTCCACCATGAATTCGGCGCGCCATGTAACTGATTGCT | 250 nm | HPLC | 138 |
| corrected_ | GTACGCAAGTACGAACCACTGTCAGTTGACGTATCGGAGG | | | |
| Splint1 | GCCCTAACCCTAACCCTAA | | | |
| AscI_NB16_ | GAGTCCACCATGAATTCGGCGCGCCATGTAACTGATTGCT | 250 nm | HPLC | 139 |
| corrected_ | GTACGCAAGTACGAACCACTGTCAGTTGACGTATCGGAGG | | | |
| Splint2 | GCCTAACCCTAACCCTAAC | | | |
| AscI_NB16_ | GAGTCCACCATGAATTCGGCGCGCCATGTAACTGATTGCT | 250 nm | HPLC | 140 |
| corrected_ | GTACGCAAGTACGAACCACTGTCAGTTGACGTATCGGAGG | | | |
| Splint3 | GCTAACCCTAACCCTAACC | | | |
| AscI_NB16_ | GAGTCCACCATGAATTCGGCGCGCCATGTAACTGATTGCT | 250 nm | HPLC | 141 |
| corrected_ | GTACGCAAGTACGAACCACTGTCAGTTGACGTATCGGAGG | | | |
| Splint4 | GTAACCCTAACCCTAACCC | | | |
| AscI_NB16_ | GAGTCCACCATGAATTCGGCGCGCCATGTAACTGATTGCT | 250 nm | HPLC | 142 |
| corrected_ | GTACGCAAGTACGAACCACTGTCAGTTGACGTATCGGAGG | | | |
| Splint5 | GAACCCTAACCCTAACCCT | | | |
| AscI_NB16_ | GAGTCCACCATGAATTCGGCGCGCCATGTAACTGATTGCT | 250 nm | HPLC | 143 |
| corrected_ | GTACGCAAGTACGAACCACTGTCAGTTGACGTATCGGAGG | | | |
| Splint6 | GACCCTAACCCTAACCCTA | | | |

In some embodiments of direct telomere profiling, the nucleic acid sequence that specifically binds to at least a portion of the telomere includes a sequence complementary to the repetitive sequence of the telomere. In some embodiments of direct telomere profiling, the nucleic acid comprises a CCCTAA (SEQ ID NO: 144) sequence.

### Biotin Adapter

In some embodiments of direct telomere profiling, the telomere tagging probe can further include a biotin adapter. As used herein, an “adapter” can refer to a moiety that can be coupled to a polynucleotide sequence. In some embodiments of direct telomere profiling, the biotin adapter includes biotin which has a high affinity or preference to associate or bind to the protein avidin or streptavidin. In some embodiments of direct telomere profiling, the tagged telomere sequence can be separated using biotinylation-streptavidin affinity in any number of methods known to the field (e.g., streptavidin-functionalized beads).

In some embodiments of direct telomere profiling, methods described herein include contacting the tagged telomere sequence with a streptavidin-functionalized bead, wherein the biotin adapter of the tagged telomere sequence binds with the streptavidin-functionalized bead, and recovering the tagged telomere sequence. In some embodiments, the streptavidin-functionalized bead is formulated for DNA capture. In some embodiments, the streptavidin-functionalized bead comprises MyOne™ Streptavidin C1 beads.

In some embodiments of direct telomere profiling, the recovering comprises using a magnet to separate the tagged telomere sequence bound to the streptavidin-functionalized bead. In some embodiments of direct telomere profiling, the recovering further comprises releasing the tagged telomere sequence from the streptavidin-functionalized bead using a restriction enzyme (e.g., restriction endonuclease), wherein the restriction enzyme cleaves the tagged telomere sequence at a restriction enzyme-specific site. In some embodiments of direct telomere profiling, the restriction enzyme can include EcoR1 or Asc1. In some embodiments, the restriction enzyme can include ClaI, PvuI, AsiSI, PacI, or PmeI. In some embodiments, the recovering comprises using an RNase to separate the tagged telomere sequence bound to the streptavidin-functionalized bead, wherein the telomere tagging sequence comprises a RNA/DNA hybrid. In some embodiments, the recovering comprises using an endonuclease enzyme to separate the tagged telomere sequence bound to the streptavidin-functionalized bead. In some embodiments, the tagged telomere can include a poly(U) sequence which can be cleaved by a mixture of Uracil DNA glycosylase (UDG) and the DNA glycosylase-lyase Endonuclease VIII, commercially known as the USER™ enzyme. In some embodiments, the recovering can include using heat to separate the tagged telomere sequence bound to the streptavidin-functionalized bead. In some embodiments, the recovering can further include heated elution.

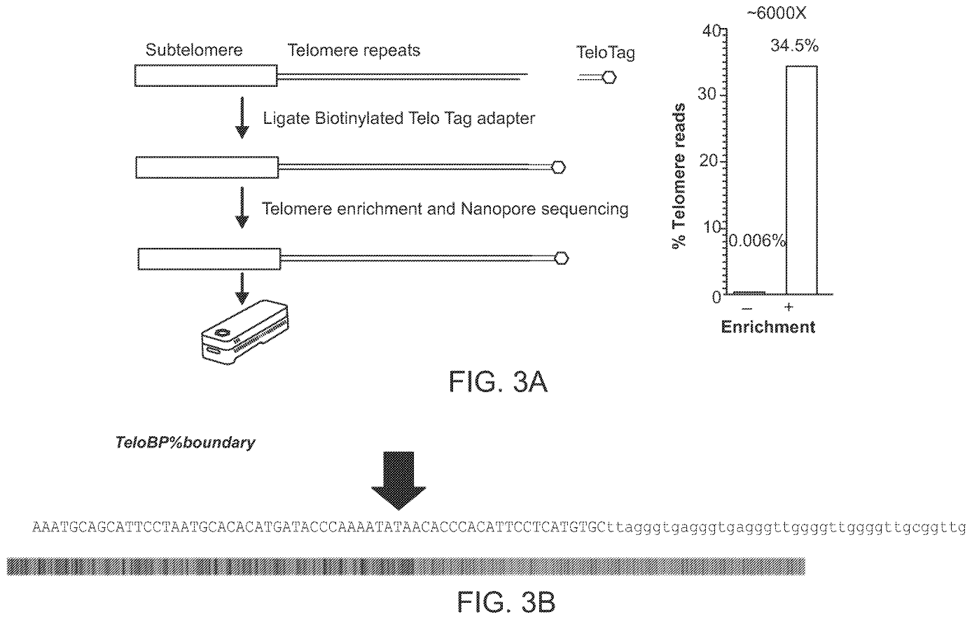

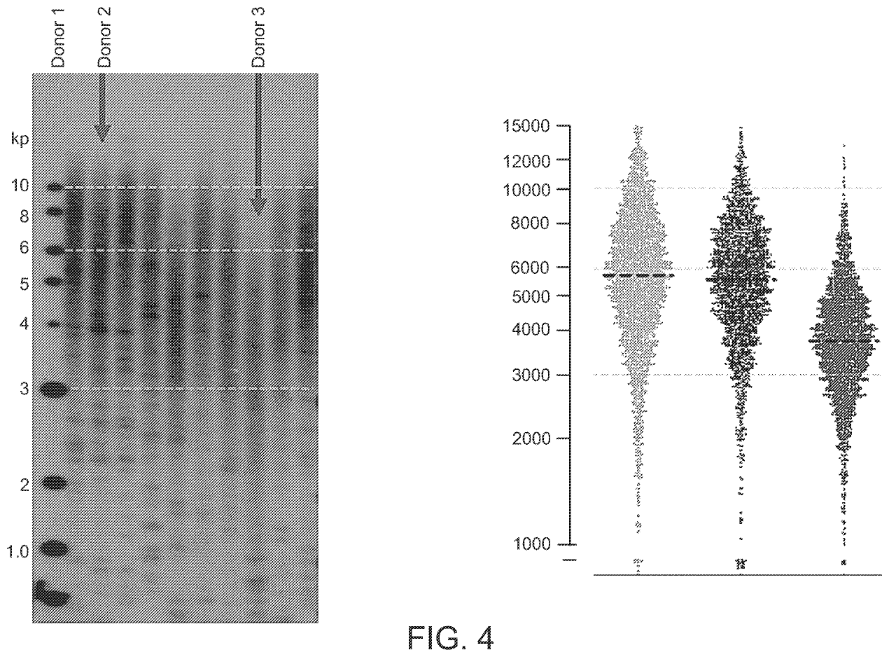

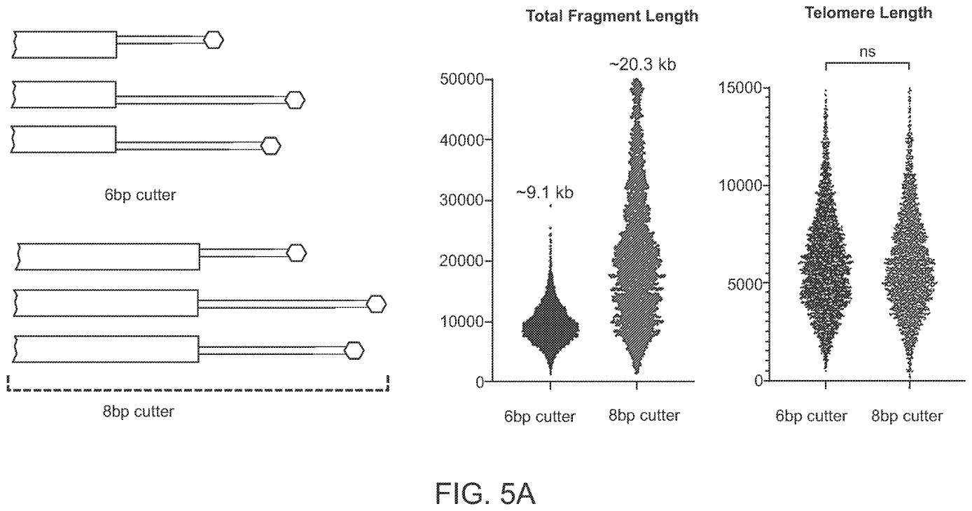

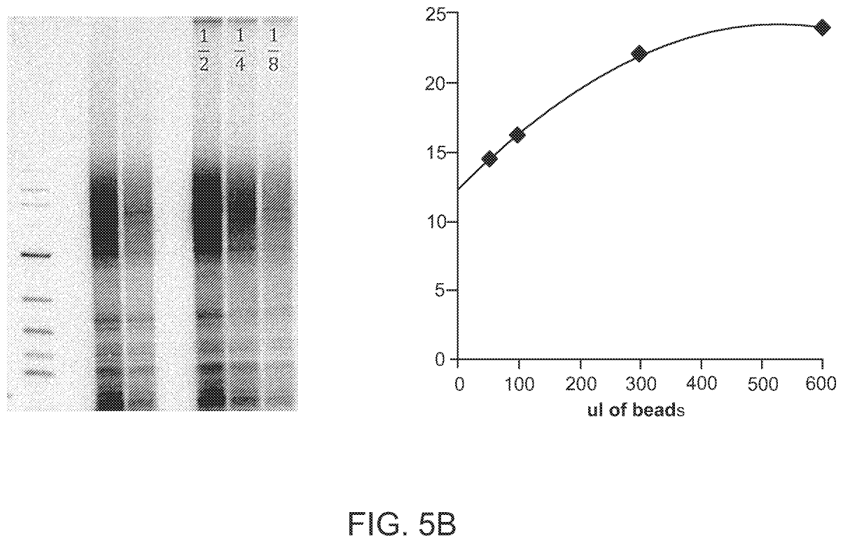

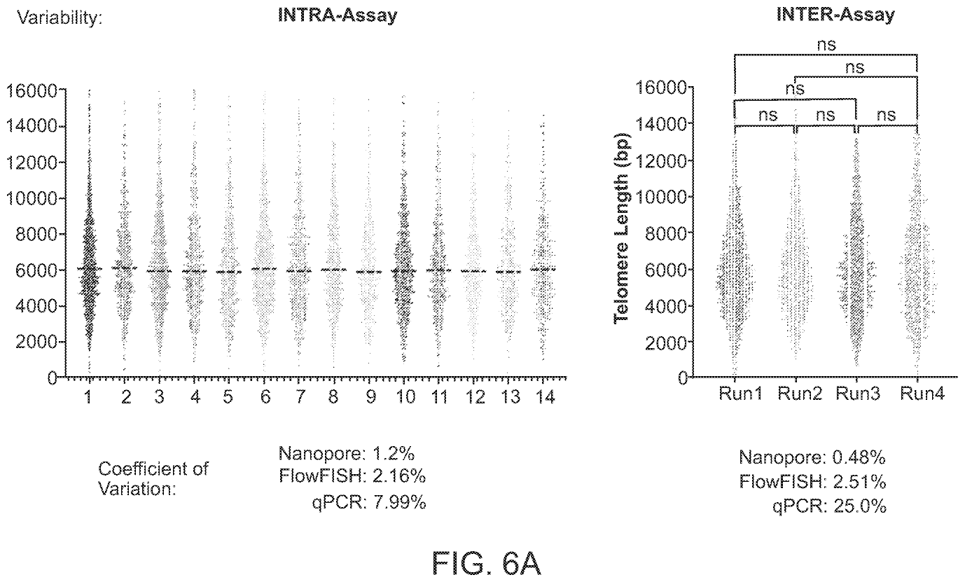

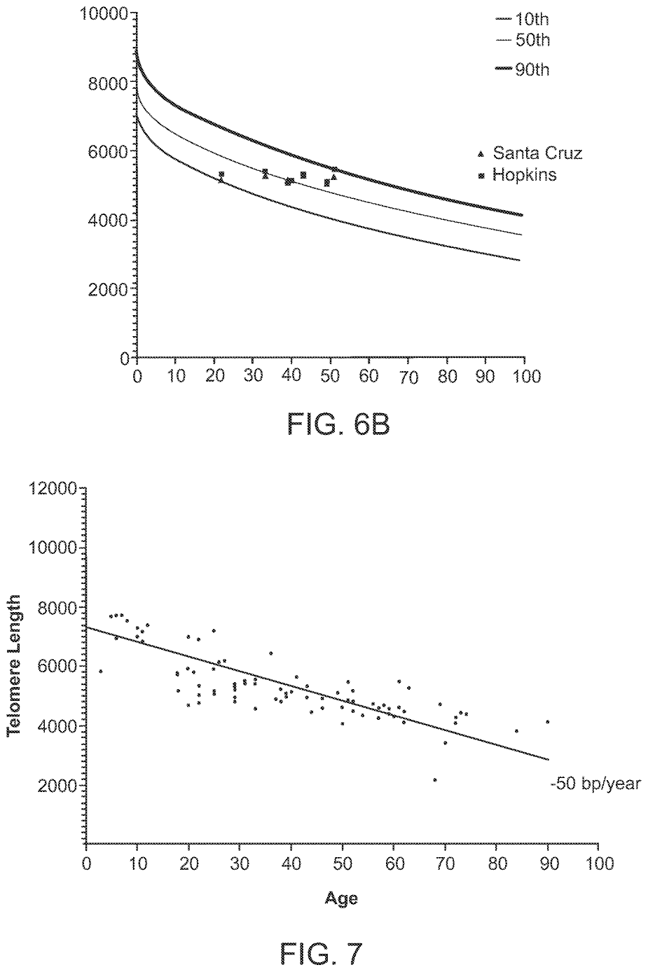

In some embodiments of direct telomere profiling, methods described herein can produce increased telomere enrichment over standard whole genome sequencing (FIG. 3A). In some embodiments of direct telomere profiling, methods described herein can achieve improved telomere length measurement as compared to conventional assays (e.g., standard whole genome sequencing). In some embodiments of direct telomere profiling, improved telomere length measurement can be achieved by the Direct Telomere Profiling method described herein. In some embodiments of direct telomere profiling, methods described herein can reproduce telomere length measurements from telomere length measurements by Southern Blotting (FIG. 4). In some embodiments of direct telomere profiling, methods described herein can produce telomere length measurements without the length bias of nanopore sequencing (FIG. 5A). In some embodiments of direct telomere profiling, methods described herein can reproduce telomere profiling results with lower variability compared to other sequencing methods (FIGS. 6A and 6B).

In some embodiments of direct telomere profiling, methods provided herein can achieve accurate telomere length measurement using small amounts of DNA (e.g., smaller than would conventionally be required). In some embodiments of direct telomere profiling, about 5 to about 10 (e.g., about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 9 to about 10) micrograms of DNA can be used to achieve accurate telomere length measurement.

In some embodiments of direct telomere profiling, methods provided herein can be used in multiplex format. In some embodiments of direct telomere profiling, methods provided herein can be used in multiplex format for screening purposes. In some embodiments of direct telomere profiling, methods provided herein can be used in multiplex format for screening purposes wherein more than 10 (e.g., more than 20, more than 30, more than 40, more than 50, more than 70, or more than 100) samples are tested simultaneously. In some embodiments of direct telomere profiling, methods provided herein can be used in multiplex format for screening purposes wherein more than 100 (e.g., more than 200, more than 300, more than 400, more than 500, more than 600, more than 700, more than 800, more than 900, or more than 1000) samples are tested simultaneously. In some embodiments of direct telomere profiling, methods provided herein can be used to assign chromosome status to the telomere reads. In some embodiments of direct telomere profiling, methods provided herein can identify conserved trends such that telomeres from some chromosome ends (e.g., 8q, 20q, 17q, 19p, 8p, 22q, 17p, and/or 3q arm) have shorter lengths in the general population while other telomeres from other chromosome ends (e.g., 3p, 1p, and/or 4q arm) tend to be longer in most individuals.

### Amplified Telomere Profiling

### Telomere Tagging Probe

As used herein, a “probe” and “tagging probe” can refer to any molecule capable of capturing (directly or indirectly) and/or labelling an analyte (e.g., DNA molecule) in a biological sample. In some embodiments, the tagging probe is a nucleic acid or a polypeptide. In some embodiments of amplified telomere profiling, the tagging probe includes a barcode (e.g., a unique molecular identifier (UMI)). In some embodiments of amplified telomere profiling, a tagging probe can be coupled to a nucleic acid sequence using any one of many different techniques including, but not limited to, ligation, hybridization, and tagmentation. In some embodiments of amplified telomere profiling, a tagging probe can include nucleic acid sequences that add a function, e.g., spacer sequences, primer sequences/sites, and/or sample barcode sequences. In some embodiments, a telomere tagging probe can include a plurality of nucleic acid sequences, wherein the plurality of nucleic acid sequences can be attached to each other via ligation of the nucleic acids.

In some embodiments of amplified telomere profiling, methods described herein include attaching a telomere tagging probe to a 3′ end of the telomere to generate a tagged telomere sequence. In some embodiments, the attaching step comprises ligating the telomere tagging probe to the 3′ end of the telomere. In some embodiments, the ligating comprises cycling ligation. In some embodiments, the ligating comprises using a DNA ligase enzyme. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For example, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase), and Ampligase®. Derivatives, e.g., sequence-modified derivatives, and/or mutants thereof, can also be used. In some embodiments, the ligating comprises a Taq DNA ligase.

### Splint Oligonucleotide

In some embodiments of amplified telomere profiling, methods provided herein include use of a splint oligonucleotide comprising a nucleic acid sequence that specifically binds to at least a portion of the telomere and to at least a portion of the telomere tagging probe. In some embodiments of amplified telomere profiling, the splint oligonucleotide is DNA or RNA. In some embodiments of amplified telomere profiling, the splint oligonucleotide can include a nucleotide sequence that is partially complementary to nucleotide sequences from two or more different oligonucleotides. In general, an RNA ligase, a DNA ligase, or another other variety of ligase is used to ligate two nucleotide sequences together.

In some embodiments of amplified telomere profiling, the splint oligonucleotide is between 10 and 50 oligonucleotides in length (e.g., between 10 and 45, between 10 and 40, between 10 and 35, between 10 and 30, between 10 and 25, between 10 and 20, or between 10 and 15, between 15 and 50, between 15 and 45, between 15 and 40, between 15 and 35, between 15 and 30, between 15 and 25, between 15 and 20, between 20 and 50, between 20 and 45, between 20 and 40, between 20 and 35, between 20 and 30, between 20 and 25, between 25 and 50, between 25 and 45, between 25 and 40, between 25 and 35, between 25 and 30, between 30 and 50, between 30 and 45, between 30 and 40, between 30 and 35, between 35 and 50, between 35 and 45, between 35 and 40, between 40 and 50, between 40 and 45, or between 45 and 50 oligonucleotides in length).

In some embodiments of amplified telomere profiling, a method for determining a telomere length of a DNA molecule from a biological sample can include (a) providing a telomere tagging probe, wherein the telomere tagging probe comprises a unique molecular identifier (UMI) sequence; (b) providing a splint oligonucleotide, wherein the splint oligonucleotide specifically binds to at least a portion of a telomere on the DNA molecule and to at least a portion of the telomere tagging probe; (c) attaching the telomere tagging probe to the telomere to generate a tagged telomere sequence; (d) contacting the tagged telomere sequence with a forward primer and a reverse primer, wherein the forward primer binds to a subtelomere of the telomere, and the reverse primer binds to at least a portion of the telomere tagging probe; (e) amplifying the tagged telomere sequence to generate an amplified tagged telomere sequence; and (f) analyzing the amplified tagged telomere sequence. In some embodiments, the splint oligonucleotide is blocked at its 3′ end. In some embodiments, the splint oligonucleotide is not blocked at its 3′ end.

In some embodiments, amplifying step (e) further comprises generating a forward amplified tagged telomere sequence and a reverse amplified tagged telomere sequence, wherein the forward amplified tagged telomere sequence is generated by an amplification reaction using the forward primer, and wherein the reverse amplified tagged telomere sequence is generated by an amplification reaction using the reverse primer.

In some embodiments of amplified telomere profiling, the splint oligonucleotide is blocked, such that the blocked splint oligonucleotide is a splint oligonucleotide that is blocked at the 3′ end such that it cannot be extended by a nucleic acid polymerase. In some embodiments, a blocked splint oligonucleotide can include a 3′ end modification (e.g., a 3′ dideoxy C (3′ddC), 3′ddG, 3′ddA, 3′ddT, 3′ inverted dT, 3′ C3 spacer, 3′ amino, 3′ biotinylation, or 3′ phosphorylation). In some embodiments, the 3′ end modification can include, but is not limited to, an affinity plus modified base (e.g., locked nucleic acids), 2′-O-methoxy-ethyl Base (2′-MOE), 2′-O-Methyl RNA base, fluoro base (e.g., fluoro C, fluoro U, fluoro A, or fluoro G), 2-aminopurine, 5-bromo dU, deoxyuridine, 2,6-diaminopurine (2-amino-dA), dideoxy-C, deoxylnosine, hydroxymethyl dC, inverted dT, iso-dG, iso-dC, inverted dideoxy-T, 5-methyl dC, or 5-nitroindole). For example, a blocked splint oligonucleotide can include a 3′ inverted dT modification (3invDT).

In some embodiments, amplifying step (e) further comprises generating a forward amplified tagged telomere sequence, wherein the forward amplified tagged telomere sequence is generated by an amplification reaction using the forward primer. In some embodiments, the 3′ end modification of the blocked splint oligonucleotide prevents an amplification reaction using the reverse primer.

In some embodiments of amplified telomere profiling, the nucleic acid sequence that specifically binds to at least a portion of the telomere includes a sequence complementary to the repetitive sequence of the telomere. In some embodiments of amplified telomere profiling, the nucleic acid comprises a CCCTAA (SEQ ID NO: 144) sequence.

### Unique Molecular Identifier (UMI)

In some embodiments of amplified telomere profiling, the telomere tagging probe can include a unique molecular identifier (UMI). As used herein, a “unique molecular identifier” can refer to a nucleic acid segment that functions as a label or identifier for a particular analyte (e.g., a DNA molecule). In some embodiments, a UMI can include one or more random nucleic acid sequences, and/or one or more synthetic nucleic acid sequences, or combinations thereof.

In some embodiments of amplified telomere profiling, methods described herein include contacting the tagged telomere sequence with a forward primer and a reverse primer, wherein the forward primer binds to a subtelomere of the telomere, and the reverse primer binds to at least a portion of the telomere tagging probe, and performing amplification of the tagged telomere sequence. In some embodiments of amplified telomere profiling, amplification of the tagged telomere sequence comprises PCR amplification. In some embodiments of amplified telomere profiling, amplification comprises performing 20 rounds of PCR amplification. In some embodiments of amplified telomere profiling, amplification comprises performing about 15 to about 35 (e.g., about 20 to about 35, about 25 to about 35, about 30 to about 35, about 15 to about 30, about 20 to about 30, about 25 to about 30, about 15 to about 25, about 20 to about 25, or about 15 to about 20) rounds of PCR amplification.

In some embodiments of amplified telomere profiling, methods described herein include analyzing the tagged telomere sequence, thereby determining the telomere length of the DNA molecule. In some embodiments, the telomere tagging probe can further include a sample barcode sequence, wherein the sample barcode sequence is associated with the biological sample. In some embodiments, the sample barcode sequence can be used in combination with the tagged telomere sequence for multiplexing applications.

In some embodiments of amplified telomere profiling, the tagged telomere sequence that includes the UMIs and/or barcoded sequences can be analyzed via sequencing. In some embodiments of amplified telomere profiling, the tagged telomere sequence can be analyzed by using various sequencing systems. In some embodiments of amplified telomere profiling, sequencing can be performed by various commercial systems. In some embodiments of amplified telomere profiling, sequencing can be performed by non-commercialized sequencing systems. Examples of such sequencing systems and techniques can include, but are not limited to, PacBio SMRT sequencing, and Oxford Nanopore sequencing or other long read sequencing methods. More generally, sequencing can be performed using nucleic acid amplification, polymerase chain reaction (PCR) (e.g., digital PCR and droplet digital PCR (ddPCR), multiplex PCR, PCR-based singleplex methods, emulsion PCR), and/or isothermal amplification.

In some embodiments of amplified telomere profiling, other examples of methods for sequencing genetic material include, but are not limited to, DNA hybridization methods (e.g., Southern blotting), restriction enzyme digestion methods, next-generation sequencing methods (e.g., single-molecule real-time sequencing, and nanopore sequencing), and ligation methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, MS-PET sequencing, and any combinations thereof. In some embodiments, sequencing can comprise nanopore sequencing. In some embodiments, sequencing comprises long read sequencing.

### EXAMPLES

The disclosure is further described in the following examples, which do not limit the scope of the disclosure described in the claims.

### Example 1—Nanopore Telomere Profiling Through Biotin Pulldown and Direct Sequencing

### Biotin Adapters

The adapters include a set of 6 splints, each with one of the 6 permutations for the telomere register 3×(CCCTAA). The 3×(CCCTAA) complementary region binds to the 3′ overhang found in all telomeres, while the unique adapter region is a perfect complement (splint) to the biotin adapter that is then ligated to the G strand telomere.

Adapters for bulk telomere pull down (Cutting with a 6-base cutter (EcoRI) to release from beads leaving ~4 kb of subtelomnere) are shown below.

NB10 barcode set:

| | | | | SEQ |
|---|---|---|---|---|
| | | | Puri- | ID |
| Name | Sequence | Scale | fication | NO: |
| NB10- | /5Phos/CCCTCCGATAGAGAGGACAAAGGTTTCAACGCTTTGCGTACAGCA | 1 um | HPLC | 145 |
| Phospho | ATCAGTTACATTGTCAGTGGAATTCATGGTGGACTCTATGACCGTTCAG/ | | | |
| Bio | 3BioTEG/ | | | |
| NB10_ | GAGTCCACCATGAATTCCACTGACAATGTAACTGATTGCTGTACGCAAAG | 1 um | HPLC | 146 |
| indi_splint | CGTTGAAACCTTTGTCCTCTCTATCGGAGGGCCCTAACCCTAACCCTAA/ | | | |
| 1 | 3InvdT/ | | | |
| NB10_ | GAGTCCACCATGAATTCCACTGACAATGTAACTGATTGCTGTACGCAAAG | 1 um | HPLC | 147 |
| indi_splint | CGTTGAAACCTTTGTCCTCTCTATCGGAGGGCCTAACCCTAACCCTAAC/ | | | |
| 2 | 3InvdT/ | | | |
| NB10_ | GAGTCCACCATGAATTCCACTGACAATGTAACTGATTGCTGTACGCAAAG | 1 um | HPLC | 148 |
| indi_splint | CGTTGAAACCTTTGTCCTCTCTATCGGAGGGCTAACCCTAACCCTAACC/ | | | |
| 33I | nvdT/ | | | |
| NB10_ | GAGTCCACCATGAATTCCACTGACAATGTAACTGATTGCTGTACGCAAAG | 1 um | HPLC | 149 |
| indi_splint | CGTTGAAACCTTTGTCCTCTCTATCGGAGGGTAACCCTAACCCTAACCC/ | | | |
| 4 | 3InvdT/ | | | |
| NB10_ | GAGTCCACCATGAATTCCACTGACAATGTAACTGATTGCTGTACGCAAAG | 1 um | HPLC | 150 |
| indi_splint | CGTTGAAACCTTTGTCCTCTCTATCGGAGGGAACCCTAACCCTAACCCT/ | | | |
| 5 | 3InvdT/ | | | |
| NB10_ | GAGTCCACCATGAATTCCACTGACAATGTAACTGATTGCTGTACGCAAAG | 1 um | HPLC | 151 |
| indi_splint | CGTTGAAACCTTTGTCCTCTCTATCGGAGGGACCCTAACCCTAACCCTA/ | | | |
| 6 | 3InvdT/ | | | |

NB12 barcode set:

| | | | | SEQ |
|---|---|---|---|---|
| | | | Puri- | ID |
| Name | Sequence | Scale | fication | NO: |
| NB12- | /5Phos/CCCTCCGATATCCGATTCTGCTTCTTTCTACCTGTGCGTACAGCAA | 1 um | HPLC | 152 |
| PhosphoBiot | TCAGTTACATTGTCAGTGGAATTCATGGTGGACTCTATGACCGTTCAG/ | | | |
| | 3BioTEG/ | | | |
| NB12_indi_ | GAGTCCACCATGAATTCCACTGACAATGTAACTGATTGCTGTACGCACA | 1 um | HPLC | 153 |
| splint1 | GGTAGAAAGAAGCAGAATCGGATATCGGAGGGCCCTAACCCTAACCCT | | | |
| | AA/3InvdT/ | | | |
| NB12_indi_ | GAGTCCACCATGAATTCCACTGACAATGTAACTGATTGCTGTACGCAAA | 1 um | HPLC | 154 |
| splint2 | GCGTTGAAACCTTTGTCCTCTCTATCGGAGGGCCTAACCCTAACCCTAAC | | | |
| | /3InvdT/ | | | |
| NB12_indi_ | GAGTCCACCATGAATTCCACTGACAATGTAACTGATTGCTGTACGCAAA | 1 um | HPLC | 155 |
| splint3 | GCGTTGAAACCTTTGTCCTCTCTATCGGAGGGCTAACCCTAACCCTAACC | | | |
| | /3InvdT/ | | | |
| NB12_indi_ | GAGTCCACCATGAATTCCACTGACAATGTAACTGATTGCTGTACGCAAA | 1 um | HPLC | 156 |
| splint4 | GCGTTGAAACCTTTGTCCTCTCTATCGGAGGGTAACCCTAACCCTAACCC | | | |
| | /3InvdT/ | | | |
| NB12_indi_ | GAGTCCACCATGAATTCCACTGACAATGTAACTGATTGCTGTACGCAAA | 1 um | HPLC | 157 |
| splint5 | GCGTTGAAACCTTTGTCCTCTCTATCGGAGGGAACCCTAACCCTAACCCT | | | |
| | /3InvdT/ | | | |
| NB12_indi_ | GAGTCCACCATGAATTCCACTGACAATGTAACTGATTGCTGTACGCAAA | 1 um | HPLC | 158 |
| splint6 | GCGTTGAAACCTTTGTCCTCTCTATCGGAGGGACCCTAACCCTAACCCTA | | | |
| | /3InvdT/ | | | |

NB13 barcode set:

| | | | | SEQ |
|---|---|---|---|---|
| | | | Puri- | ID |
| Name | Sequence | Scale | fication | NO: |
| NB13- | /5Phos/CCCTCCGATAAGAACGACTTCCATACTCGTGTGATGCGTACAGCA | 1 um | HPLC | 159 |
| PhosphoBiot | ATCAGTTACATTGTCAGTGGAATTCATGGTGGACTCTATGACCGTTCAG/ | | | |
| | 3BioTEG/ | | | |
| NB13_indi_ | GAGTCCACCATGAATTCCACTGACAATGTAACTGATTGCTGTACGCATC | 1 um | HPLC | 160 |
| splint1 | ACACGAGTATGGAAGTCGTTCTTATCGGAGGGCCCTAACCCTAACCCTA | | | |
| | A/3InvdT/ | | | |
| NB13_indi_ | GAGTCCACCATGAATTCCACTGACAATGTAACTGATTGCTGTACGCATC | 1 um | HPLC | 161 |
| splint2 | ACACGAGTATGGAAGTCGTTCTTATCGGAGGGCCTAACCCTAACCCTAA | | | |
| | C/3InvdT/ | | | |
| NB13_indi_ | GAGTCCACCATGAATTCCACTGACAATGTAACTGATTGCTGTACGCATC | 1 um | HPLC | 162 |
| splint3 | ACACGAGTATGGAAGTCGTTCTTATCGGAGGGCTAACCCTAACCCTAAC | | | |
| | C/3InvdT/ | | | |
| NB13_indi_ | GAGTCCACCATGAATTCCACTGACAATGTAACTGATTGCTGTACGCATC | 1 um | HPLC | 163 |
| splint4 | ACACGAGTATGGAAGTCGTTCTTATCGGAGGGTAACCCTAACCCTAACC | | | |
| | C/3InvdT/ | | | |
| NB13_indi_ | GAGTCCACCATGAATTCCACTGACAATGTAACTGATTGCTGTACGCATC | 1 um | HPLC | 164 |
| splint5 | ACACGAGTATGGAAGTCGTTCTTATCGGAGGGAACCCTAACCCTAACCC | | | |
| | T/3InvdT/ | | | |
| NB13_indi_ | GAGTCCACCATGAATTCCACTGACAATGTAACTGATTGCTGTACGCATC | 1 um | HPLC | 165 |
| splint6 | ACACGAGTATGGAAGTCGTTCTTATCGGAGGGACCCTAACCCTAACCCT | | | |
| | A/3InvdT/ | | | |

Adapters for chromosome specific pull downs (Cutting with an 8-base cutter (AscI) to release from beads leaving ~64 kb of subtelomere) are also shown below.

NB16 set:

| | | | | SEQ |
|---|---|---|---|---|
| | | | Puri- | ID |
| Name | Sequence | Scale | fication | NO: |
| Phospho- | /5Phos/CCCTCCGATACGTCAACTGACAGTGGTTCGTACTTGCGTACAGCA | 1 um | HPLC | 166 |
| NB16-Biotin | ATCAGTTACATGGCGCGCCGAATTCATGGTGGACTCTATGACCGTTCAG | | | |
| | AGCT/3BioTEG/ | | | |
| AscI_NB16_ | GAGTCCACCATGAATTCGGCGCGCCATGTAACTGATTGCTGTACGCAAG | 1 um | HPLC | 167 |
| Long_Splint1 | TACGAACCACTGTCAGTTGACGTATCGGAGGGCCCTAACCCTAACCCTA | | | |
| | A/3InvdT/ | | | |
| AscI_NB16_ | GAGTCCACCATGAATTCGGCGCGCCATGTAACTGATTGCTGTACGCAAG | 1 um | HPLC | 168 |
| Long_Splint2 | TACGAACCACTGTCAGTTGACGTATCGGAGGGCCTAACCCTAACCCTAA | | | |
| | C/3InvdT/ | | | |
| AscI_NB16_ | GAGTCCACCATGAATTCGGCGCGCCATGTAACTGATTGCTGTACGCAAG | 1 um | HPLC | 169 |
| Long_Splint3 | TACGAACCACTGTCAGTTGACGTATCGGAGGGCTAACCCTAACCCTAAC | | | |
| | C/3InvdT/ | | | |
| AscI_NB16_ | GAGTCCACCATGAATTCGGCGCGCCATGTAACTGATTGCTGTACGCAAG | 1 um | HPLC | 170 |
| Long_Splint4 | TACGAACCACTGTCAGTTGACGTATCGGAGGGTAACCCTAACCCTAACC | | | |
| | C/3InvdT/ | | | |
| AscI_NB16_ | GAGTCCACCATGAATTCGGCGCGCCATGTAACTGATTGCTGTACGCAAG | 1 um | HPLC | 171 |
| Long_Splint5 | TACGAACCACTGTCAGTTGACGTATCGGAGGGAACCCTAACCCTAACCC | | | |
| | T/3InvdT/ | | | |
| AscI_NB16_ | GAGTCCACCATGAATTCGGCGCGCCATGTAACTGATTGCTGTACGCAAG | 1 um | HPLC | 172 |
| Long_Splint6 | TACGAACCACTGTCAGTTGACGTATCGGAGGGACCCTAACCCTAACCCT | | | |
| | A/3InvdT/ | | | |

NB19 set:

| | | | | SEQ |
|---|---|---|---|---|
| | | | Puri- | ID |
| Name | Sequence | Scale | fication | NO: |
| Phospho- | /5Phos/CCCTCCGATAGTTCCTCGTGCAGTGTCAAGAGATTGCGTACAGCA | 1 um | HPLC | 173 |
| NB19-Biotin | ATCAGTTACATGGCGCGCCGAATTCATGGTGGACTCTATGACCGTTCAG | | | |
| | AGCT/3BioTEG/ | | | |
| AscI_NB19_ | GAGTCCACCATGAATTCGGCGCGCCATGTAACTGATTGCTGTACGCAAT | 1 um | HPLC | 174 |
| Long_Splintl | CTCTTGACACTGCACGAGGAACTATCGGAGGGCCCTAACCCTAACCCTA | | | |
| | A/3InvdT/ | | | |
| AscI_NB19_ | GAGTCCACCATGAATTCGGCGCGCCATGTAACTGATTGCTGTACGCAAT | 1 um | HPLC | 175 |
| Long_Splint2 | CTCTTGACACTGCACGAGGAACTATCGGAGGGCCTAACCCTAACCCTAA | | | |
| | C/3InvdT/ | | | |
| AscI_NB19_ | GAGTCCACCATGAATTCGGCGCGCCATGTAACTGATTGCTGTACGCAAT | 1 um | HPLC | 176 |
| Long_Splint3 | CTCTTGACACTGCACGAGGAACTATCGGAGGGCTAACCCTAACCCTAAC | | | |
| | C/3InvdT/ | | | |
| AscI_NB19_ | GAGTCCACCATGAATTCGGCGCGCCATGTAACTGATTGCTGTACGCAAT | 1 um | HPLC | 177 |
| Long_Splint4 | CTCTTGACACTGCACGAGGAACTATCGGAGGGTAACCCTAACCCTAACC | | | |
| | C/3InvdT/ | | | |
| AscI_NB19_ | GAGTCCACCATGAATTCGGCGCGCCATGTAACTGATTGCTGTACGCAAT | 1 um | HPLC | 178 |
| Long_Splint5 | CTCTTGACACTGCACGAGGAACTATCGGAGGGAACCCTAACCCTAACCC | | | |
| | T/3InvdT/ | | | |
| AscI_NB19_ | GAGTCCACCATGAATTCGGCGCGCCATGTAACTGATTGCTGTACGCAAT | 1 um | HPLC | 179 |
| Long_Splint6 | CTCTTGACACTGCACGAGGAACTATCGGAGGGACCCTAACCCTAACCCT | | | |
| | A/3InvdT/ | | | |

Annealing Biotin Adapters with Splints

The oligonucleotides were annealed together to make a duplex adapter set that has both the splint and adapter hybridized together. This was done in volume of 100 μLs in a PCR tube. An adapter mix that has 30 μM of phosphorylated UMI adapter and 5 μM of each splint permutation was made. The taq ligase buffer has high salt and was used for ensuring that the duplex DNA is stable at high temperatures once formed. The Taq ligation buffer was heated to 65 °C to dissolve the salts.

Using a thermocycler, the oligos were annealed, then first denatured at 95 °C and cooled by decreasing the temperature by 1 Degree®C/min until the temperature reached predicted Tm (85 °C). The adapter mix was then held at the Tm for 10 mins. After this, the adapter mix was cooled to 4 °C by cooling 1 °C/min. The annealed adapters were then held at 4 degrees.

Ligation of Biotin TeloTag adapters to HMW DNA

A 1:100 dilution for the annealed adapters mix was made in 1× hifi TAQ buffer. The taq buffer was heated and then diluted 10 μl of buffer with 90 μL of water. The solution was cooled down by placing on ice. Then, 99 μL of this 1× hifi Buffer was taken and 1 μL of the annealed adapter mix was added. The components were then mixed in a protein lo-bind tube on ice and the tubes were placed in thermocycler and a cycling ligation program was run, wherein the program heats to 70 °C and denatures the DNA just enough to make sure the 3′ telomere overhang is not forming any secondary structures. The thermocycler then slowly cools to 45 °C where the ligation takes place. This was repeated for 15 cycles to allow for binding of the different permutations of the telomere adapter to capture all telomeres.

Cleanup of HMW DNA with Tagged Telomeres Using and Removing Excess Adapters Using SPRI Select Beads

It was needed to clean up the DNA to remove all the excess adapters that were in solution and were not ligated. The SPRI Select beads bottle was placed on the hula mixer rotating at 3 rpm for 1 hour. 1× cutsmart was made from 10× stock and warmed to 37 °C. The PCR tubes were then spun down from the previous step to collect all the liquid. The ligated reaction was pulled in a 1.5 mL protein lo bind tube. The ligated reaction was then dispensed to a new tube. The beads and DNA was mixed with wide orifice tips, mix 20 times, steadily, and the tube was placed on hula at rotating at 10 rpm for 7 mins.

The tube was then placed on magnet for 1 hr and 30 min. The supernatant very gently pipetted off and washed 2× with freshly made 85% ethanol. The Ethanol was taken off, spun down using a microcentrifuge, and placed back on magnet and any trace amounts of ethanol was removed using a P20 pipette. 200 μl (per 40 μg of input DNA) of the warmed 1× cutsmart was added, the tube was then removed from magnet and mix gently with rainin pipette tip (not wide orifice) for 20 times to break up the bead/DNA complex. The pooled samples were incubated at 37 °C for 15 mins and placed on magnet for 2 min. The amount of DNA was then measured using Qubit BR (1 μl of DNA).

### Restriction Digest of Biotin Tagged and Purified DNA Product

Since the streptavidin beads cannot bind to HMW DNA, it was needed to partially digest the DNA. The type of restriction enzyme used depends on how much of the subtelomere that would need to be captured. For bulk telomere sequencing the 6-base cutter BamHI was used that leaves ~4 kb of subtelomere sequence. EcoRI was used to release the adapters from streptavidin beads at a later step. If longer subtelomere sequences are needed (for chromosome specific assignment of telomeres), then an 8 base cutter can be used (e.g., Not1 or Asc1) with the biotin adapters that have a Not1 or Asc1 cut site instead. The tradeoff here is that fewer molecules are pulled down compared to the 6 bp cutter due to the properties of the streptavidin beads.

3 ul of EcoR1 HF (100,000 U/mL) was added to the purified tagged DNA in cutsmart, then digested for 15-30 min at 37 °C. For chromosome specific analysis, 3 μL of Asc1 was used.

### Biotin Pulldown of Tagged Telomeres Using Magnetic Streptavidin Beads

The M-280 Streptavidin beads and binding solution were brought to room temp, and the bottle was placed on HuLa rotating at 3 rpm. 50 ul of M-280 Streptavadin beads were taken and placed in 1.5 ml EP Eppendorf tube and placed on magnet for 2 min. 50 μl of supernatant was then taken off and afterwards the tube was taken off the magnet and resuspended in 200 μl of binding solution. At this point the telomeres are being bound to the streptavidin and pipetting can shear the telomere and break off the adapter. The eppendorf tube was placed on a hula (taped horizontally with a slight slant) and incubated at room temp for 3 h at 1 rpm. The samples were placed on the magnet for 1 min or until the supernatant seems clear, then removed from magnet and added on top of beads 400 μl of DynaBeads Washing Solution. The samples were placed on the hula or roller to continue resuspension for 15 min.

A digest solution was prepared to release the telomeres from the streptavidin beads, where the beads were placed back on magnet and 70 μl of digest solution was added to the beads. If multiple samples are being multiplexed for sequencing this is the step to pool them and do a pooled digestion: resuspend 1 sample and then move the bead/digest solution from tube 1 to the next tube to pool and digest the samples together. The samples were incubated for 2 h taped horizontally with a slight angle on hula inside the 37 °C incubator rotating at 2 rpm. The beads were then placed on magnet and the supernatant contains released telomeres.

### Nanopore Library Prep Using the LSK110 kit

The DNA from the digest step was measured using Qubit HSyour DNA, where a total DNA range of about 6-20 ng of DNA was measured in the lo bind tube.

### Example 2—PacBio Telomere Profiling Using PCR Amplification

### PacBio Splints

The splints below hybridize to the G strand and allow for an adapter to get next to the 3′ end of the natural end. The TeloTag is then ligated to this natural end through cycling taq ligase. The GCTACATGCTCCTGTTGTTAGGAGGG (SEQ ID NO: 180) is included in the constant landing pad for all barcodes. The 3′ end can be blocked and have a 3invDT or other non-extendable block as this ensures that the splints do not start amplifying telomeres in the PCR reaction given their 3×CCTAA.

| Name | Sequence | SEQ ID NO: |
|---|---|---|
| WT_ADDIT_UNI_ | GCTACATGCTCCTGTTGTTAGGAGGGCCCTAACCCTAACCCTAA/ | 181 |
| splint1 | 3InvdT/ | |
| WT_ADDIT_UNI_ | GCTACATGCTCCTGTTGTTAGGAGGGCCTAACCCTAACCCTAAC/ | 182 |
| splint2 | 3InvdT/ | |
| WT_ADDIT_UNI_ | GCTACATGCTCCTGTTGTTAGGAGGGCTAACCCTAACCCTAACC/ | 183 |
| splint3 | 3InvdT/ | |
| WT_ADDIT_UNI_ | GCTACATGCTCCTGTTGTTAGGAGGGTAACCCTAACCCTAACCC/ | 184 |
| nt4 | 3InvdT/ | |

spli

| | | |
|---|---|---|
| WT_ADDIT_UNI_ | GCTACATGCTCCTGTTGTTAGGAGGGAACCCTAACCCTAACCCT/ | 185 |
| splint5 | 3InvdT/ | |
| WT_ADDIT_UNI_ | GCTACATGCTCCTGTTGTTAGGAGGGACCCTAACCCTAACCCTA/ | 186 |
| splint6 | 3InvdT/ | |

PacBio TeloTag Mulitplex Adapters with UMI—one of these multiplexing primers can be used with the splints above.

| | | | | SEQ |
|---|---|---|---|---|
| | | | Puri- | ID |
| Name | Sequence | Scale | fication | NO: |
| NNN_NB03 | /Phos/CCCTCCTAACAACAGGAGCATGTAGCNNNYRNNNYRNNNYRNNN | 1 um | HPLC | 187 |
| | CCTGGTAACTGGGACACAAGACTCGCCTCGTCGACTGACAGATGTCAC | | | |
| | GGAGCA | | | |
| NNN_NB20 | /5Phos/CCCTCCTAACAACAGGAGCATGTAGCNNNYRNNNYRNNNYRNN | 1 um | HPLC | 188 |
| | NTTGCGTCCTGTTACGAGAACTCATGCCTCGTCGACTGACAGATGTCAC | | | |
| | GGAGCA | | | |
| NNN_NB21 | /5Phos/CCCTCCTAACAACAGGAGCATGTAGCNNNYRNNNYRNNNYRNN | 1 um | HPLC | 189 |
| | NGAGCCTCTCATTGTCCGTTCTCTAGCCTCGTCGACTGACAGATGTCAC | | | |
| | GGAGCA | | | |
| NNN_NB22 | /5Phos/CCCTCCTAACAACAGGAGCATGTAGCNNNYRNNNYRNNNYRNN | 1 um | HPLC | 190 |
| | NACCACTGCCATGTATCAAAGTACGGCCTCGTCGACTGACAGATGTCA | | | |
| | CGGAGCA | | | |
| NNN_NB26 | /5Phos/CCCTCCTAACAACAGGAGCATGTAGCNNNYRNNNYRNNNYRNN | 1 um | HPLC | 191 |
| | NCATACAGCGACTACGCATTCTCATGCCTCGTCGACTGACAGATGTCAC | | | |
| | GGAGCA | | | |
| NNN_NB27 | /5Phos/CCCTCCTAACAACAGGAGCATGTAGCNNNYRNNNYRNNNYRNN | 1 um | HPLC | 192 |
| | NCGACGGTTAGATTCACCTCTTACAGCCTCGTCGACTGACAGATGTCAC | | | |
| | GGAGCA | | | |

Reverse primer to be used for amplifying—the following primer can be used for all of the multiplex sets above for PCR amplification and binds to the 3′ end of the adapters above.

| | | | Puri- | SEQ |
|---|---|---|---|---|
| Name | Sequence | Scale | fication | ID NO: |
| Reverse Primer | TGCTCCGTGACATCTGTCAGTCGACGAGGC | 1 um | HPLC | 193 |
| for PCR | | | | |

### Annealing Oligo Adapters

The oligos were annealed together to make a duplex adapter set that has both the splint and adapter hybridized together. This was done in volume of 100 μLs in a PCR tube. The adapters were mixed to make an adapter mix that has 30 M of phosphorylated UMI adapter and 5 μM of each splint permutation. The taq ligase buffer has high salt and was used for ensuring that the duplex DNA is stable at high temperatures once formed. The Taq ligation buffer was heated to 65 °C to dissolve the salts. A thermocycler was used to anneal the oligos the oligos were first denatured at 95 °C and then cooled by decreasing the temperature by 1 °C/min until the temperature reached predicted Tm (70 °C). The adapter mix was held at Tm for 10 mins, then cooled to 4 °C by cooling 1 °C/min. The mix was then held at 4 degrees.

### Ligation of PacBio Multiplex UMI TeloTag Adapters to HMW DNA

A 1:100 dilution for the annealed adapters mix was made in 1× hifi TAQ buffer. The taq buffer was heated and then diluted 10 μl of buffer with 90 μL of water. The solution was cooled down by placing on ice. Then, 99 μL of this 1× hifi Buffer was taken and 1 μL of the annealed adapter mix was added. The components were then mixed in a protein lo-bind tube on ice and the tubes were placed in thermocycles and a cycling ligation program was run, wherein the program heats to 70 °C and denatures the DNA just enough to make sure the 3′ telomere overhang is not forming any secondary structures. The thermocycle then slowly cools to 45 °C where the ligation takes place. This was repeated for 15 cycles to allow for binding of the different permutations of the telomere adapter to capture all telomeres.

Cleanup of HMW DNA with Tagged Telomeres using and removing excess adapters using SPRI Select Beads

It was needed to clean up the DNA to remove all the excess adapters that were in solution and were not ligated. The SPRI Select beads bottle was placed on the hula mixer rotating at 3 rpm for 1 hour. 1× cutsmart was made from 10× stock and warmed to 37 °C. The PCR tubes were then spun down from the previous step to collect all the liquid. The ligated reaction was pulled in a 1.5 mL protein lo bind tube. The ligated reaction was then dispensed to a new tube. The beads and DNA was mixed with wide orifice tips, mix 20 times, steadily, and the tube was placed on hula at rotating at 10 rpm for 7 mins.

The tube was then placed on magnet for 90 mins. The supernatant very gently pipetted off and washed 2× with freshly made 85% ethanol. The Ethanol was taken off, spun down using a microcentrifuge, and placed back on magnet and any trace amounts of ethanol was removed using a P20 pipette. 200 μl (per 40 μg of input DNA) of the warmed 1× cutsmart buffer was added, the tube was then removed from magnet and mix gently with rainin pipette tip (not wide orifice) for 20 times to break up the bead/DNA complex. The pooled samples were incubated at 37 °C for 15 mins and placed on magnet for 2 min. The amount of DNA was then measured using Qubit BR (1 μl of DNA).

### Restriction Cutting of Tagged and Purified DNA

It was needed to cut the DNA here since PCR amplification would be performed later and HMW would inhibit PCR. The restriction enzyme used is a sequence that should not be either in the subtelomere or the adapters that are ligated. EcoR1 was used, since EcoR1 is compatible with the adapters listed above however other appropriate restriction enzymes could be used instead. 3 μl of EcoR1 HF (100,000 U/mL) was added to the purified tagged DNA in cutsmart and digested for 2 hours at 37 °C.

### PCR Amplification Using XpYp or HYG Adapters

The next step was to PCR amplify a particular telomere of interest. This was done by using a forward primer that that binds to the pseudoautosomal region of the X and Y chromosomes that is the subtelomere and right before the telomere sequence. Other primers to subtelomeres could be used.

Input DNA should be around 20 ng and never more than 100 ng as it can inhibit PCR. It was important to use Failsafe polymerase as other polymerases cannot amplify through the telomeric end.

Cleaning Up PCR Amplified Telomere Products with SPRI PacBio Ampure Beads

PacBio Ampure PB beads were placed on Hula rotating at 3 rpm. After PCR amplification, the samples were spun down where the samples were transferred into a new lo bind tube. At this point, it should be considered whether multiplexing will be performed or not and whether the PCR products are at different lengths. If telomeres are expected to have widely different lengths: equimolar amounts are needed for each DNA sample. It is needed to cleanup each sample separately, quantitate the DNA, calculate equimolar concentrations and then pool the samples. If telomeres are around the same length: the samples can just be pooled at this step all in one tube.

The AMPure PB beads and DNA were mixed and the tube was placed on hula at rotating at 10 rpm for 7 mins. The tube was then placed on magnet for 1:30 mins, and then washed 2× with freshly made 80% ethanol. Then, the tube was taken off magnet and eluted with warmed and well mixed PacBio Elution buffer (55 μL per library prep reaction). The sample was removed from magnet and mix gently with normal rainin pipette tips (not wide orifice) for 20 times to break up the bead/DNA complex. The pooled samples were incubated at 37 °C for 15 mins and placed on magnet for 2 min. A P200 rainin tip was used to suck up the liquid slowly and place on protein low bind tubes, where the amount of DNA was measured using Qubit HS (1 ul of DNA).

### PacBio Library Prep

Single-strand overhangs were removed and the DNA Prep Additive was prepared. The DNA Prep Additive was diluted with Enzyme Dilution Buffer to a total volume of 5 μL. 10.0 μL of the master mix was added to the tube-strips containing 45.0 μL-53.0 μL of sheared DNA. The total volume in this step was 55.0 μL-63.0 μL. A wide bore pipette was used to mix the reaction well by pipetting up and down 20 times, and the contents of the tube strips were then spun down with a quick spin in a microfuge.

To each sample, DNA Damage Repair Mix was added to repair DNA damage, then the samples were treated with End Prep Mix for DNA end repair and A tailing. Adapter ligation was performed by adding Overhang Adapter to each sample, followed by size select of SMRTbell library using LOX AMPure® PB beads, wherein AMPure PB beads were added to the ligation reaction and the DNA was then eluted with elution buffer. The libraries were then sequenced with PacBio's HiFi read technology. PacBio HiFi reads rely on extended run times (30 hours+) to allow ample time for the sequencing polymerase to pass through circularized DNA multiple times (Median of 23 passes in a representative run).

### Example 3—Nanopore Telomere Profiling Through Biotin Pulldown and Direct Sequencing (without 3′ End Modification of the Splint Oligonucleotide)

### Biotin Adapters

The adapters include a set of 6 splints, each with one of the 6 permutations for the telomere register 3×(CCCTAA). The 3×(CCCTAA) complementary region binds to the 3′ overhang found in all telomeres, while the unique adapter region is a perfect complement (splint) to the biotin adapter that is then ligated to the G strand telomeres.

Adapters for bulk telomere pull down (Cutting with a 6-base cutter (EcoRI) to release from beads leaving ~4 kb of subtelomere) are shown below.

NB10 barcode set:

| | | | | SEQ |
|---|---|---|---|---|
| | | | Puri- | ID |
| Name | Sequence | Scale | fication | NO: |
| NB10- | /5Phos/CCCTCCGATAGAGAGGACAAAGGTTTCAACGCTTTGCGTACAGCA | 1 um | HPLC | 145 |
| PhosphoBio | ATCAGTTACATTGTCAGTGGAATTCATGGTGGACTCTATGACCGTTCAG/ | | | |
| | 3BioTEG/ | | | |
| NB10_ | GAGTCCACCATGAATTCCACTGACAATGTAACTGATTGCTGTACGCAAAG | 1 um | HPLC | 146 |
| indi_ | CGTTGAAACCTTTGTCCTCTCTATCGGAGGGCCCTAACCCTAACCCTAA/ | | | |
| splint1 | 3InvdT/ | | | |
| NB10_ | GAGTCCACCATGAATTCCACTGACAATGTAACTGATTGCTGTACGCAAAG | 1 um | HPLC | 147 |
| indi_ | CGTTGAAACCTTTGTCCTCTCTATCGGAGGGCCTAACCCTAACCCTAAC/ | | | |
| splint2 | 3InvdT/ | | | |
| NB10_ | GAGTCCACCATGAATTCCACTGACAATGTAACTGATTGCTGTACGCAAAG | 1 um | HPLC | 148 |
| indi_ | CGTTGAAACCTTTGTCCTCTCTATCGGAGGGCTAACCCTAACCCTAACC/ | | | |
| splint3 | 3InvdT/ | | | |
| NB10_ | GAGTCCACCATGAATTCCACTGACAATGTAACTGATTGCTGTACGCAAAG | 1 um | HPLC | 149 |
| indi_ | CGTTGAAACCTTTGTCCTCTCTATCGGAGGGTAACCCTAACCCTAACCC/ | | | |
| splint4 | 3InvdT/ | | | |
| NB10_ | GAGTCCACCATGAATTCCACTGACAATGTAACTGATTGCTGTACGCAAAG | 1 um | HPLC | 150 |
| indi_ | CGTTGAAACCTTTGTCCTCTCTATCGGAGGGAACCCTAACCCTAACCCT/ | | | |
| splint5 | 3InvdT/ | | | |
| NB10_ | GAGTCCACCATGAATTCCACTGACAATGTAACTGATTGCTGTACGCAAAG | 1 um | HPLC | 151 |
| indi_ | CGTTGAAACCTTTGTCCTCTCTATCGGAGGGACCCTAACCCTAACCCTA/ | | | |
| splint 6 | n3IvdT/ | | | |

NB12 barcode set:

| | | | | SEQ |
|---|---|---|---|---|
| | | | Puri- | ID |
| Name | Sequence | Scale | fication | NO: |
| NB12- | /5Phos/CCCTCCGATATCCGATTCTGCTTCTTTCTACCTGTGCGTACAGCAA | 1 um | HPLC | 152 |
| PhosphoBiot | TCAGTTACATTGTCAGTGGAATTCATGGTGGACTCTATGACCGTTCAG/3 | | | |
| | BioTEG/ | | | |
| NB12_indi_ | GAGTCCACCATGAATTCCACTGACAATGTAACTGATTGCTGTACGCACA | 1 um | HPLC | 153 |
| splint1 | GGTAGAAAGAAGCAGAATCGGATATCGGAGGGCCCTAACCCTAACCCT | | | |
| | AA/3InvdT/ | | | |
| NB12_indi_ | GAGTCCACCATGAATTCCACTGACAATGTAACTGATTGCTGTACGCAAA | 1 um | HPLC | 154 |
| splint2 | GCGTTGAAACCTTTGTCCTCTCTATCGGAGGGCCTAACCCTAACCCTAAC | | | |
| | /3InvdT/ | | | |
| NB12_indi_ | GAGTCCACCATGAATTCCACTGACAATGTAACTGATTGCTGTACGCAAA | 1 um | HPLC | 155 |
| splint3 | GCGTTGAAACCTTTGTCCTCTCTATCGGAGGGCTAACCCTAACCCTAACC | | | |
| | /3InvdT/ | | | |
| NB12_indi_ | GAGTCCACCATGAATTCCACTGACAATGTAACTGATTGCTGTACGCAAA | 1 um | HPLC | 156 |
| splint4 | GCGTTGAAACCTTTGTCCTCTCTATCGGAGGGTAACCCTAACCCTAACCC | | | |
| | /3InvdT/ | | | |
| NB12_indi_ | GAGTCCACCATGAATTCCACTGACAATGTAACTGATTGCTGTACGCAAA | 1 um | HPLC | 157 |
| splint5 | GCGTTGAAACCTTTGTCCTCTCTATCGGAGGGAACCCTAACCCTAACCCT | | | |
| | /3InvdT/ | | | |
| NB12_indi_ | GAGTCCACCATGAATTCCACTGACAATGTAACTGATTGCTGTACGCAAA | 1 um | HPLC | 158 |
| splint6 | GCGTTGAAACCTTTGTCCTCTCTATCGGAGGGACCCTAACCCTAACCCTA | | | |
| | /3TnvdT/ | | | |

NB13 barcode set:

| | | | | SEQ |
|---|---|---|---|---|
| | | | Puri- | ID |
| Name | Sequence | Scale | fication | NO: |
| NB13- | /5Phos/CCCTCCGATAAGAACGACTTCCATACTCGTGTGATGCGTACAGCA | 1 um | HPLC | 159 |
| PhosphoBiot | ATCAGTTACATTGTCAGTGGAATTCATGGTGGACTCTATGACCGTTCAG/ | | | |
| | 3BioTEG/ | | | |
| NB13_indi_ | GAGTCCACCATGAATTCCACTGACAATGTAACTGATTGCTGTACGCATC | 1 um | HPLC | 160 |
| splint1 | ACACGAGTATGGAAGTCGTTCTTATCGGAGGGCCCTAACCCTAACCCTA | | | |
| | A/3InvdT/ | | | |
| NB13_indi_ | GAGTCCACCATGAATTCCACTGACAATGTAACTGATTGCTGTACGCATC | 1 um | HPLC | 161 |
| splint2 | ACACGAGTATGGAAGTCGTTCTTATCGGAGGGCCTAACCCTAACCCTAA | | | |
| | C/3InvdT/ | | | |
| NB13_indi_ | GAGTCCACCATGAATTCCACTGACAATGTAACTGATTGCTGTACGCATC | 1 um | HPLC | 162 |
| splint3 | ACACGAGTATGGAAGTCGTTCTTATCGGAGGGCTAACCCTAACCCTAAC | | | |
| | C/3InvdT/ | | | |
| NB13_indi_ | GAGTCCACCATGAATTCCACTGACAATGTAACTGATTGCTGTACGCATC | 1 um | HPLC | 163 |
| splint4 | ACACGAGTATGGAAGTCGTTCTTATCGGAGGGTAACCCTAACCCTAACC | | | |
| | C/3InvdT/ | | | |
| NB13_indi_ | GAGTCCACCATGAATTCCACTGACAATGTAACTGATTGCTGTACGCATC | 1 um | HPLC | 164 |
| splint5 | ACACGAGTATGGAAGTCGTTCTTATCGGAGGGAACCCTAACCCTAACCC | | | |
| | T/3InvdT/ | | | |
| NB13_indi_ | GAGTCCACCATGAATTCCACTGACAATGTAACTGATTGCTGTACGCATC | 1 um | HPLC | 165 |
| splint6 | ACACGAGTATGGAAGTCGTTCTTATCGGAGGGACCCTAACCCTAACCCT | | | |
| | A/3InvdT/ | | | |

Adapters for chromosome specific pull downs (Cutting with an 8-base cutter (AscI) to release from beads leaving ~64 kb of subtelomere) are also shown below.

NB16 set:

| | | | | SEQ |
|---|---|---|---|---|
| | | | Puri- | ID |
| Name | Sequence | Scale | fication | NO: |
| Phospho- | /5Phos/CCCTCCGATACGTCAACTGACAGTGGTTCGTACTTGCGTACAGCA | 1 um | HPLC | 166 |
| NB16-Biotin | ATCAGTTACATGGCGCGCCGAATTCATGGTGGACTCTATGACCGTTCAG | | | |
| | AGCT/3BioTEG/ | | | |
| AscI_NB16_ | GAGTCCACCATGAATTCGGCGCGCCATGTAACTGATTGCTGTACGCAAG | 1 um | HPLC | 167 |
| Long_Splint1 | TACGAACCACTGTCAGTTGACGTATCGGAGGGCCCTAACCCTAACCCTA | | | |
| | A/3InvdT/ | | | |
| AscI_NB16_ | GAGTCCACCATGAATTCGGCGCGCCATGTAACTGATTGCTGTACGCAAG | 1 um | HPLC | 168 |
| Long_Splint2 | TACGAACCACTGTCAGTTGACGTATCGGAGGGCCTAACCCTAACCCTAA | | | |
| | C/3InvdT/ | | | |
| AscI_NB16_ | GAGTCCACCATGAATTCGGCGCGCCATGTAACTGATTGCTGTACGCAAG | 1 um | HPLC | 169 |
| Long_Splint3 | TACGAACCACTGTCAGTTGACGTATCGGAGGGCTAACCCTAACCCTAAC | | | |
| | C/3InvdT/ | | | |
| AscI_NB16_ | GAGTCCACCATGAATTCGGCGCGCCATGTAACTGATTGCTGTACGCAAG | 1 um | HPLC | 170 |
| Long_Splint4 | TACGAACCACTGTCAGTTGACGTATCGGAGGGTAACCCTAACCCTAACC | | | |
| | C/3InvdT/ | | | |
| AscI_NB16_ | GAGTCCACCATGAATTCGGCGCGCCATGTAACTGATTGCTGTACGCAAG | 1 um | HPLC | 171 |
| Long_Splint5 | TACGAACCACTGTCAGTTGACGTATCGGAGGGAACCCTAACCCTAACCC | | | |
| | T/3InvdT/ | | | |
| AscI_NB16_ | GAGTCCACCATGAATTCGGCGCGCCATGTAACTGATTGCTGTACGCAAG | 1 um | HPLC | 172 |
| Long_Splint6 | TACGAACCACTGTCAGTTGACGTATCGGAGGGACCCTAACCCTAACCCT | | | |
| | A/3InvdT/ | | | |

NB19 set:

| | | | | SEQ |
|---|---|---|---|---|
| | | | Puri- | ID |
| Name | Sequence | Scale | fication | NO: |
| Phospho- | /5Phos/CCCTCCGATAGTTCCTCGTGCAGTGTCAAGAGATTGCGTACAGCA | 1 um | HPLC | 173 |
| NB19-Biotin | ATCAGTTACATGGCGCGCCGAATTCATGGTGGACTCTATGACCGTTCAG | | | |
| | AGCT/3BioTEG/ | | | |
| AscI_NB19_ | GAGTCCACCATGAATTCGGCGCGCCATGTAACTGATTGCTGTACGCAAT | 1 um | HPLC | 174 |
| Long_Splint1 | CTCTTGACACTGCACGAGGAACTATCGGAGGGCCCTAACCCTAACCCTA | | | |
| | A/3InvdT/ | | | |
| AscI_NB19_ | GAGTCCACCATGAATTCGGCGCGCCATGTAACTGATTGCTGTACGCAAT | 1 um | HPLC | 175 |
| Long_Splint2 | CTCTTGACACTGCACGAGGAACTATCGGAGGGCCTAACCCTAACCCTAA | | | |
| | C/3InvdT/ | | | |
| AscI_NB19_ | GAGTCCACCATGAATTCGGCGCGCCATGTAACTGATTGCTGTACGCAAT | 1 um | HPLC | 176 |
| Long_Splint3 | CTCTTGACACTGCACGAGGAACTATCGGAGGGCTAACCCTAACCCTAAC | | | |
| | C/3InvdT/ | | | |
| AscI_NB19_ | GAGTCCACCATGAATTCGGCGCGCCATGTAACTGATTGCTGTACGCAAT | 1 um | HPLC | 177 |
| Long_Splint4 | CTCTTGACACTGCACGAGGAACTATCGGAGGGTAACCCTAACCCTAACC | | | |
| | C/3InvdT/ | | | |
| AscI_NB19_ | GAGTCCACCATGAATTCGGCGCGCCATGTAACTGATTGCTGTACGCAAT | 1 um | HPLC | 178 |
| Long_Splint5 | CTCTTGACACTGCACGAGGAACTATCGGAGGGAACCCTAACCCTAACCC | | | |
| | T/3InvdT/ | | | |
| AscI_NB19_ | GAGTCCACCATGAATTCGGCGCGCCATGTAACTGATTGCTGTACGCAAT | 1 um | HPLC | 179 |
| Long_Splint6 | CTCTTGACACTGCACGAGGAACTATCGGAGGGACCCTAACCCTAACCCT | | | |
| | A/3InvdT/ | | | |

Annealing Biotin Adapters with Splints

The oligonucleotides were annealed together to make a duplex adapter set that has both the splint and adapter hybridized together. This was done in volume of 100 μLs in a PCR tube. An adapter mix that has 30 μM of phosphorylated UMI adapter and 5 μM of each splint permutation was made. The taq ligase buffer has high salt and was used for ensuring that the duplex DNA is stable at high temperatures once formed. The Taq ligation buffer was heated to 65 °C to dissolve the salts.

Using a thermocycler, the oligos were annealed, then first denatured at 95 °C and cooled by decreasing the temperature by 1 Degree®C/min until the temperature reached predicted Tm (85 °C). The adapter mix was then held at the Tm for 10 mins. After this, the adapter mix was cooled to 4 °C by cooling 1 °C/min. The annealed adapters were then held at 4 degrees.

### Restriction Digest of Biotin Tagged and Purified DNA Product

Since the streptavidin beads cannot bind to HMW DNA, it was needed to partially digest the DNA. The type of restriction enzyme used depends on how much of the subtelomere that would need to be captured. For bulk telomere sequencing the 6-base cutter BamHI was used that leaves ~4 kb of subtelomere sequence. EcoRI was used to release the adapters from streptavidin beads at a later step. If longer subtelomere sequences are needed (for chromosome specific assignment of telomeres), then an 8 base cutter can be used (e.g., Not1 or Asc1) with the biotin adapters that have a Not1 or Asc1 cut site instead. The tradeoff here is that fewer molecules are pulled down compared to the 6 bp cutter due to the properties of the streptavidin beads. 3 ul of EcoR1 HF (100,000 U/mL) was added to the purified tagged DNA in cutsmart, then digested for 15-30 min at 37 °C. For chromosome specific analysis, 3 μL of Asc1 was used.

Ligation of Biotin Telo Tag adapters to HMW DNA

A 1.100 dilution for the annealed adapters mix was made in 1× hifi TAQ buffer. The taq buffer was heated and then diluted 10 μl of buffer with 90 μL of water. The solution was cooled down by placing on ice. Then, 99 μL of this 1× hifi Buffer was taken and 1 μL of the annealed adapter mix was added. The components were then mixed in a protein lo-bind tube on ice and the tubes were placed in thermocycler and a cycling ligation program was run, wherein the program heats to 70 °C and denatures the DNA just enough to make sure the 3′ telomere overhang is not forming any secondary structures. The thermocycler then slowly cools to 45 °C where the ligation takes place. This was repeated for 15 cycles to allow for binding of the different permutations of the telomere adapter to capture all telomeres.

Cleanup of HMW DNA with Tagged Telomeres Using and Removing Excess Adapters Using SPRI Select Beads

It was needed to clean up the DNA to remove all the excess adapters that were in solution and were not ligated. The SPRI Select beads bottle was placed on the hula mixer rotating at 3 rpm for 1 hour. 1× cutsmart was made from 10× stock and warmed to 37 °C. The PCR tubes were then spun down from the previous step to collect all the liquid. The ligated reaction was pulled in a 1.5 mL protein lo bind tube. The ligated reaction was then dispensed to a new tube. The beads and DNA was mixed with wide orifice tips, mix 20 times, steadily, and the tube was placed on hula at rotating at 10 rpm for 7 mins.

The tube was then placed on magnet for 1 hr and 30 min. The supernatant very gently pipetted off and washed 2× with freshly made 85% ethanol. The Ethanol was taken off, spun down using a microcentrifuge, and placed back on magnet and any trace amounts of ethanol was removed using a P20 pipette. 200 μl (per 40 μg of input DNA) of the warmed 1× cutsmart was added, the tube was then removed from magnet and mix gently with rainin pipette tip (not wide orifice) for 20 times to break up the bead/DNA complex. The pooled samples were incubated at 37 °C for 15 mins and placed on magnet for 2 min. The amount of DNA was then measured using Qubit BR (1 μl of DNA).

### Biotin Pulldown of Tagged Telomeres Using Magnetic Streptavidin Beads

The MyOne Streptavidin C1 beads and binding solution were brought to room temp, and the bottle was placed on HuLa rotating at 3 rpm. 50 μl of MyOne Streptavidin C1 beads per 40 g of DNA input were taken and placed in 1.5 ml EP Eppendorf tube and placed on magnet for 2 min. 50 μl of supernatant was then taken off and afterwards the tube was taken off the magnet and resuspended in the original volume of beads used (300 μL of beads=300 μl of binding buffer). At this point the telomeres are being bound to the streptavidin and pipetting can shear the telomere and break off the adapter. The eppendorf tube was placed on a hula (taped or rubber banded horizontally with a 15-30 degree slight slant) and incubated at room temp for 20 mins at 1 rpm. The samples were placed on the magnet for 1 min or until the supernatant seems clear, then removed from magnet and added on top of beads 1 ml of DynaBeads high salt Washing Solution. The samples were placed on the hula or roller to continue resuspension and washing for 5 mins.

A digest solution was prepared to release the telomeres from the streptavidin beads, where the beads were placed back on magnet and 75 μl of digest solution was added to the beads. If multiple samples are being multiplexed for sequencing this is the step to pool them and do a pooled digestion: resuspend 1 sample and then move the bead/digest solution from tube 1 to the next tube to pool and digest the samples together. The samples were incubated for 2 h taped horizontally with a slight angle on hula inside the 37 °C incubator rotating at 2 rpm. The beads were then placed on magnet and the supernatant contains released telomeres.

### Nanopore Library Prep Using the LSK110 kit

The DNA from the digest step was measured using Qubit HSyour DNA, where a total DNA range of about 6-20 ng of DNA was measured in the lo-bind tube.

### Example 4—Bioinformatic Analysis of Telomere Length from Nanopore Profiling Data

Telomere length is maintained by telomerase around an equilibrium length distribution. To accurately measure telomere length, sequence analysis requires many telomere reads to capture the full distribution of lengths. For yeast, whole genome sequencing using nanopore profiling generated hundreds of reads per telomere. For human DNA, however, whole genome sequencing generated relatively few telomere reads.

Therefore, a telomere enrichment method was developed that generates thousands of telomere reads per sample (FIG. 3A). High molecular weight DNA was prepared from between 5-40 mg of DNA and ligated a splinted biotinylated oligonucleotide (TeloTag) to all telomeres. Cutting the DNA with restriction enzyme allowed efficient enrichment of telomere fragments using streptavidin beads. The optimal ratio of streptavidin beads to TeloTagged input genomic DNA was determined, which can capture ~15-20% of all input telomeres as determined by comparison to Southern blot (FIG. 5B). The TeloTag includes a barcode for multiplexing samples and a restriction site to release the bound fragments from the streptavidin beads.

A bioinformatic pipeline was developed to determine both “bulk” (all telomeres) and chromosomes end-specific telomere lengths. Guppy base-calling was used and filtered for reads containing 2 contiguous TTAGGG repeats as well as variant repeats and mapped the reads to a custom CHM13 reference genome using minimap2 and filtered for a mapq score of >40. Telomere length was calculated as the number of base pairs between the TeloTag and the subtelomere—telomere boundary (FIG. 3B). The human telomere subtelomere contains many “variant” telomere repeats such as TGAGGG, TCAGGG that differ from the canonical TTAGGG sequence due to mutation accumulation over time. The subtelomere boundary was set as the position where there is a significant deviation from the TTAGGG repeat base composition using a rolling window moving from the telomere into the subtelomere. This method differs from previous studies using 2×TTAGGG as the boundary as it incorporates the variant repeats into the telomere since they may contribute to the length regulation.

Known systematic errors with Guppy base-calling may affect the calculated telomere length, especially due to errors on the strand that contains CCCTAA repeats (C strand). To examine this, the length of the strand that contains TTAGGG (G strand) to the C strand was compared. The median difference in length was 344 bp (FIG. 5A). (The length differences were greater on longer telomere suggesting a systematic error in base calling). To determine whether the length determined by Guppy base calling represents the true length of the telomere repeats, the electrical signal was examined in the Fast5 files. An algorithm was established to count the repeated TTAGGG peaks in the signal data and found a good correlation of the number of repeats with telomere length determined by Guppy base-calling.

### Example 5—Telomere Enrichment Significantly Increases Telomeric Reads

To estimate the efficiency of enrichment of telomere reads per flow cell, genomic DNA that was not enriched on a minion flow cell was sequenced and a total of 2,234,309 reads were obtained that had 312 reads containing 3 consecutive TTAGGG repeats representing 0.001% telomere containing reads. After enrichment, generally obtain ~50-100,000 telomere reads from a total of ~200,000 reads. Thus, biotin enrichment allows for more than ~6000× telomere reads for each flow cell compared whole genome sequencing. Enrichment has the added advantage that there is significantly less other genomic DNA and thus available pores sequence primarily telomere reads. 5 samples were routinely multiplexed per flow cell and generated ~7000 telomere reads for each sample. This is not an upper limit, as most pores still remained unoccupied suggesting further multiplexing may be possible.

### Example 6—Nanopore Telomere Profiling Reports Telomere Length Similar to Southern Blot

DNA from whole blood from people of different ages was used and compared telomere length measured by Southern blot (FIG. 4) to nanopore sequencing (FIG. 4). Nanopore sequencing accurately represented the length differences between individuals determined on the Southern blot. Telomeres measured on a Southern blot include subtelomeric DNA on the terminal restriction fragment. Thus, the length of the distribution of telomere fragments on a Southern is slightly longer than the length of TTAGGG sequence determined by nanopore. The amount of this difference depends on what restriction enzyme was used to cut in the subtelomere.

For nanopore profiling cutting genomic DNA with EcoR1 generated shorter DNA fragments than cutting with PacI but there was no difference in calculated telomere length indicating fragment length did not have measurable effect on telomere length.

The nanopore length measurements were highly reproducible when multiple samples were run on same flow cell (inter-assay correlation of variation of 1.2%) (FIG. 6A) and between different flow cells (intra-assay variability of 0.48) (FIG. 6A). These compare favorably to the intra-assay variability of 2.2% for FlowFISH; and 7.9% qPCR and an inter-assay variability of 2.5% for FlowFISH and 25% for qPCR. To determine the reproducibility of the method between different laboratories, two different people preformed the assay on the same DNA; and samples were prepared and analyzed in two different laboratory settings (Johns Hopkins and UCSC). The inter-lab variation was X.X % (FIG. 6B) indicating the length data is highly correlated between labs, which is not the case for Southern blots.

### Example 7—Nanopore Sequencing Shows Population Distribution and Shortening with Age Similar to FlowFISH

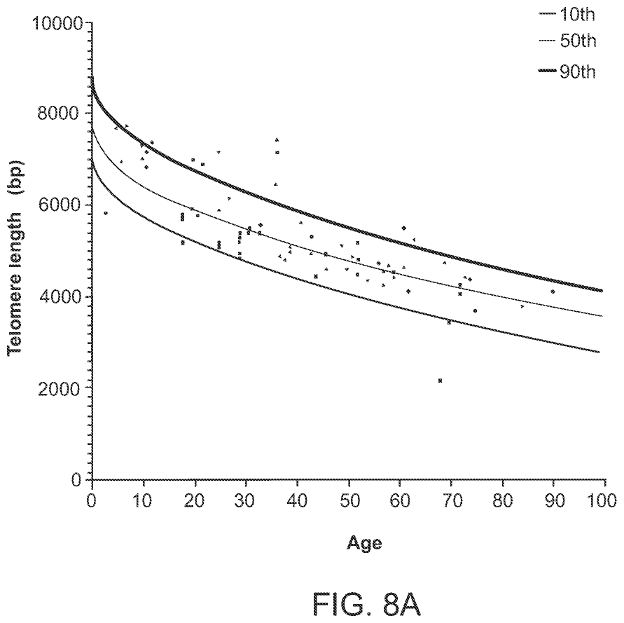

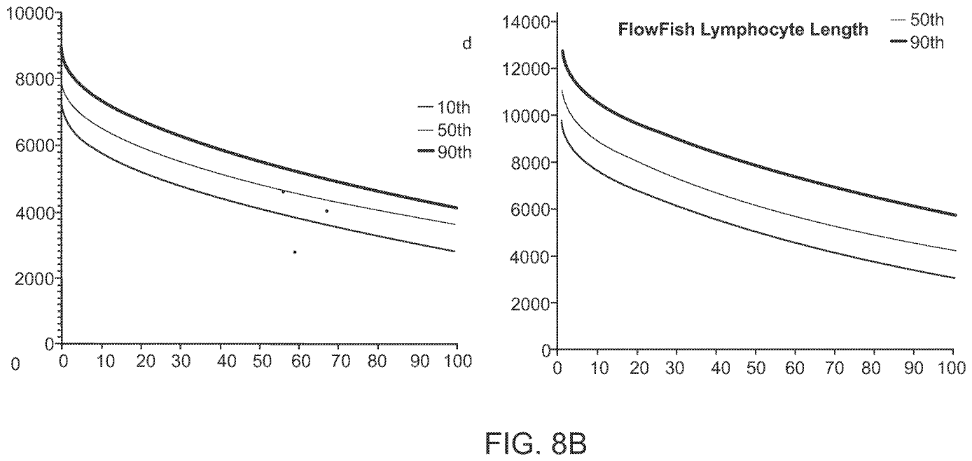

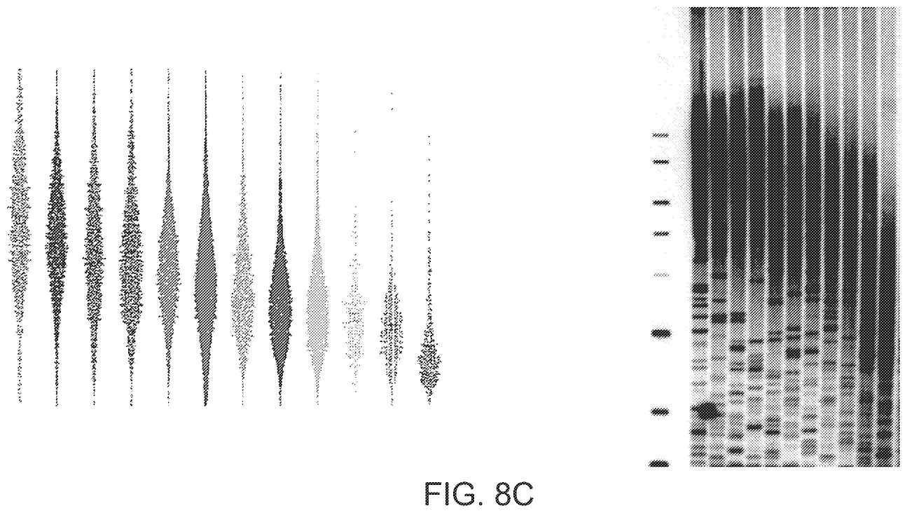

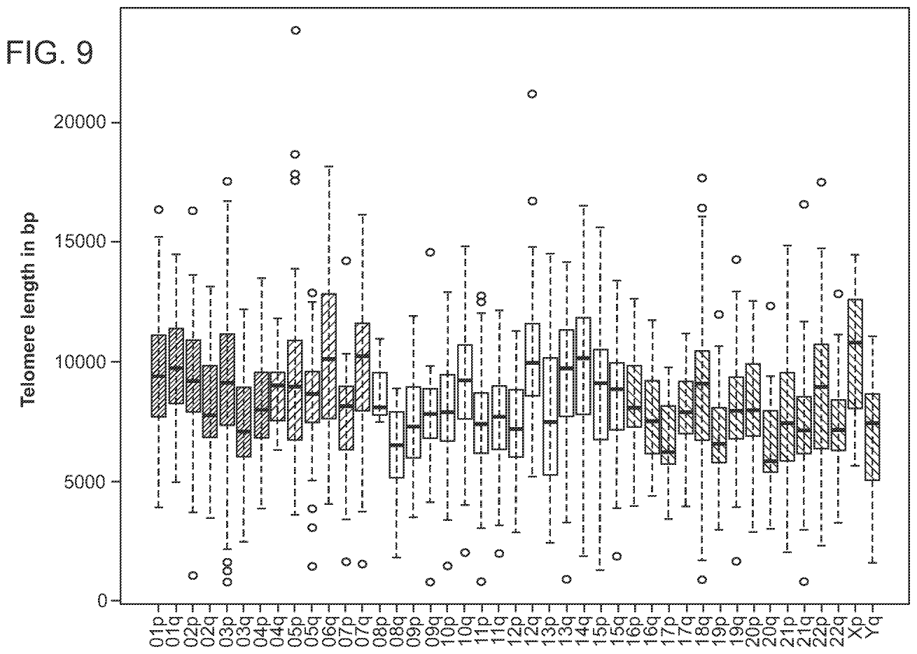

Telomere length shortens with age. FlowFISH, has established the normal range of telomere length in the population at a given age. Telomeres from whole blood and PBMCs were sequenced in ~150+ individuals ranging from 0 (newborn cord blood) to 91 years old. The range of median lengths was calculated across the population at each age to determine the 90^{th}, 50^{th} and 10^{th} percentile in the population as established previously for FlowFISH (FIG. 8A). Nanopore telomere profiling showed telomere length decreased with age as seen using Southern and FlowFISH. The rate of decrease with age (−50 bp/year) and the population distribution across ages were comparable to the previously published FlowFISH (−70 bp/year) (FIGS. 8A-8C). The y axis intercepts are different for the two data sets because the FlowFISH length in kilobases is inferred from a reference sample Southern blot. As noted above, Southern telomere fragments contain subtelomere sequences and so are longer than the region of TTAGGG repeats calculated by nanopore.

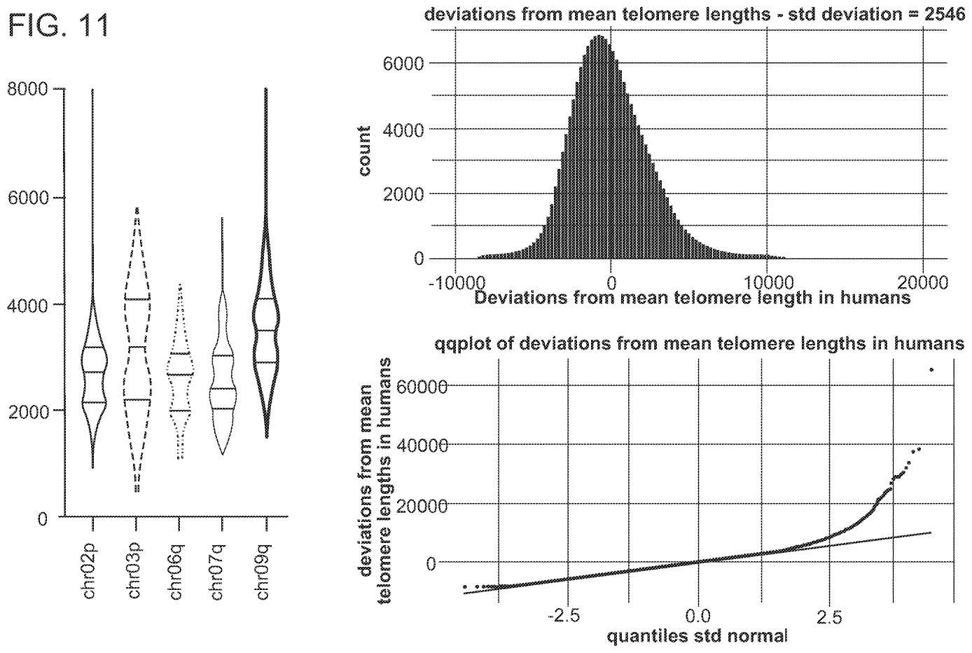

To determine the number of telomere reads required to obtain good approximation of the mean distribution length, ~130000 reads were collected from 44 individuals and measured the deviations in length from the mean of each individual (FIG. 8A). The resulting distribution was approximately gaussian (with some skew toward longer lengths) with a standard deviation of 2546. (FIG. 11). It was calculated that approximately 2500 reads are required to determine the bulk telomere mean within plus or minus 100 bp with 95% confidence. Using 10 mg of genomic DNA and multiplexing 15 samples per flow cells we reproducibly obtain more than 2500 tagged telomere reads.

Samples sequenced by nanopore were directly compared with those measured by Southern and FlowFISH. 12 samples with different telomere length. measured by nanopore (FIG. 8C) correlated with the distributions in the Southern Blot (FIG. 8C). 15 samples from individuals who previously had telomere lengths measured by FlowFISH were sequenced and it was found that the telomere length fell in similar percentile for their age using both assays. The slight differences in median telomere length between the two assays may be due to differences in cell types in the genomic DNA samples. FlowFISH can report on telomere lengths in specific cell types using cell surface markers while nanopore sequencing combined all lymphocytes. Thus, the differential signal in lymphocyte verses granulocytes that is used for telomere syndrome diagnosis would require further cell type specific sorting before nanopore sequencing.

### Example 8—Human Chromosome Ends have Unique Telomere Length Distributions that are Maintained with Aging

To determine whether human chromosome show end-specific telomere length as was found in yeast, telomeres from the HG002 cell line were initially examined for which a high quality reference genome is available. Human subtelomeres contain blocks of homology (parology blocks) shared between many different telomeres. To minimize mis-mapping, it was started with the best case of mapping HG002 reads to the HG002 genome. The telomere reads were mapped to individual chromosome ends and filtered for a mapq score of >40 and it was found that specific telomeres had significantly different length distributions. Analysis of the Mean showed statistically significant distributions of the end-specific distributions. No correlation was found of the length of telomeres with the length of the chromosome or between the two telomeres on a given chromosome.

To determine whether end-specific telomere lengths are conserved across individuals, a subset of telomeres were determined where sequence reads could be confidently mapped from diverse individuals to the terminal 30 kb of the reference genome. The acrocentric telomeres 13p 14p, 15p, 21p and 22p and the pseudoatutosomal Xp Yp were excluded as they frequently exchange between chromosomes. Chromosomes ends were identified that reproducibly mapped uniquely and to avoid age effect on telomere length, cord blood from 5 different individuals was examined.

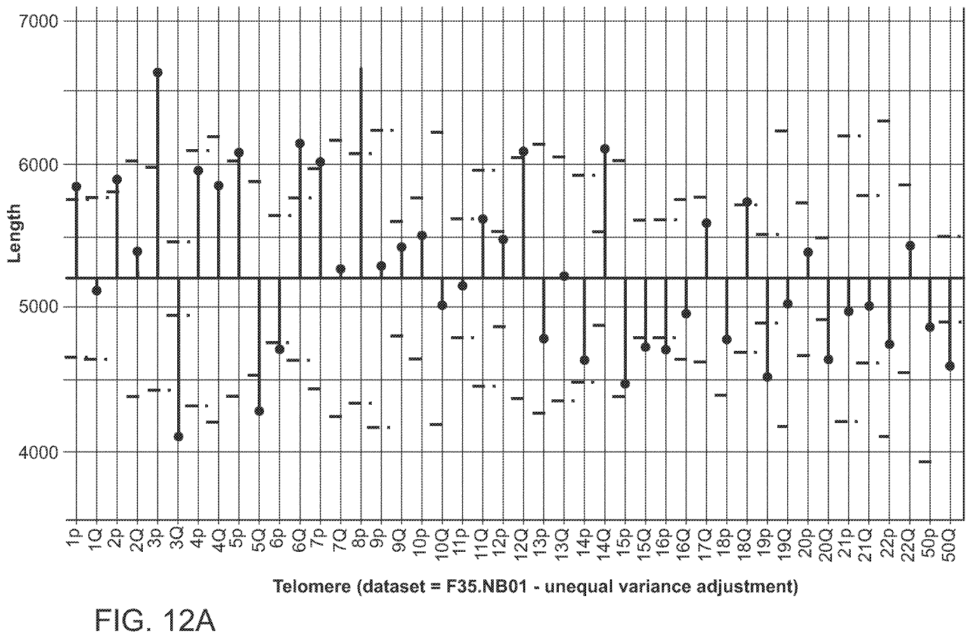

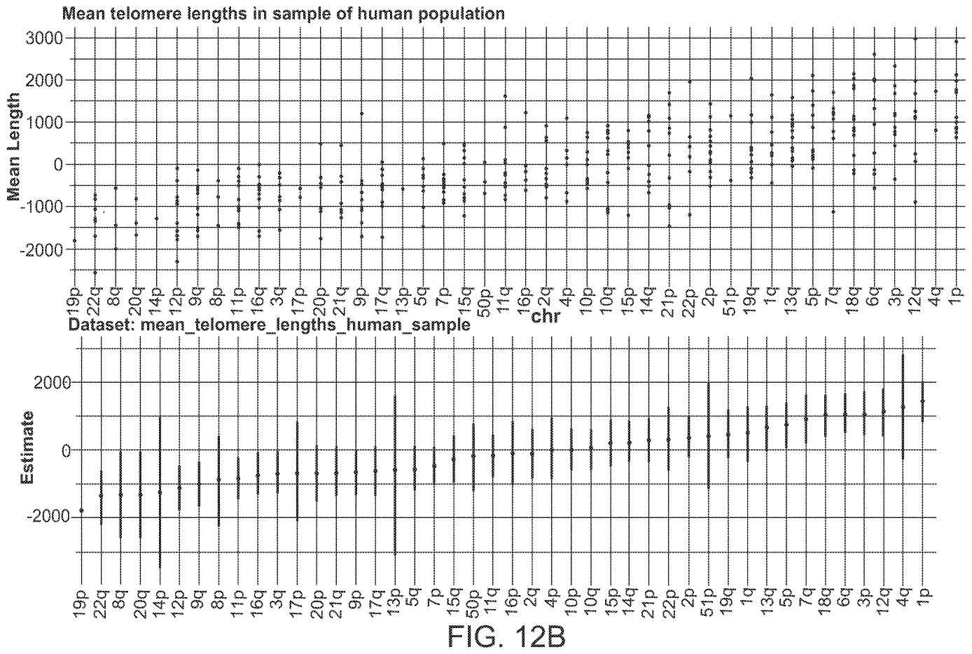

It was found that chromosome end specific telomere lengths are conserved between individuals (FIG. 12B).

To determine whether chromosome specific ends are maintained with age, 5 different individuals were compared across the age spectrum from 3 to 91 years. It was found that, despite the age differences, the same specific telomeres had a similar distribution suggesting telomere lengths on specific chromosomes are conserved across individuals.

The reads from 10 individuals with Short telomeres Syndrome were mapped to specific chromosome ends and found a similar set of a bimodal distribution of telomere lengths (FIGS. 12A-12B). Since this individual inherited one set of short telomeres from only one parent the bimodal distribution suggests that the maternal and paternal telomere length differences are maintained in the blood over many years.

### Example 9—Chromosome Specific Lengths are Conserved Across 70 Different Individuals

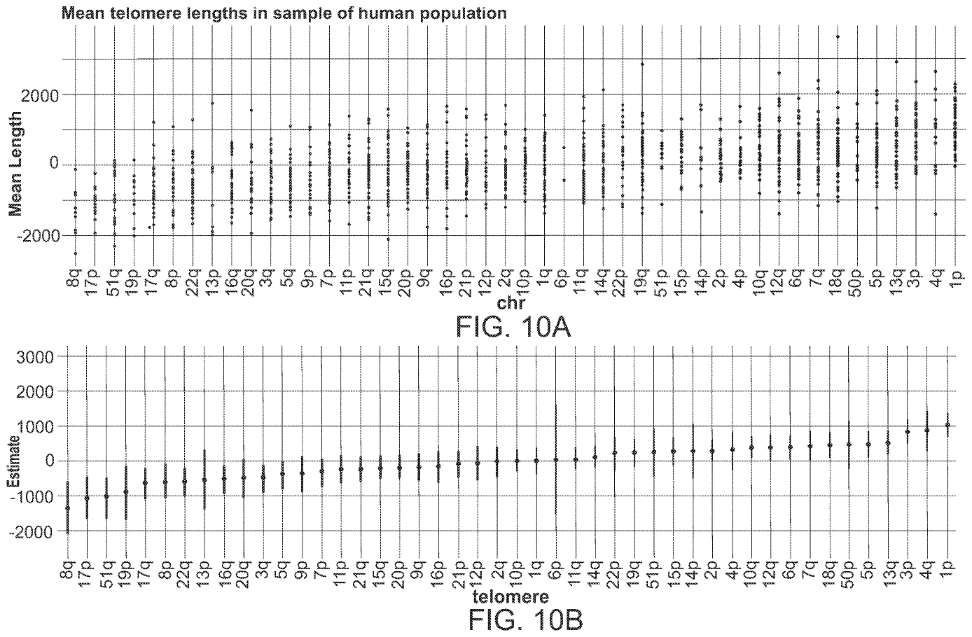

The analysis of individual telomere length was expanded across the age spectrum to include 70 samples where there was a minimum of 25 reads per telomere. To compare individuals of different ages, the mean length deviation of each telomere was calculated from the mean length of all telomeres for each individual (the grand mean). The resulting age corrected values are referred to as relative mean telomere lengths. FIGS. 10A-10B show the relative mean telomere lengths for each telomere for each individual and the average length deviation for each telomere over the whole population of 70 individuals together with simultaneous 95% confidence intervals. In the population examined, some telomeres were significantly longer on average than the grand mean, including 1p, 3q, 3p, 13q and 5 p. It is striking that for all individuals 1p telomere was reproducibly longer than the average telomere length. Other telomeres were significantly shorter including 8q, 17p, 51q, 19p and 17q. The set of telomeres that were found as specifically long or short with nanopore telomere profiling, is in concordance with studies that used qFISH to examine telomere length. These results suggest that the mechanisms that generate telomere-specific mean lengths may be conserved across the human population and that telomere shortening with age affects all telomeres to a similar extent, thus maintaining telomere-specific length differences established at an early age. This is consistent with previous experiments showing that individual telomeres in primary fibroblasts shorten at similar rates and yeast telomeres shorten at similar rates in the absence of telomerase.

To examine telomere length at birth, the 13 cord blood samples from newborns were studied and again found that specific telomeres were longer or shorter (FIG. 12B). The order of the longest telomeres was slightly different than the data in FIG. 10A, likely due to the smaller data set for cord blood, however of the top 10 longest telomeres in cord blood 9 were also in the top 10 in the 70 people sampled above. Similarly of the top 10 shortest telomeres in cord blood 6 were also in the top to in the older people. These data support the earlier conclusion that telomere length in leukocytes is established in the zygote and shortens with little fluctuation in the population with age.

What is claimed is:

1. A method for determining a telomere length of a DNA molecule from a biological sample, the method comprising:
(a) providing a telomere tagging (TeloTag) probe comprising an oligonucleotide with a biotin adapter;
(b) providing a splint oligonucleotide, wherein the splint oligonucleotide specifically binds to at least a portion of a telomere on the DNA molecule and to at least a portion of the telomere tagging probe;
(c) attaching the telomere tagging probe to the telomere to generate a tagged telomere sequence;
(d) contacting the tagged telomere sequence with a streptavidin-functionalized bead, wherein the biotin adapter of the tagged telomere sequence binds the streptavidin-functionalized bead;
(e) recovering the tagged telomere sequence; and
(f) analyzing the tagged telomere sequence, thereby determining the telomere length of the DNA molecule.

2. The method of claim 1, wherein the splint oligonucleotide is not blocked at its 3′ end.

3. The method of claim 1 or 2, wherein the attaching step (c) comprises ligating the telomere tagging TeloTag probe to the 3′ end of the telomere.

4. The method of claim 3, wherein the ligating comprises cycling ligation.

5. The method of claim 3 or 4, wherein the ligating comprises a Taq DNA ligase.

6. The method of any one of claims 1-5, wherein the recovering comprises using a magnet to separate the tagged telomere sequence bound to the streptavidin-functionalized bead.

7. The method of claim 6, wherein the recovering further comprises releasing the tagged telomere sequence from the streptavidin-functionalized bead using a restriction enzyme, wherein the restriction enzyme cleaves the tagged telomere sequence at a restriction enzyme-specific site.

8. The method of any one of claims 1-7, wherein the analyzing comprises sequencing the released tagged telomere sequence.

9. The method of claim 8, wherein the sequencing comprises nanopore sequencing.

10. A method for determining a telomere length of a DNA molecule from a biological sample, the method comprising:
(a) providing a telomere tagging probe, wherein the telomere tagging probe comprises a unique molecular identifier (UMI) sequence;
(b) providing a splint oligonucleotide, wherein the splint oligonucleotide specifically binds to at least a portion of a telomere on the DNA molecule and to at least a portion of the telomere tagging probe;
(c) attaching the telomere tagging probe to the telomere to generate a tagged telomere sequence;
(d) contacting the tagged telomere sequence with a forward primer and a reverse primer, wherein the forward primer binds to a subtelomere of the telomere, and the reverse primer binds to at least a portion of the telomere tagging probe;
(e) amplifying the tagged telomere sequence to generate an amplified tagged telomere sequence; and
(f) analyzing the amplified tagged telomere sequence, thereby determining the telomere length of the DNA molecule.

11. The method of claim 10, wherein the splint oligonucleotide is blocked at its 3′ end.

12. The method of claim 10, wherein the splint oligonucleotide is not blocked at its 3′ end.

13. The method of any one of claims 10-12, wherein the attaching step (c) comprises ligating the telomere tagging probe to the 3′ end of the telomere.

14. The method of claim 13, wherein the ligating comprises cycling ligation.

15. The method of claim 13 or 14, wherein the ligating comprises a Taq ligase.

16. The method of any one of claims 10-15, wherein the amplification comprises PCR amplification.

17. The method of claim 16, wherein the amplification comprises about 15 to about 35 rounds of PCR amplification.

18. The method of any one of claims 10-17, wherein the analyzing comprises sequencing the tagged telomere sequence.

19. The method of claim 18, wherein the sequencing comprises long read sequencing.

20. The method of any one of claims 1-19, wherein the telomere tagging probe further comprises a sample barcode sequence that is associated with the biological sample.

21. The method of any one of claims 1-20, wherein the biological sample comprises a blood sample.

22. The method of any one of claims 1-21, wherein the biological sample comprises a tissue sample.
